(19) United States
(12) Patent Application Publication
(10) Pub. No.: US 2022/0118081 A1
(43) Pub. Date: Apr. 21, 2022

(54) HIV vaccine regimens

(71) Applicant: Janssen Vaccines & Prevention B.V., Leiden (NL)

(72) Inventors: Frank WEGMANN, Leiden (NL); Maria Grazia PAU, Leiden (NL); Gijs HARDENBERG, Voorschoten (NL); Sietske Karla ROSENDAHL HUBER, Vleuten (NL); Sanne KROOS, Rotterdam (NL)

(21) Appl. No.: 17/451,408

(22) Filed: Oct. 19, 2021

(30) Foreign Application Priority Data
Oct. 20, 2020 (EP) 20202762.9
Nov. 12, 2020 (EP) 20207201.3

(51) Int. Cl. A61K 39/21; A61P 31/18

(52) U.S. Cl. CPC A61K 39/21; A61K 2039/5256; A61P 31/18

(57) ABSTRACT

Methods are described for generating an improved effective immune response against an HIV antigen in humans. The methods comprise administration of a first composition comprising an MVA vector followed by administration of second composition comprising a human adenovirus vector. The methods can be used for the treatment of HIV.

### FIELD OF THE INVENTION

This invention relates to the fields of medical microbiology, immunology and vaccines. In particular the invention relates to methods and compositions for obtaining a broad T-cell immune response to an HIV antigen in a subject which can be used to provide a treatment against HIV infection.

### BACKGROUND OF THE INVENTION

Infectious diseases are the second leading cause of death worldwide after cardiovascular disease but are the leading cause of death in infants and children. Vaccination is the most efficient tool for preventing a variety of infectious diseases. The goal of vaccination is to generate a pathogen specific immune response providing preferably long-lasting protection against infection. Despite the significant success of vaccines, development of safe and strong vaccines is still required due to the emergence of new pathogens, re-emergence of old pathogens and suboptimal protection.

Human Immunodeficiency Virus (HIV) infection, particularly HIV-1 infection, continues to be a significant cause of mortality and morbidity worldwide despite the advances in anti-retroviral therapy (ART) and implementation of various prevention strategies, mainly due to poor adherence and heterogeneous access. In 2017, approximately two million new HIV infections occurred and roughly one million people died of AIDS-related illness. Effective HIV vaccines are needed to control and ultimately end the AIDS pandemic.

Recombinant adenovirus vectors (rAd) are powerful inducers of cellular immune responses and have therefore come to serve as useful vectors for gene-based vaccines for viral and non-viral pathogens alike. Adenovirus-based vaccines have several advantages as human vaccines since they can be produced to high titers under GMP conditions and have proven to be safe and immunogenic in humans.

Modified Vaccinia Ankara (MVA) virus is related to Vaccinia virus, a member of the genera Orthopoxvirus in the family of Poxviridae. Poxviruses are known to be good inducers of CD8 T cell responses because of their intracytoplasmic expression. MVA has been engineered for use as a viral vector for recombinant gene expression or as recombinant vaccine.

Cell-mediated immunity (CMI) breadth describes the number of epitopic regions in a vaccine antigen recognized by the cellular immune response. In preclinical therapeutic HIV vaccine studies in non-human primates (NHPs), CMI breadth has been identified as an important correlate of efficacy (Borducchi et al., Nature 540 (2016) 284-287). Published preclinical mouse data suggests that repeated homologous immunization modifies T cell hierarchy with a narrowing and focus on immunodominant epitopes (Rollier et al., Vaccine 34 (2016) 4470-4474). Clinical studies assessed the influence of various vaccine components, immunization regimens and intervals on immunogenicity and CMI breadth in naïve trial participants and HIV infected individuals (e.g. Borthwick et al Mol Ther. 2014 February; 22(2): 464-475; Mothe et al EClinicalMedicine. 2019 Jun. 5; 11:65-80; Viegas et al PLoS One. 2018 Nov. 29; 13(11):e0206838), showing that these factors can have a profound impact. Various combinations of vectors, components, and regimens are possible. One of the many possible combinations for vaccine components that can be useful in HIV vaccine regimens are combinations of adenoviral vectors and poxviral vectors. Clinical trials wherein human adenovirus vectors encoding HIV antigens are used for initial immunization, followed by immunization with MVA vectors encoding HIV antigens, with 12 weeks between administration of the adenovirus vectors and the MVA vectors have been described (e.g. WO 2018/045267; Colby et al, 2020, Nature Medicine 26: 498-501; WO 2019/055888). WO 2018/229711 discloses MVA vectors encoding HIV Env antigens and use thereof also in heterologous administration regimens with human adenovirus vectors. The examples therein describe initial administration with adenovirus vectors followed by administration of the MVA vectors. Roshorm et al in the group led by Hanke (European journal of immunology 42: 3243-3255 (2012)) describe a vaccination regimen that includes an initial administration of ChAdV68.GagB (recombinant chimpanzee adeno virus 68 comprising the HIV antigen GagB) followed by administration of MVA.GagB. These authors also describe the reverse vaccination scheme but neither study nor describe CMI breadth, they highlight the attractiveness of chimpanzee adenoviral vectors compared to human adenoviral vectors for which they indicate major disadvantages, and they refer to then ongoing clinical trials with regimens that include chimpanzee adenovirus vectors and poxvirus vectors. The state of such and following trials and studies aiming at protective T-cell responses against HIV is reviewed by Hanke (T Hanke. 2019. Expert Review of Vaccines, 18:10, 1029-1041). This review indicates that in the initial studies that included arms with chimpanzee adenovirus and poxvirus (MVA), the frequency of effector T-cells was substantially higher for regimens where MVA followed chimpanzee adenovirus administration as compared to reverse regimens where chimpanzee adenovirus administration followed MVA administration, and in addition this review does not appear to refer to any later studies wherein poxvirus administration is followed by adenovirus administration, whereas it describes several further studies that include arms wherein adenovirus administration is followed by poxvirus administration.

The art describing heterologous vaccine regimens using adenovirus and poxvirus vectors is thus heavily biased towards initial administration of the human adenovirus vector followed by administration of a poxvirus vector.

There is still a need in the art for HIV vaccination strategies that improve immunogenicity and particularly CMI breadth. It is an object of the instant invention to provide vaccine compositions and regimens for their administration that improve the breadth and/or the magnitude of the induced immune response against HIV.

### SUMMARY OF THE INVENTION

In a first aspect, the invention provides for a vaccine combination for use in the treatment of HIV in a subject, comprising:

i) a first composition comprising a poxvirus vector comprising a polynucleotide that encodes an HIV antigen;
ii) a second composition comprising a human adenovirus vector comprising a polynucleotide that encodes an HIV antigen, wherein the first composition is for administration to the subject and the second composition is for administration to the subject less than six weeks after administration of the first composition.

In a second aspect the invention provides for a method for obtaining a broad T-cell immune response to an antigen in a subject, the method comprising:

i) the administration of a first composition comprising a poxvirus vector comprising a polynucleotide that encodes an HIV antigen;
ii) followed less than 6 weeks after administration of the first composition by the administration of a second composition comprising a human adenovirus vector comprising a polynucleotide that encodes an HIV antigen,
wherein the broad T-cell immune response is characterized by a significant increase of positive peptide pools from the HIV antigen in an interferon-gamma ELISpot assay as compared to the response obtained in the same assay when the first administration to the subject is with the second composition and the second administration to the subject is with the first composition with the same interval between the first and second administration.

In a third aspect, the invention provides for a kit comprising:
i) a first composition comprising a poxvirus vector comprising a polynucleotide that encodes an HIV antigen;
ii) a second composition comprising a human adenovirus vector comprising a polynucleotide that encodes an HIV antigen; and
iii) a manual of instructions detailing that the second composition is to be administered after administration of the first composition to a subject and that the time interval between the administration of the first and the second composition to the subject is less than 6 weeks, wherein the subject suffers from an HIV infection before the first composition is to be administered.

In another aspect, the invention provides:

a first composition comprising a poxvirus vector comprising a polynucleotide that encodes an HIV antigen, for use in combination with a second composition comprising a human adenovirus vector comprising a polynucleotide that encodes an HIV antigen; wherein the first composition is for administration to the subject and the second composition is for administration to the subject less than six weeks after administration of the first composition.

In another aspect, the invention provides:

a second composition comprising a human adenovirus vector comprising a polynucleotide that encodes an HIV antigen, for use in combination with a first composition comprising a poxvirus vector comprising a polynucleotide that encodes an HIV antigen; wherein the first composition is for administration to the subject and the second composition is for administration to the subject less than six weeks after administration of the first composition.

### BRIEF DESCRIPTION OF THE DRAWINGS

FIGS. 1A and 1B: Number of positive peptide pools determined by IFN-γ ELISpot. Splenocytes were isolated 2 weeks after the final dose were stimulated overnight with 17 PTE Env peptide pools in an IFN-γ ELISpot plate. A positive response was defined as >50 spot forming units (SFU)/10⁶ cells. Immunization regimens are depicted as follows: 2× Ad26,MVA: Dosing with Ad26.Mos4.HIV (2.5×10⁹ vector particles (vp)) at weeks 0 and 6 and MVA-BN-HIV (1×10⁷ TCID₅₀) at weeks 12 and 18. All other groups received one dose of Ad26.Mos4.HIV (5×10⁹ vp) and one dose of MVA-BN-HIV (2×10⁷ TCID₅₀), where the order of immunization is depicted by a comma and the interval between doses indicated by weeks (wks). Each dot represents a separate animal. Analysis-of variance was applied to square root transformed counts followed by a t-test. n.s. not significant * p<0.05, *** p<0.001, **** p<0.0001.

FIGS. 2A and 2B: Cellular breadth and magnitude depicted per pool. Splenocytes were isolated 2 weeks after the final dose were stimulated overnight with 17 PTE Env peptide pools in an IFN-γ ELISpot plate. A positive response was defined as >50 spot forming units (SFU)/10⁶ cells. Each peptide pool is presented with a different grayscale. Top numbers in each slice refer to the number of a peptide pool and bottom numbers are median SFU/10⁶ cells per pool. Total median responses and standard deviation (SD) are depicted below each pie chart. Immunization regimens are depicted as follows: 2× Ad26,MVA: Dosing with Ad26.Mos4.HIV (2.5×10⁹ vector particles (vp)) at weeks 0 and 6 and MVA-BN-HIV (1×10⁷ TCID₅₀) at weeks 12 and 18. All other groups received one dose of Ad26.Mos4.HIV (5×10⁹ vp) and one dose of MVA-BN-HIV (2×10⁷ TCID₅₀), where the order of immunization is depicted by a comma and the interval between doses indicated by weeks (wks).

### DETAILED DESCRIPTION OF THE INVENTION

It was surprisingly found herein that when a composition comprising a poxviral vector expressing a HIV antigen is administered before administration of a composition comprising a human adenoviral vector expressing the HIV antigen, a significantly higher CMI breadth and magnitude were found as compared to another tested vaccine regimen herein with a different order or number of immunizations. Greater cellular breadth has been indicated as favorable in a therapeutic vaccination. Advantageously, this effect was already observed with short vaccine regimens. Short vaccine regimens, i.e. regimens wherein the time interval between the first and the second administration is relatively short (such as a few weeks) have several advantages over long vaccine regimens, as the short timespan between doses may simplify the vaccine dosing regimen in a clinical setting. Such broad T-cell responses may be especially beneficial for HIV, which is known to exist in many variants and prone to mutations, so that a broader T-cell immune response towards many different epitopes may lower the chance that escape variants will arise.

Furthermore, the vaccine regimen as described herein requires fewer rounds of immunization to achieve a significantly higher CMI breadth and magnitude as compared to any other tested vaccine regimen herein which is advantageous as it reduces cost and increases uptake of the vaccine by the eligible population.

Accordingly, in a first aspect, the invention provides for a vaccine combination for use in the treatment of HIV in a subject comprising:

i) a first composition comprising a poxvirus vector comprising a polynucleotide that encodes a HIV antigen; and

ii) a second composition comprising a human adenovirus vector comprising a polynucleotide that encodes a HIV antigen,

wherein the first composition is for administration to the subject and the second composition is for administration to the subject less than six weeks after administration of the first composition.

The term “antigen” refers to an antigenic or immunogenic protein, polypeptide or peptide comprising one or more epitopes (of a pathogen) against which the vaccine combination aims to induce an immune response. For vaccination purposes, it is often beneficial if the same antigen, or the nucleic acid encoding that antigen, is administered several times. In one embodiment, the antigen encoding polynucleotide comprised in the poxvirus vector of the first composition is substantially identical, preferably identical, to the antigen encoding polynucleotide comprised in the adenoviral vector of the second composition. “Substantially identical” as used herein refers to the idea that the antigen might be slightly different, but should still elicit an immune response that would fully (or at least sufficiently) protect the vaccinated individual from the pathogen. For example, the antigen encoding polynucleotide in the second composition may be slightly different, e.g. may have at least 95, 96, 97, 98 or 99% sequence identity with the antigen encoding polynucleotide of the first composition. Preferably the antigen that is encoded by the polynucleotide comprised in the poxvirus vector has at least one epitope in common with the antigen that is encoded by the polynucleotide comprised in the human adenovirus vector. More preferably, a contiguous stretch of at least 10, 20, 30, 40, 50, 60, 70, 80, 90 or 100 of the amino acids in the sequence of the antigen that is encoded by the polynucleotide comprised in the poxvirus vector is identical to the amino acid sequence of the antigen that is encoded by the polynucleotide comprised in the human adenovirus vector. Most preferably, the amino acid sequence of the antigen that is encoded by the polynucleotide comprised in the poxvirus vector is identical to the amino acid sequence of the antigen that is encoded by the polynucleotide comprised in the human adenovirus vector.

Preferably the pox viral vector according to the invention is an orthopox viral vector; preferably the pox viral vector used is the recombinant vaccinia virus Modified Vaccinia virus Ankara (MVA) vector; MVA is a highly attenuated pox viral vector. Preferably the pox viral vector is non-replicating or replication impaired. In additional preferred embodiments, the MVA virus vector is MVA-BN or derivatives thereof.

Chorioallantois vaccinia virus Ankara virus (CVA) was maintained in the Vaccination Institute, Ankara, Turkey for many years and used as the basis for vaccination of humans. The attenuated CVA-virus MVA (Modified Vaccinia Virus Ankara) was obtained by serial propagation (more than 570 passages) of the CVA on primary chicken embryo fibroblasts (CEF).

However, due to the often severe post-vaccination complications associated with vaccinia viruses, there were several attempts to generate a more attenuated, safer vaccine. As a result of the passaging used to attenuate MVA, there are a number of different strains or isolates, depending on the number of passages conducted in CEF cells. Strains of MVA having enhanced safety profiles for the development of safer products, such as vaccines or pharmaceuticals, have been developed, for example by Bavarian Nordic. MVA was further passaged by Bavarian Nordic and is designated MVA-BN. A representative sample of MVA-BN was deposited on Aug. 30, 2000 at the European Collection of Cell Cultures (ECACC) under Accession No. V00083008. MVA-BN is further described in WO 02/42480 (see also e.g., U.S. Pat. Nos. 6,761,893 and 6,913,752 and US 2003/0206926) and WO 03/048184 (US 2006/0159699. MVA as well as MVA-BN lacks approximately 15% (31 kb from six regions) of the genome compared with ancestral CVA virus. The deletions affect a number of virulence and host range genes, as well as the gene for Type A inclusion bodies.

In various embodiments, the MVA or MVA used for generating the recombinants suitable for the present invention are MVA-572, MVA-575, MVA-1721, MVA as deposited as ATCC® VR-1508™, MVA as deposited as ATCC® VR-1566™, ACAM3000 MVA, MVA-BN or any similarly attenuated MVA strain. In preferred embodiments, the MVA used for generating the recombinants are MVA-575, MVA as deposited as ATCC® VR-1508™, MVA as deposited as ATCC® VR-1566™, ACAM3000 MVA and MVA-BN. Preferably the MVA used for generating the recombinants is MVA-BN.

MVA-572 was deposited at the European Collection of Animal Cell Cultures (ECACC, Vaccine Research and Production Laboratory, Public Health Laboratory Service, Centre for Applied Microbiology and Research, Porton Down, Salisbury, Wiltshire SP4 0JG, United Kingdom) with the deposition number ECACC V94012707 on Jan. 27, 1994. MVA-575 was deposited under ECACC V00120707 on Dec. 7, 2000. Acam3000 MVA was deposited at the American Type Culture Collection (ATCC) under Accession No.: PTA-5095 on Mar. 27, 2003 (American Type Culture Collection, 10801 University Blvd., Manassas, Va. 20110-2209, USA). MVA-1721 was deposited as CNCM 1721 at the Collection Nationale de Cultures de Microorganisms, Institute Pasteur. MVA-BN was deposited on Aug. 30, 2000 at the ECACC under number V00083008. MVA-BN has been described in WO 02/042480.

Also encompassed by the invention are derivatives or variants of any of the MVA viruses or MVA-BN described herein. “Derivatives” or “variants” of MVA or MVA-BN refer to MVA or MVA-BN viruses exhibiting essentially the same replication characteristics as the MVA or MVA-BN to which it refers, but exhibiting differences in one or more parts of their genomes. Viruses having the same “replication characteristics” as the deposited virus are viruses that replicate with similar amplification ratios as the deposited strain in chicken embryo fibroblasts (CEF) cells and the cell lines HaCat (Boukamp et al. (1988), J Cell Biot 106: 761-771), the human bone osteosarcoma cell line 143B (ECACC No. 91112502), the human embryo kidney cell line 293 (ECACC No. 85120602), and the human cervix adenocarcinoma cell line HeLa (ATCC No. CCL-2). Tests and assay to determine these properties of MVA, its derivatives and variants are well known to the skilled person, such as the cell line permissivity assay as described in WO 02/42480. In an exemplary cell line permissivity assay, mammalian cell lines are infected with the parental and derivative or variant MVA virus at a low multiplicity of infection per cell, i.e., 0.05 infectious units per cell (5×10⁴ TCID₅₀). Following absorption of 1 hour the virus inoculum is removed and the cells washed three times to remove any remaining unabsorbed viruses. Fresh medium supplemented with 3% FCS is added and infections are left for a total of 4 days (at 37° C., 5% CO₂) where viral extracts can be prepared. The infections are stopped by freezing the plates at −80° C. for three times. Virus multiplication and cytopathic effects (CPE) are subsequently determined on CEF cells using methods well known to the skilled person such as those described in Carroll and Moss (1997), Virology 238, 198-211.

More specifically, MVA-BN or a derivative or variant of MVA-BN preferably has the capability of reproductive replication in CEF cells, but no capability of reproductive replication in the human keratinocyte cell line HaCat (Boukamp et al (1988), J. Cell Biol. 106:761-771), the human bone osteosarcoma cell line 143B (ECACC Deposit No. 91112502), the human embryo kidney cell line 293 (ECACC Deposit No. 85120602), and the human cervix adenocarcinoma cell line HeLa (ATCC Deposit No. CCL-2). Additionally, a derivative or variant of MVA-BN has a virus amplification ratio at least two-fold less, preferably three-fold less than MVA-575 in Hela cells and HaCaT cell lines. Tests and assays for these properties of MVA variants are described in WO 02/42480 or in the exemplary cell line permissivity assay as described above.

The term “not capable of reproductive replication” or “no capability of reproductive replication” is, for example, described in WO 02/42480, which also teaches how to obtain MVA having the desired properties as mentioned above. The term applies to a virus that has a virus amplification ratio at 4 days after infection of less than 1 using the assays described in WO 02/42480 or in U.S. Pat. No. 6,761,893.

The term “fails to reproductively replicate” refers to a virus that has a virus amplification ratio at 4 days after infection of less than 1. Assays described in WO 02/42480 or in U.S. Pat. No. 6,761,893 are applicable for the determination of the virus amplification ratio.

Methods to obtain recombinant poxviruses or to insert exogenous coding sequences into a poxviral genome are well known to the person skilled in the art. For example, methods for standard molecular biology techniques such as cloning of DNA, DNA and RNA isolation. Western blot analysis, RT-PCR and PCR amplification techniques, techniques for the handling and manipulation of viruses, and techniques and know-how for the handling, manipulation and genetic engineering of MVA, are described in widely available textbooks and laboratory manuals.

For the generation of the various recombinant MVAs disclosed herein, different methods can be applicable. The DNA sequence to be inserted into the virus can be placed into an E. coli plasmid construct into which DNA homologous to a section of DNA of the MVA has been inserted. Separately, the DNA sequence to be inserted can be ligated to a promoter. The promoter-gene linkage can be positioned in the plasmid construct so that the promoter-gene linkage is flanked on both ends by DNA homologous to a DNA sequence flanking a region of MVA DNA containing a non-essential locus. The resulting plasmid construct can be amplified by propagation within E. coli bacteria and isolated. The isolated plasmid containing the DNA gene sequence to be inserted can be transfected into a cell culture, e.g., of chicken embryo fibroblasts (CEFs), at the same time the culture is infected with MVA. Recombination between homologous MVA DNA in the plasmid and the viral genome, respectively, can generate an MVA modified by the presence of foreign DNA sequences.

According to a preferred embodiment, a cell of a suitable cell culture as, e.g., CEF cells, can be infected with a poxvirus. The infected cell can be, subsequently, transfected with a first plasmid vector comprising a foreign or heterologous gene or genes, preferably under the transcriptional control of a poxvirus expression control element. As explained above, the plasmid vector also comprises sequences capable of directing the insertion of the exogenous sequence into a selected part of the poxviral genome. Optionally, the plasmid vector also contains a cassette comprising a marker and/or selection gene operably linked to a poxviral promoter.

Non-limiting examples of suitable promoters for the poxvirus vectors include the 30K promoter, the 13 promoter, the PrS promoter, the PrS5E promoter, the Pr7.5K, the Pr13.5 long promoter, the PrHyb promoter, the 40K promoter, the MVA-40K promoter, the FPV 40K promoter, 30 k promoter, the PrSynllm promoter, and the PrLE1 promoter. Additional promoters are further described in WO 2010/060632, WO 2010/102822, WO 2013/189611 and WO 2014/063832. In certain embodiments, the HIV antigen is Env and the promoter used to regulate the expression of the antigen in the poxvirus vector is PrHyb; in certain embodiments, the HIV antigen is GagPol and the promoter used to regulate the expression of the antigen in the poxvirus vector is Fri 3.5 long (see e.g. WO 2018/229711 for examples of such vectors).

Suitable marker or selection genes are, e.g., the genes encoding the green fluorescent protein, β-galactosidase, neomycin-phosphoribosyltransferase or other markers. The use of selection or marker cassettes simplifies the identification and isolation of the generated recombinant poxvirus. However, a recombinant poxvirus can also be identified by PCR technology. Subsequently, a further cell can be infected with the recombinant poxvirus obtained as described above and transfected with a second vector comprising a second foreign or heterologous gene or genes. In case, this gene shall be introduced into a different insertion site of the poxviral genome, the second vector also differs in the poxvirus-homologous sequences directing the integration of the second foreign gene or genes into the genome of the poxvirus. After homologous recombination has occurred, the recombinant virus comprising two or more foreign or heterologous genes can be isolated. For introducing additional foreign genes into the recombinant virus, the steps of infection and transfection can be repeated by using the recombinant virus isolated in previous steps for infection and by using a further vector comprising a further foreign gene or genes for transfection. Alternatively, the steps of infection and transfection as described above are interchangeable, i.e., a suitable cell can at first be transfected by the plasmid vector comprising the foreign gene and, then, infected with the poxvirus. As a further alternative, it is also possible to introduce each foreign gene into different viruses, co-infect a cell with all the obtained recombinant viruses and screen for a recombinant including all foreign genes. A third alternative is ligation of DNA genome and foreign sequences in vitro and reconstitution of the recombined vaccinia virus DNA genome using a helper virus. A fourth alternative is homologous recombination in E. coli or another bacterial species between a vaccinia virus genome, such as MVA, cloned as a bacterial artificial chromosome (BAC) and a linear foreign sequence flanked with DNA sequences homologous to sequences flanking the desired site of integration in the vaccinia virus genome.

One or more nucleic acid sequences encoding at least one antigen that can be used according to embodiments of the invention can be inserted into any suitable part of the poxvirus or poxviral vector. In a preferred aspect, the poxvirus used for the present invention includes an MVA virus or viral vector, preferably an MVA-BN virus or viral vector. Suitable parts of the MVA virus into which one or more nucleic acids of the present disclosure can be inserted include non-essential parts of the MVA virus.

An adenovirus that can be used according to the invention is a human adenovirus (HAdV, or AdHu). In the invention, a human adenovirus is meant if referred to as Ad without indication of species, e.g. the brief notation “Ad26” means the same as HAdV26, which is human adenovirus serotype 26. Also as used herein, the notation “rAd” means recombinant adenovirus, e.g., “rAd26” refers to recombinant human adenovirus 26.

Most advanced studies have been performed using human adenoviruses, and human adenoviruses are used according to the invention. A recombinant adenovirus according to the invention is thus based upon a human adenovirus. In preferred embodiments, the recombinant adenovirus is based upon a human adenovirus serotype 5, 11, 26, 34, 35, 48, 49, 50, 52, etc. According to a particularly preferred embodiment of the invention, an adenovirus is a human adenovirus of serotype 26. Advantages of these serotypes include a low seroprevalence and/or low pre-existing neutralizing antibody titers in the human population, and experience with use in human subjects in clinical trials.

Preferably, the adenovirus vector is a replication deficient recombinant viral vector, such as rAd26, rAd35, rAd48, rAd5HVR48, etc.

In a preferred embodiment of the invention, the adenoviral vectors comprise capsid proteins from rare serotypes, e.g. including Ad26. In the typical embodiment, the vector is an rAd26 virus. An “adenovirus capsid protein” refers to a protein on the capsid of an adenovirus (e.g., Ad26, Ad35, rAd48, rAd5HVR48 vectors) that is involved in determining the serotype and/or tropism of a particular adenovirus. Adenoviral capsid proteins typically include the fiber, penton and/or hexon proteins. As used herein a “capsid protein” for a particular adenovirus, such as an “Ad26 capsid protein” can be, for example, a chimeric capsid protein that includes at least a part of an Ad26 capsid protein. In certain embodiments, the capsid protein is an entire capsid protein of Ad26. In certain embodiments, the hexon, penton and fiber are of Ad26.

One of ordinary skill in the art will recognize that elements derived from multiple serotypes can be combined in a single recombinant adenovirus vector. Thus, a chimeric adenovirus that combines desirable properties from different serotypes can be produced. Thus, in some embodiments, a chimeric adenovirus of the invention could combine the absence of pre-existing immunity of a first serotype with characteristics such as temperature stability, assembly, anchoring, production yield, redirected or improved infection, stability of the DNA in the target cell, and the like. See for example WO 2006/040330 for chimeric adenovirus Ad5HVR48, that includes an Ad5 backbone having partial capsids from Ad48, and also e.g. WO 2019/086461 for chimeric adenoviruses Ad26HVRPtr1, Ad26HVRPtr12, and Ad26HVRPtr13, that include an Ad26 virus backbone having partial capsid proteins of Ptr1, Ptr12, and Ptr13, respectively).

In certain embodiments the recombinant adenovirus vector useful in the invention is derived mainly or entirely from Ad26 (La, the vector is rAd26). In some embodiments, the adenovirus is replication deficient, e.g., because it contains a deletion in the E1 region of the genome. For adenoviruses being derived from non-group C adenovirus, such as Ad26 or Ad35, it is typical to exchange the E4-orf6 coding sequence of the adenovirus with the E4-orf6 of an adenovirus of human subgroup C such as Ad5. This allows propagation of such adenoviruses in well-known complementing cell lines that express the E1 genes of Ad5, such as for example 293 cells, PER.C6 cells, and the like (see, e.g. Havenga, et al., 2006, J Gen Virol 87: 2135-43; WO 03/104467). However, such adenoviruses will not be capable of replicating in non-complementing cells that do not express the E1 genes of Ad5.

The preparation of recombinant adenoviral vectors is well known in the art. For example, preparation of rAd26 vectors is described, in WO 2007/104792 and in Abbink et al., 2007 Virology 81: 4654-63. Exemplary genome sequences of Ad26 are found in GenBank Accession EF 153474 and in SEQ ID NO:1 of WO 2007/104792.

Typically, an adenovirus vector useful in the invention is produced using a nucleic acid comprising the entire recombinant adenoviral genome (e.g., a plasmid, cosmid, or baculovirus vector).

The adenovirus vectors useful in the invention are typically replication deficient. In these embodiments, the virus is rendered replication deficient by deletion or inactivation of regions critical to replication of the virus, such as the E1 region. The regions can be substantially deleted or inactivated by, for example, inserting a gene of interest, such as a gene encoding an antigenic HIV protein, e.g. an HIV Env protein (usually linked to a promoter), within the region. In some embodiments, the vectors of the invention can contain deletions in other regions, such as the E2, E3 or E4 regions, or insertions of heterologous genes linked to a promoter within one or more of these regions. For E2- and/or E4-mutated adenoviruses, generally E2- and/or E4-complementing cell lines are used to generate recombinant adenoviruses. Mutations in the E3 region of the adenovirus need not be complemented by the cell line, since E3 is not required for replication.

Non-limiting examples of suitable promoters for the adenovirus vectors include the immediate early promoter of CMV (CMV promoter) and the Rous Sarcoma Virus Long Terminal Repeat promoter (RSV promoter). Preferably, the promoter is located upstream of the heterologous gene of interest within an expression cassette. A non-limiting example of a CMV promoter sequence that can be used in an adenovirus vector to drive expression of the HIV antigen is provided in SEQ ID NO: 24 of WO 2017/102929. Preferably, the promoter used in the human adenovirus vector of the invention is the CMV promoter.

A packaging cell line is typically used to produce sufficient amount of adenovirus vectors of the invention. A packaging cell is a cell that comprises those genes that have been deleted or inactivated in a replication-defective vector, thus allowing the virus to replicate in the cell. Suitable packaging cell lines include, for example, PER.C6, 911, 293, CAP, and E1 A549.

In certain embodiments, a recombinant adenovirus according to the invention is deficient in at least one essential gene function of the E1 region, e.g. the E1a region and/or the E1 b region, of the adenoviral genome that is required for viral replication. In certain embodiments, an adenoviral vector according to the invention is deficient in at least part of the non-essential E3 region. In certain embodiments, the vector is deficient in at least one essential gene function of the E1 region and at least part of the non-essential E3 region. The adenoviral vector can be “multiply deficient,” meaning that the adenoviral vector is deficient in one or more essential gene functions in each of two or more regions of the adenoviral genome. For example, the aforementioned E1-deficient or E1-, E3-deficient adenoviral vectors can be further deficient in at least one essential gene of the E4 region and/or at least one essential gene of the E2 region (e.g., the E2A region and/or E2B region).

In a preferred embodiment of the invention, the secondly administered vector is an adenovirus vector, and preferably a rAd26 vector, most preferably a rAd26 vector with at least a deletion in the E1 region of the adenoviral genome, e.g. such as that described in Abbink, J Virol, 2007. 81(9): p. 4654-63. Typically, the nucleic acid sequence encoding at least one antigen according to embodiments of the invention (e.g. an HIV envelope protein and/or other antigens) is cloned into the E1 and/or the E3 region of the adenoviral genome.

In certain embodiments, the HIV antigen is selected from HIV-1 Group Antigen (Gag), Polymerase (Pol), and/or Envelope (Env) proteins, or an antigenic part thereof. In preferred embodiments, the HIV antigen encoded in the poxvirus vector and in the adenovirus vector is an envelope (Env) protein or an immunogenic part thereof.

In one embodiment, the antigen is a “mosaic” antigen derived from HIV-1 Gag, Poi and/or Env antigens. Such mosaic antigens have been described before and developed in an attempt to provide maximal coverage of potential T-cell epitopes (e.g., Barouch et al, Nat Med 2010, 16: 319-323). The mosaic antigens are similar in length and domain structure to wild-type, naturally occurring HIV-1 antigens. For example, mosaic HIV antigens described and used in vaccines include those described in Barouch et al, supra, and for instance in WO 2010/059732, or in WO 2017/102929. Non-limiting examples of suitable mosaic HIV antigens (e.g. Table 1) that can be used in the instant invention include one or more of: (i) mosaic Gag antigen sequences as set forth in SEQ ID NO: 1 (“mos1.Gag”) or SEQ ID NO: 2 (“mos2.Gag”); (ii) mosaic Poi antigen sequences as set forth in SEQ ID NO: 3 (“mos1.Pol”) or SEQ ID NO: 4 (“mos2.Pol”); (iii) mosaic Env antigen as set forth in SEQ ID NO: 5 (“mos1.Env”) or SEQ ID NO: 6 (“mos2S.Env”); or fusions thereof, such as mos1.GagPol (SEQ ID NO: 11) or mos2.GagPol (SEQ ID NO: 12). Examples of suitable nucleic acid sequences encoding these antigens are set forth in SEQ ID NO: 7 (encoding mos1.GagPol), SEQ ID NO: 8 (encoding mos2.GagPol), SEQ ID NO: 9 (encoding mos1.Env), and SEQ ID NO: 10 (encoding mos2S.Env).

In a preferred embodiment of the invention, the polynucleotide encodes an Env polypeptide, e.g. in certain embodiments the polynucleotide may encode mos1.Env or mos2S.Env. In certain embodiments, a combination of Ad26 vectors is used, wherein each Ad26 vector comprises a polynucleotide encoding an antigen as indicated above, and the combination of Ad26 vectors together encodes a combination of the antigens as indicated above, for instance a combination of (i) mos1.GagPol, (ii) mos2.GagPol, (iii) mos1.Env, and (iv) mos2S.Env. Such combinations of vectors can be mixed in a single composition (see e.g. WO 2017/102929).

Other examples of HIV Gag, Pol, Env antigen sequences, or other HIV antigen sequences such as Nef, Tat, Rev, Vif, Vpr, or Vpu, are available to the skilled person from public databases, such as GenBank. Many different variants of HIV antigens have been described and could be used in the invention. Another non-limiting example would be the HIV T-cell immunogens described in WO 2013/110818.

In certain embodiments, further components can be administered to a subject to which the vectors are administered according to the invention, e.g. by additionally administering isolated HIV Env protein antigen, such as gp140 protein (see e.g. WO 2017/102929, for instance one or both proteins having amino acids 30-708 of SEQ ID NO: 7 of WO 2017/102929 and/or amino acids 30-724 of SEQ ID NO: 36 of WO 2017/102929).

As shown herein, a vaccine regimen comprising administration of a first composition comprising a poxviral vector followed by a second composition comprising an adenoviral vector significantly increases CMI breadth and magnitude in subjects receiving that vaccine regimen, as compared to the reverse regimen wherein adenoviral vector administration is followed by poxvirus vector administration. Such an immunization regimen inducing high CMI breadth and magnitude have been shown to be particularly efficacious as therapeutic HIV vaccine.

Accordingly, in an embodiment, the invention provides for a vaccine combination as described herein and a method as described herein for inducing a therapeutic immune response in an HIV infected subject.

In certain embodiments, the vaccine combination for use in the treatment of AIDS or an infection with HIV is given concurrently or in addition to treatment with anti-retroviral therapy (ART).

As used herein, “treatment of HIV in a subject” means administration of the vaccine to induce a therapeutic immune response against HIV or against cells that express (epitopes) of HIV in a subject which leads to at least reduction of the level of and preferably complete removal of HIV infection, which results in at least slowing and preferably stopping the progress of a disease caused by HIV such as AIDS and/or symptoms thereof. HIV/AIDS is a spectrum of conditions caused by infection with the human immunodeficiency virus (HIV). HIV is spread primarily by unprotected sex (including anal and oral sex), contaminated blood transfusions, hypodermic needles, and from mother to child during pregnancy, delivery or breastfeeding.

As used herein, the term “therapeutic immunity” or “therapeutic immune response” means that the HIV infected vaccinated subject is able to control an infection with the pathogenic agent, i.e., HIV, against which the vaccination was directed. Typically, the administration of the vaccine combination for use in the treatment of HIV in a subject as described herein will have a therapeutic aim to generate an immune response against HIV after HIV infection or development of symptoms characteristic of HIV infection. Preferably, the methods of the invention are for therapeutic purposes, such as for therapeutic vaccination, in which the compositions and vaccines described herein are administered to a subject already infected with HIV. Thus, the eligible population for treatment of HIV in a subject is preferably HIV-infected subjects, and preferably HIV-infected human subjects. The terms “HIV infection” and “HIV-infected” as used herein refer to invasion of a human host by HIV. As used herein, “an HIV-infected subject” refers to a subject in whom HIV has invaded and subsequently replicated and propagated within the host, thus causing the host to be infected with HIV or have an HIV infection or symptoms thereof.

In a preferred embodiment of the invention, the immune response is or comprises a cellular immune response, preferably a CD8+ T-cell response.

In one embodiment, the polynucleotide that encodes an HIV antigen comprised in the poxvirus vector of the first composition is substantially identical, preferably identical, to polynucleotide that encodes an HIV antigen comprised in the human adenoviral vector of the second composition as described herein.

In certain embodiments, the time interval between administration of the first and the second composition is less than 6 weeks such as for example 4 to 25 days, preferably 10 to 18 days. In certain the embodiments the time interval is about two weeks (14 days).

The first composition comprising the poxvirus vector of the invention and the second composition comprising the adenovirus vector of the invention are preferably pharmaceutical compositions that may comprise any pharmaceutically acceptable excipient including at least one of a carrier, filler, preservative, solubilizer and diluent. In the present context, the term “Pharmaceutically acceptable” means that the carrier or excipient, at the dosages and concentrations employed, will not cause any unwanted or harmful effects in the subjects to which they are administered. Such pharmaceutically acceptable excipients and carriers are well known in the art and for instance described in textbooks and manuals. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The precise nature of the carrier or other material can depend on the route of administration, e.g., intramuscular, subcutaneous, oral, intravenous, cutaneous, intramucosal (e.g., gut), intranasal or intraperitoneal routes. For liquid injectable preparations, for example, suspensions and solutions, suitable carriers and additives include water, glycols, oils, alcohols, preservatives, coloring agents and the like. For solid oral preparations, for example, powders, capsules, caplets, gelcaps and tablets, suitable carriers and additives include starches, sugars, diluents, granulating agents, lubricants, binders, disintegrating agents and the like. For nasal sprays/inhalant mixtures, the aqueous solution/suspension can comprise water, glycols, oils, emollients, stabilizers, wetting agents, preservatives, aromatics, flavors, and the like as suitable carriers and additives.

Compositions of the invention can be formulated in any matter suitable for administration to a subject to facilitate administration and improve efficacy, including, but not limited to, oral (enteral) administration and parenteral injections. The parenteral injections include intravenous injection or infusion, intra-arterial injection, subcutaneous injection, intramuscular injection, intradermal injection and intra-articular injection. Compositions of the invention can also be formulated for other routes of administration including mucosal (e.g. intravaginal, intranasal, oral, rectal), transmucosal, ocular, rectal, long acting implantation, sublingual administration, under the tongue, from oral mucosa bypassing the portal circulation, inhalation, or intranasal.

Said compositions can be formulated as a vaccine (also referred to as an “immunogenic composition”) according to methods well known in the art. The optimal ratios of each component in the formulation can be determined by techniques well known to those skilled in the art in view of the present disclosure.

In certain embodiments, the vaccine combination as described herein is used in a vaccine regimen that is sometimes referred to as a prime-boost vaccine regimen, in this case being a heterologous prime-boost regimen, which in this context indicates that the priming and boosting vectors are different. In certain embodiments, the priming is with a poxviral vector, such as MVA, and the boosting is with a human adenoviral vector, such as Ad26.

In some embodiments, at least one of the first and second compositions of the invention can further optionally comprise an adjuvant to enhance immune responses. The terms “adjuvant” and “immune stimulant” are used interchangeably herein, and are defined as one or more substances that cause stimulation of the immune system. In this context, an adjuvant is used to enhance an immune response to the human adenovirus and/or poxvirus vectors of the invention.

Adjuvants are defined as substances whose role is to boost or direct antigen specific immune responses when used in combination with specific antigens. Usually, adjuvants combined with antigens, do not induce immune responses against themselves. Due to the poor immunogenic properties of certain antigens, adjuvants are used to enhance, activate and direct the innate and adaptive immune responses to those antigens. The concept of adjuvants has occasionally been extended to carriers that interact with surface molecules on specific cells of the immune system that operate at the interface between the immune system of the host and the administered antigen. In doing so adjuvants help to stimulate the immune system and increase the response to the co-administered antigen. Therefore, adjuvants have been widely used for the development of vaccines.

Preferred adjuvants enhance the immune response against a given antigen by at least a factor of 1.5, 2, 2.5, 5, 10 or 20, as compared to the immune response generated against the antigen under the same conditions but in the absence of the adjuvant. Tests for determining the statistical average enhancement of the immune response against a given antigen as produced by an adjuvant in a group of animals or humans over a corresponding control group are available in the art.

Adjuvants suitable for use with the invention should be ones that are potentially safe, well tolerated and effective in people, such as for instance QS-21, Detox-PC, MPL-SE, MoGM-CSF, TiterMax-G, CRL-1005, GERBU, TERamide, PSC97B, Adjumer, PG-026, GSK-I, GcMAF, B-alethine, MPC-026, Adjuvax, CpG ODN, Betafectin, aluminum salts (e.g. AdjuPhos), Adjuplex, and MF59. The optimal ratios of each component in the formulation can be determined by techniques well known to those skilled in the art in view of the present disclosure.

In preferred embodiments, the first and second compositions of the invention do not comprise an adjuvant.

The preparation and use of immunogenic compositions are well known to those of ordinary skill in the art. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can also be included.

For instance recombinant adenovirus vector may be stored in the buffer that is also used for the Adenovirus World Standard: 20 mM Tris pH 8, 25 mM NaCl, 2.5% glycerol. Another useful adenovirus formulation buffer suitable for administration to humans is 20 mM Tris, 2 mM MgCl₂, 25 mM NaCl, sucrose 10% w/v, polysorbate-80 0.02% w/v. Another formulation buffer that is suitable for recombinant adenovirus comprises 10-25 mM citrate buffer pH 5.9-6.2, 4-6% (w/w) hydroxypropyl-beta-cyclodextrin (HBCD), 70-100 mM NaCl, 0.018-0.035% (w/w) polysorbate-80, and optionally 0.3-0.45% (w/w) ethanol. Obviously, many other buffers can be used, and several examples of suitable formulations for the storage and for pharmaceutical administration of purified vectors are known.

An exemplary preparation and storage of poxviral vectors, including MVA and MVA-BN can be based on the experience in the preparation of poxvirus vaccines used for vaccination against smallpox. Other examples of suitable formulations for poxviral vectors such as MVA are disclosed in WO 2018/211419. Such formulations comprise poxviral vectors such as MVA for instance include but are not limited to compositions comprising 10 mM Tris buffer at pH 8.0 and 100 mM sodium sulfate, or 10 mM phosphate buffer at pH 7.5 and 100 mM sodium sulfate, each of these optionally further comprising 5% (w/w) glycerol.

A subject as used herein preferably is a mammal, or a non-human-primate, or a human. Preferably, the subject is a human subject.

Administration of the first and second composition of the vaccine combination as described herein is typically intramuscular, intradermal or subcutaneous, preferably intramuscular. However, other modes of administration such as intravenous, rectal, vaginal, cutaneous, oral, nasal, etc. can be envisaged as well. Intramuscular administration of the immunogenic compositions can be achieved by using a needle to inject a suspension of the expression vectors, e.g. adenovirus vectors, poxvirus vectors, e.g. into the deltoid muscle of the arm, or vastus lateralis muscle of the thigh, or ventrogluteal muscle of the hip, or in the dorsogluteal muscle of the buttock. An alternative is the use of a needleless injection device to administer the composition (using, e.g., Biojector™) or a freeze-dried powder containing the vaccine.

Unless defined otherwise, all technical and scientific terms used herein have the same meaning commonly understood to one of ordinary skill in the art to which this invention pertains. Otherwise, certain terms cited herein have the meanings as set in the specification. All patents, published patent applications and publications cited herein are incorporated by reference as if set forth fully herein. It must be noted that as used herein and in the appended claims, the singular forms “a,” “an,” and “the” include plural reference unless the context clearly dictates otherwise.

Throughout this description and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the invention can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

The term “protein” or “polypeptide” refers to a molecule consisting of a chain of amino acids, without reference to a specific mode of action, size, 3-dimensional structure or origin. A “fragment” or “portion” of a protein may thus still be referred to as a “protein.” The antigenic protein or immunogenic peptide can be any HIV protein or peptide comprising an epitope (or antigenic determinant). Such antigens can be obtained by sequencing the genomes of the wild-type strains of the different HIV viruses, subcloning the nucleic acids encoding the antigenic determinants from such genomes, and cloning them into the adenoviral genomic sequence and/or the poxviral genomic sequence. Upon administration to a subject, the polypeptide encoded by the nucleic acid molecule in the adenoviral and/or poxviral vectors according to the invention will be expressed in the subject, which will lead to an immune response towards the antigenic fragments that are present in the polypeptide.

“Amino acid sequence”: This refers to the order of amino acid residues of, or within a protein. In other words, any order of amino acids in a protein may be referred to as amino acid sequence.

“Nucleotide sequence”: This refers to the order of nucleotides of, or within a nucleic acid. In other words, any order of nucleotides in a nucleic acid may be referred to as nucleotide sequence.

Methods to insert heterologous coding sequences into a poxviral vector and/or an adenoviral genome are well known to the person skilled in the art. For example, methods for standard molecular biology techniques such as cloning of DNA, DNA and RNA isolation, Western blot analysis, RT-PCR and PCR amplification techniques are described in textbooks and manuals. In a preferred embodiment, the polypeptide encoding the HIV antigen is codon optimized for expression in mammalian cells, preferably human cells. Codon-optimization is a technology well known to the skilled person and widely applied in the art.

Sequence identity is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide.

“Identity” and “similarity” can be readily calculated by known methods. “Sequence identity” and “sequence similarity” can be determined by alignment of two peptide or two nucleotide sequences using global or local alignment algorithms, depending on the length of the two sequences. Sequences of similar lengths are preferably aligned using a global alignment algorithm (e.g. Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blosum62 for proteins and DNAFull for DNA).

Alternatively, percentage similarity or identity may be determined by searching against public databases, using algorithms such as FASTA, BLAST, etc. Thus, the nucleic acid and protein sequences described herein can further be used as a “query sequence” to perform a search against public databases to, for example, identify other family members or related sequences. Such searches can be performed using the BLASTn and BLASTx programs (https://blast.ncbi.nlm.nih.gov/Blast.cgi). BLAST nucleotide searches can be performed with the NBLAST program, score=100, wordlength=12 to obtain nucleotide sequences homologous to nucleic acid molecules of the invention. BLAST protein searches can be performed with the BLASTx program, score=50, word length=3 to obtain amino acid sequences homologous to protein molecules of the invention. To obtain gapped alignments for comparison purposes, Gapped BLAST can also be utilized. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

As used herein, “an effective amount” or “immunologically effective amount” means an amount of a composition sufficient to induce a desired immune effect or immune response in a subject in need thereof. In one embodiment, an effective amount means an amount sufficient to induce an immune response in a subject in need thereof. In another embodiment, an effective amount means an amount sufficient to produce immunity in a subject in need thereof, e.g., provide a protective or therapeutic effect against a disease such as a viral infection. An effective amount can vary depending upon a variety of factors, such as the physical condition of the subject, age, weight, health, etc.; the particular application, whether inducing immune response or providing protective immunity; the specific recombinant vector administered; the immunogen or antigenic polypeptide encoded by the recombinant vector administered; the specific antigenic polypeptide administered; and the particular disease, e.g., viral infection, for which immunity is desired. An effective amount can readily be determined by one of ordinary skill in the art in view of the present disclosure.

As general guidance, an immunogenically effective amount when used with reference to a recombinant viral vector such as an adenoviral vector can be for instance about 10⁸ viral particles to about 10¹² viral particles, for example 10⁸, 10⁹, 10¹⁰, 10¹¹, or 10¹² viral particles. A single dose of adenoviral vectors for administration to humans in certain embodiments is between 10⁹ and 10¹¹ viral particles. An immunogenically effective amount when used with reference to a recombinant viral vector such as a poxviral vector can be for instance about 10⁴ to 10¹¹ TCID₅₀, 10⁵ to 10¹⁰ TCID₅₀, 10⁶ to 10⁹ TCID₅₀, or 10⁷ to 10⁸ TCID₅₀, such as 10⁴, 10⁵, 10⁶, 10⁷, 10⁸, 10⁹, 10¹⁰, or 10¹¹ TCID₅₀. A preferred dose for the subjects (preferably a human) comprises 10⁵ to 10¹⁰ TCID₅₀, such as a dose of 10⁵ TCID₅₀, 10⁶ TCID₅₀, 10⁷ TCID₅₀, 10⁸ TCID₅₀, 10⁹ TCID₅₀, or 10¹⁰ TCID₅₀. The immunogenically effective amount of a poxviral vector such as an MVA vector can alternatively and conveniently be expressed in plaque forming units (pfu), and can for instance be about 10⁵ to about 10¹¹ pfu, e.g. about 10⁵, 10⁶, 10⁷, 10⁸, 10⁹, 10¹⁰ or 10¹¹ pfu, preferably about 10⁷ to 10⁹ pfu, and preferably about 10⁸ pfu, such as for instance about 0.5×10⁸, 1×10⁸, 2×10⁸, 3×10⁸, 4×10⁸, or 5×10⁸ pfu. In certain embodiments, the immunogenically effective amount of an MVA vector according to the invention administered to a human subject is about 1×10⁷ to 1×10⁹ pfu, preferably about 1×10⁸ pfu, preferably in a volume of 0.1 mL to 1 mL, e.g. 0.5 mL.

An immunogenically effective amount of a vector, such as an MVA vector and/or adenovirus vector, can be administered in a single composition, or in multiple compositions, such as 1, 2, 3, 4, 5, 6, 7, 8, 9, or 10 compositions (e.g., tablets, capsules or injectables), wherein the administration of the multiple capsules or injections collectively provides a subject with the immunogenically effective amount. It is also possible to administer an immunogenically effective amount to a subject, and subsequently administer another dose of an immunogenically effective amount to the same subject, in a so-called prime-boost regimen. Further booster administrations can optionally be added to the regimen, as needed.

The medical use herein described is formulated as a composition as defined herein for use as a medicament for treatment of the stated disease(s), but could equally be formulated as a method of treatment of the stated disease(s) using a composition as defined herein, a composition as defined herein for use in the preparation of a medicament to treat the stated disease(s) and use of a composition as defined herein for the treatment of the stated disease(s) by administering an effective amount. Such medical uses are all envisaged by the present invention.

Discussion of documents, acts, materials, devices, articles or the like which has been included in the present specification is for the purpose of providing context for the invention. Such discussion is not an admission that any or all of these matters form part of the prior art with respect to any inventions disclosed or claimed.

The inventors of present application have surprisingly found that when the composition comprising the poxviral vector is administered before administration of the composition comprising the adenoviral vector, antigen-specific T-cell responses and the number of positive peptide pools (giving an indication of the breadth of the response) are significantly increased as compared to other vaccine regimens. Advantageously, these results were already obtained by using a short vaccine regimen (i.e. wherein the time interval between administration of the first vaccine/composition and the second vaccine/composition is less than 6 weeks, preferably even shorter e.g. such a time interval being about 4-25 days, e.g. 10-18 days, e.g. about two weeks).

Accordingly, in certain embodiments the vaccine combination as described herein is for use in inducing a broad T-cell immune response in the subject wherein the broad T-cell immune response is characterized by a significant increase of the number of positive peptide pools as compared to the response obtained in the same assay when the first administration to the subject is with the second composition and the second administration to the subject is with the first composition with the same interval between the first and second administration. Preferably, the vaccine combination as described herein is also for use in inducing a strong T-cell response, which is characterized by a significant increase of immune cells responding to the HIV antigen (i.e. the response magnitude induced against the HIV antigen) in an interferon-gamma ELISpot assay as compared to the response obtained in the same assay when the first administration to the subject is with the second composition and the second administration to the subject is with the first composition with the same interval between the first and second administration.

Additionally, in a second aspect, the invention provides for a method for obtaining a broad T-cell immune response to an HIV antigen in a subject. The method comprises:

i) the administration (‘first administration’) of a first composition comprising a poxvirus vector comprising a polynucleotide that encodes an HIV antigen;
ii) followed less than 6 weeks after the first administration by the administration (‘second administration’) of a second composition comprising a human adenovirus vector comprising a polynucleotide that encodes an HIV antigen,
wherein the broad T-cell immune response is characterized by a significant increase of the number of positive peptide pools as compared to the response obtained when the first administration to the subject is with the second composition and the second administration to the subject is with the first composition under the same conditions (e.g. same time interval between the administrations, same sampling and assay conditions, using the same peptide pools). Preferably, such T-cell response is also a strong T-cell response, which is characterized by a significant increase of immune cells responding to the HIV antigen (i.e. the response magnitude induced against the HIV antigen) in an interferon-gamma ELISpot assay as compared to the response obtained in the same assay when the first administration to the subject is with the second composition and the second administration to the subject is with the first composition with the same interval between the first and second administration. In a preferred embodiment, the second composition is administered less than 6, 5, 4, 3, 2, or 1 week(s) after the administration of the first composition. Preferably, the second composition is administered less than 6 weeks after administration of the first composition, preferably between about 4-25 days, preferably between about 10 to 18 days after administration of the first composition. Preferably, the second composition is administered about 2 weeks (14 days) after administration of the first composition.

As herein described a significant increase in the number of positive peptide pools is an increase of at least 20, 25, 30, 35, 40, 45, 50, 55, 60, or 65% of positive peptide pools as compared to the number of positive peptide pools that are obtained the first administration to the subject is with the second composition and the second administration to the subject is with the first composition.

The method according to the invention provides for a method for obtaining a broad a T-cell immune response to an HIV antigen in a subject. The broad T-cell immune response obtained by the method of the invention may encompass providing protective immunity and/or vaccinating a subject against an HIV antigen for prophylactic purposes, and/or causing a desired immune response or effect in a subject in need thereof against an HIV infection for therapeutic purposes, i.e., therapeutic vaccination. “Inducing an immune response” also encompasses providing a therapeutic immunity for treating against a pathogenic agent. Typically, for prophylactic vaccination, compositions and vaccines are administered to subjects who have not been previously infected with a target pathogen, whereas for therapeutic vaccination, compositions and vaccines are administered to a subject already infected with a target pathogen. CMI breadth (broad T-cell response) is particularly beneficial for therapeutic vaccination, and in certain preferred embodiments the compositions of the invention are used for therapeutic vaccination, e.g. to generate immune responses against HIV in a subject already having an HIV infection.

As described herein a “broad T-cell immune response” is characterized by a significant increase of cell-mediated immunity (CMI) breadth and magnitude. CMI breadth describes the number of epitopic regions in a vaccine antigen recognized by the cellular immune response. CMI magnitude refers to the number of immune cells responding to single peptide or peptide pool stimulation with cytokine secretion, for example measured by IFN-gamma ELISPOT, upon immunization.

CMI breadth can be determined by measuring the positive (response above background) peptide pools against the antigen in an interferon-gamma (IFN-γ) ELISpot assay. The IFN-γ ELISpot assay is well-known to the person skilled in the art; for example, protocols of IFN-γ ELISpot assays have been described in Barouch D H et al (Nature 482.7383 (2012): 89-93) and Barouch D H et al (The Lancet 392.10143 (2018): 232-243).

A non-limiting example of a method for an IFN-γ ELISpot assay and Env peptide pools that can be used to demonstrate a significant increase in the vaccine-induced number of HIV Env positive peptides or peptide pools and/or in the magnitude of the T-cell responses, is the ELISpot assay as described in Barouch et al, The Lancet 392.10143 (2018): 232-243, including the Env peptide pools used therein. In certain embodiments, the number of peptide pools used for one HIV antigen (sometimes also referred to as ‘sub pools’, when belonging to one antigen; herein, the terms ‘pool’ and ‘sub pool’ are used interchangeably) such as Env is at least 10, preferably at least 15, for instance 15, 16, 17, 18, 19, 20, or more. In one particular embodiment the number of Env peptide pools is 17. In certain preferred embodiments peptide pools are potential T-cell epitope (PTE) pools. The peptide pools can be prepared according to known methods, and peptides and/or peptide sequences can for instance be obtained from the NIH AIDS Reagent Program (see e.g. https://www.aidsreagent.org/reagentdetail.cfm?t=peptides&id=330). In one particular embodiment, the Env peptide sequences and allocation to different sub pools in the IFN-γ ELISpot assay described above is provided in Table 2. Thus in certain particular embodiments according to the invention, a T-cell immune response is measured in an interferon-gamma ELISpot assay using 17 Env peptide sub pools, wherein a first sub pool comprises Env peptides having SEQ ID NOs: 13-33, a second sub pool comprises Env peptides having SEQ ID NOs: 34-57, a third sub pool comprises Env peptides having SEQ ID NOs: 58-77, a fourth sub pool comprises Env peptides having SEQ ID NOs: 78-90, a fifth sub pool comprises Env peptides having SEQ ID NOs: 91-110, a sixth sub pool comprises Env peptides having SEQ ID NOs: 111-137, a seventh sub pool comprises Env peptides having SEQ ID NOs: 138-161, an eighth sub pool comprises Env peptides having SEQ ID NOs: 162-180, a ninth sub pool comprises Env peptides having SEQ ID NOs: 181-190, a tenth sub pool comprises Env peptides having SEQ ID NOs: 191-203, an eleventh sub pool comprises Env peptides having SEQ ID NOs: 204-233, a twelfth sub pool comprises Env peptides having SEQ ID NOs: 234-247, a thirteenth sub pool comprises Env peptides having SEQ ID NOs: 248-277, a fourteenth sub pool comprises Env peptides having SEQ ID NOs: 278-300, a fifteenth sub pool comprises Env peptides having SEQ ID NOs: 301-329, a sixteenth sub pool comprises Env peptides having SEQ ID NOs: 330-355, and a seventeenth sub pool comprises Env peptides having SEQ ID NOs: 356-375. Similar assays can be done for other HIV antigens using PTE pools or peptides for such antigens as known to the skilled person, such as Gag, Pol, etc.

CMI magnitude can be determined by counting the number of immune cells responding to single peptide or peptide pool stimulation with cytokine secretion, for example measured by IFN-gamma ELISPOT assay.

Accordingly, the T-cell immune response induced by the method of the invention is characterized by a significant increase of at least one and preferably both of (i) the number and (ii) the magnitude of responses enumerated by the number of responding cells, of positive peptide pools from the antigen in an interferon-gamma ELISpot assay as compared to the response obtained in the same assay when the first administration to the subject is with the second composition and the second administration to the subject is with the first composition under the same conditions (as indicated above).

The first composition according to the second aspect of the invention comprises a poxviral vector as described in the first aspect herein.

The second composition according to the second aspect of the invention comprises a human adenoviral vector as described in the first aspect herein.

The HIV antigen according to the second aspect is as described in the first aspect herein. Preferably, the HIV antigen is Env as described in the first aspect herein.

In a third aspect, the present invention provides for a kit of parts comprising i) a first composition comprising a poxvirus vector as described herein;
ii) a second composition comprising a human adenovirus vector as described herein; and
iii) a manual of instructions detailing that the first composition is to be administered to a subject first and the second composition is to be administered after administration of the first composition and that the time interval between the administration of the first and the second composition is less than 6 weeks e.g. 4-25 days, preferably 10-18 days, preferably wherein the subject suffers from an HIV infection before the first composition is to be administered.

The kit of parts is preferably for a use in treating a HIV infection as described herein.

Optionally, the kit of parts further comprises a leaflet. The leaflet may comprise instructions for use. In addition or alternatively, the leaflet may be at least one of a patient information leaflet and a Summary of Product Characteristics (an SmPC).

In a fourth aspect, the invention provides a first composition comprising a poxvirus vector comprising a polynucleotide that encodes an HIV antigen, for use in combination with a second composition comprising a human adenovirus vector comprising a polynucleotide that encodes an HIV antigen, wherein the first composition is for administration to the subject and the second composition is for administration to the subject less than six weeks after administration of the first composition.

In a fifth aspect, the invention provides a second composition comprising a human adenovirus vector comprising a polynucleotide that encodes an HIV antigen, for use in combination with a first composition comprising a poxvirus vector comprising a polynucleotide that encodes an HIV antigen, wherein the first composition is for administration to the subject and the second composition is for administration to the subject less than six weeks after administration of the first composition.

The first and second composition, the vectors, timing of administration, and the HIV antigen, can all be varied and are preferred also for the fifth and sixth aspects of the invention according to embodiments of the invention as described in more detail above for the first and second aspects.

### EXAMPLES

The following examples of the invention are to further illustrate the nature of the invention. It should be understood that the following examples do not limit the invention and that the scope of the invention is to be determined by the appended claims.

Cell-mediated immunity (CMI) breadth describes the number of epitopic regions in a vaccine antigen recognized by the cellular immune response. In non-human primate studies, CMI breadth has been identified as an important correlate of efficacy. To determine if CMI breadth could be optimized by modifying the immunization regimen, e.g. order of administration of vaccine components and timing of administration, vaccine regimens were compared as described below.

### Example 1: MVA-Ad Immunization Regimen Induces Significantly Broader and Stronger Immune Response than Ad-MVA Regimen

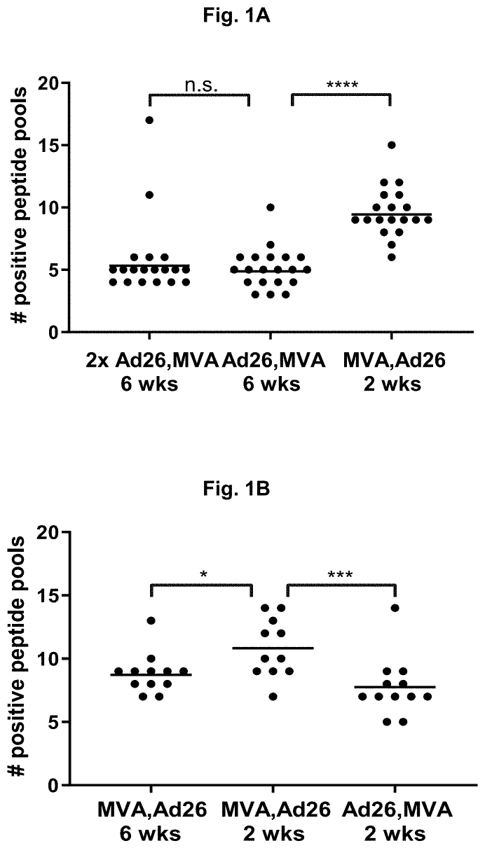

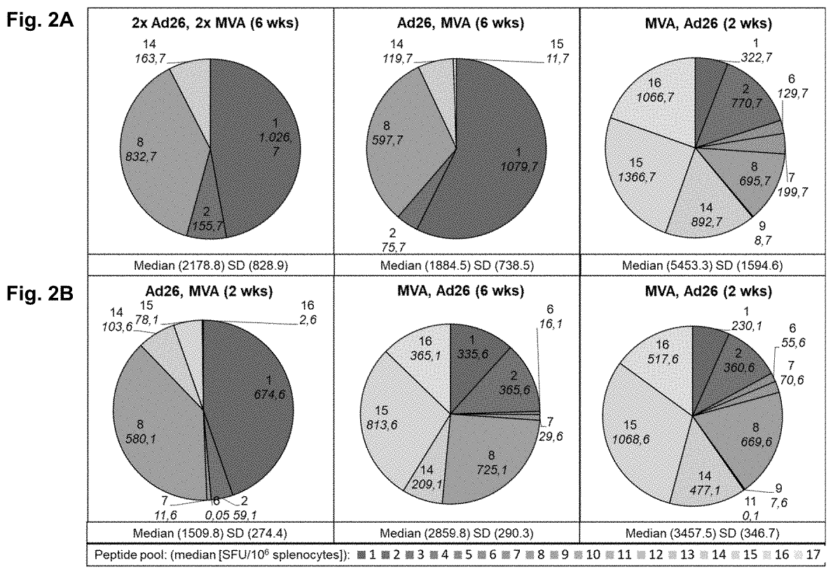

Female CB6/F1 mice, 6-8 weeks old, received intramuscular immunizations with MVA-BN-HIV (MVA vector encoding Mos1.GagPol, Mos2.GagPol, Mos1.Env, and Mos2S.Env, for instance described in WO 2018/229711, referred to therein as ‘MVA-mBN414’; dosed at a total of 2×10⁷ TCID₅O per complete regimen either split between 2 administrations or in a single administration, see Brief description of the drawings for FIGS. 1A, 1B, 2A, and 2B) and Ad26.Mos4.HIV (composition comprising four Ad26 vectors in equimolar ratio, respectively encoding Mos1.GagPol, Mos2.GagPol, Mos1.Env, and Mos2S.Env, which composition is for instance described in example 6 of WO 2017/102929; dosed at a total of 5×10⁹ viral particles per complete regimen either split between 2 administrations or in a single administration, see Brief description of the drawings for FIGS. 1A, 1B, 2A, and 2B) in the hind quadricep muscles at a volume of 50 μL/leg and administered in a 2 or 6-week interval. To examine the effect of order of the vectors, additional groups were included that received Ad26.Mos4.HIV as first dose(s) and MVA-BN-HIV as follow-up dose(s). Two weeks following the final dose, mice were sacrificed and splenocytes were collected for analysis of cellular immune responses using IFN-γ ELISpot.

Briefly, IFN-γ ELISpot was performed on splenocytes of mice isolated after sacrifice using mouse IFN-γ ELISpot-plus kit (Mabtech). Splenocytes were obtained by disaggregation of spleens with the gentleMACS (Miltenyibiotech) dissociator. IFN-γ ELISpot assay was performed by stimulating splenocytes from individual mice for 18 h with 17 different HIV PTE Env peptide pools (see Table 2 for sequences of peptides and allocation to the sub pools) at a final concentration of 1 μg/peptide/mL. PMA/ionomycin stimulation was used as a positive control; the data were gathered for information purposes only. Medium was used as negative control (background) and used to calculate the lower limit of detection (less than 50 spots/10⁶ cells expected). Stimulation was done overnight in duplicate wells containing 5×10⁵ cells per well.

To analyze CMI breadth, splenocytes were stimulated overnight with 17 peptide pools (see Table 2) longitudinally covering the HIV Envelope protein and designed to cover potential human T cell epitopes (PTE) of circulating HIV-1 strains. Medium was used as a negative control and the lower limit of detection was set at 50 spots/10⁶ cells. A positive response to a peptide pool was defined as a spot count above background.

FIGS. 1A and 1B represent two independent experiments.

As can be seen in FIG. 1, a very short regimen of MVA-BN-HIV at week 0 followed by immunization with Ad26.Mos4.HIV at week 2 (abbreviated MVA, Ad26) induced a response to the highest number of pools compared to other tested regimens. Each pool covers a part of the Env protein of HIV and therefore more positive peptide pools translates into increased cellular breadth as the responses of these animals are directed to a broader range of epitopes. The MVA, Ad26 regimen induced responses to significantly more peptide pools than regimens using the reversed order of vector application and longer intervals between immunizations (FIG. 1A). FIG. 1B shows that MVA, Ad26 with a 2-week interval between immunizations induced responses to significantly more peptide pools than (1) the same regimen with longer interval and (2) a reversed regimen with the same interval, showing that both order of vectors and time between immunizations influence the outcome.

Higher breadth also correlated with higher magnitude (FIG. 2), where each gray tone refers to a different peptide pool and median spot forming units are depicted per pool. The regimens where Ad26.Mos4.HIV was used as a first dose, or first two doses in the 4-dose immunization regimen, induced lowest breadth with responses to 4-8 peptide pools. In contrast, regimens in which MVA-BN-HIV was administered first induced responses to 8-10 peptide pools. The regimen with MVA-BN-HIV at week 0 and Ad26.Mos4.HIV at week 2 induced the greatest CMI breadth and magnitude in both studies.

FIGS. 2A and 2B represent two independent experiments (the experiment in FIG. 2A is the same as the experiment for which data are shown in FIG. 1A, while the experiment in FIG. 2B is the same as the experiment for which data are shown in FIG. 1B).

The results described for the experiments above show that a regimen consisting of immunization with MVA-BN-HIV followed by Ad26.Mos4.HIV showed significantly higher CMI breadth and magnitude compared to other tested vaccine regimens with different order or number of immunizations. Furthermore, these effects were already observed in the regimen wherein MVA-BN-HIV is administered at week 0 and wherein Ad26.Mos4.HIV is administered two weeks after the first administration. The fact that only a short time span between doses is sufficient to boost this response is especially beneficial in a clinical setting where it may simplify vaccine dosing regimens, and for instance increase compliance to the complete multiple dosing regimens. These results seem to be specific for HIV antigens as such an increase in CMI breadth and magnitude was not observed with other viral antigens (data not shown).

TABLE 1
Exemplary mosaic HIV antigen sequences

mos1.Gag polypeptide (SEQ ID NO: 1)
MGARASVLSGGELDRWEKIRLRPGGKKKYRLKHIVWASRELERFAVNPGLLETSEGCRQILGQLQPSLQTGSEELRSLYN
TVATLYCVHQRIEIKDTKEALEKIEEEQNKSKKKAQQAAADTGNSSQVSQNYPIVQNIQGQMVHQAISPRTLNAWVKVVE
EKAFSPEVIPMFSALSEGATPQDLNTMLNTVGGHQAAMQMLKETINEEAAEWDRVHPVHAGPIAPGQMREPRGSDIAGTT
STLQEQIGWMTNNPPIPVGEIYKRWIILGLNKIVRMYSPVSILDIRQGPKEPFRDYVDRFYKTLRAEQASQDVKNWMTET
LLVQNANPDCKTILKALGPAATLEEMMTACQGVGGPGHKARVLAEAMSQVTNSATIMMQRGNFRNQRKTVKCFNCGKEGH
IAKNCRAPRKKGCWKCGKEGHQMKDCTERQANFLGKIWPSNKGRPGNFLQNRPEPTAPPEESFRFGEETTTPSQKQEPID
KEMYPLASLKSLFGNDPSSQ
mos2.Gag polypeptide (SEQ ID NO: 2)
MGARASILRGGKLDKWEKIRLRPGGKKHYMLKHLVWASRELERFALNPGLLETSEGCKQIIKQLQPALQTGTEELRSLFN
TVATLYCVHAEIEVRDTKEALDKIEEEQNKSQQKTQQAKEADGKVSQNYPIVQNLQGQMVHQPISPRTLNAWVKVIEEKA
FSPEVIPMFTALSEGATPQDLNTMLNTVGGHQAAMQMLKDTINEEAAEWDRLHPVHAGPVAPGQMREPRGSDIAGTTSNL
QEQIAWMTSNPPIPVGDIYKRWIILGLNKIVRMYSPTSILDIKQGPKEPFRDYVDRFFKTLRAEQATQDVKNWMTDTLLV
QNANPDCKTILRALGPGATLEEMMTACQGVGGPSHKARVLAEAMSQTNSTILMQRSNFKGSKRIVKCFNCGKEGHIARNC
RAPRKKGCWKCGKEGHQMKDCTERQANFLGKIWPSHKGRPGNFLQSRPEPTAPPAESFRFEETTPAPKQEPKDREPLTSL
RSLFGSDPLSQ
mos1.Pol polypeptide (SEQ ID NO: 3)
MAPISPIETVPVKLKPGMDGPRVKQWPLTEEKIKALTAICEEMEKEGKITKIGPENPYNTPVFAIKKKDSTKWRKLVDFR
ELNKRTQDFWEVQLGIPHPAGLKKKKSVTVLAVGDAYFSVPLDEGFRKYTAFTIPSTNNETPGIRYQYNVLPQGWKGSPA
IFQCSMTRILEPFRAKNPEIVIYQYMAALYVGSDLEIGQHRAKIEELREHLLKWGFTTPDKKHQKEPPFLWMGYELHPDK
WTVQPIQLPEKDSWTVNDIQKLVGKLNWASQIYPGIKVRQLCKLLRGAKALTDIVPLTEEAELELAENREILKEPVHGVY
YDPSKDLIAEIQKQGHDQWTYQIYQEPFKNLKTGKYAKMRTAHTNDVKQLTEAVQKIAMESIVIWGKTPKFRLPIQKETW
ETWWTDYWQATWIPEWEFVNTPPLVKLWYQLEKDPIAGVETFYVAGAANRETKLGKAGYVTDRGRQKIVSLTETTNQKTA
LQAIYLALQDSGSEVNIVTASQYALGIIQAQPDKSESELVNQIIEQLIKKERVYLSWVPAHKGIGGNEQVDKLVSSGIRK
VLFLDGIDKAQEEHEKYHSNWRAMASDFNLPPVVAKEIVASCDQCQLKGEAMHGQVDCSPGIWQLACTHLEGKIILVAVH
VASGYIEAEVIPAETGQETAYFILKLAGRWPVKVIHTANGSNFTSAAVKAACWWAGIQQEFGIPYNPQSQGVVASMNKEL
KKIIGQVRDQAEHLKTAVQMAVFIHNFKRKGGIGGYSAGERIIDIIATDIQTKELQKQIIKIQNFRVYYRDSRDPIWKGP
AKLLWKGEGAVVIQDNSDIKVVPRRKVKIIKDYGKQMAGADCVAGRQDED
mos2.Pol polypeptide (SEQ ID NO: 4)
MAPISPIETVPVKLKPGMDGPKVKQWPLTEEKIKALVEICTEMEKEGKISKIGPENPYNTPIFAIKKKDSTKWRKLVDFR
ELNKRTQDFWEVQLGIPHPAGLKKKKSVTVLAVGDAYFSVPLDEDFRKYTAFTIPSINNETPGIRYQYNVLPQGWKGSPA
IFQSSMTKILEPFRKQNPDIVIYQYMAALYVGSDLEIGQHRTKIEELRQHLLRWGFTTPDKKHQKEPPFLWMGYELHPDK
WTVQPIVLPEKDSWTVNDIQKLVGKLNWASQIYAGIKVKQLCKLLRGTKALTEVVPLTEEAELELAENREILKEPVHGVY
YDPSKDLIAEIQKQGQGQWTYQIYQEPFKNLKTGKYARMRGAHTNDVKQLTEAVQKIATESIVIWGKTPKFKLPIQKETW
EAWWTEYWQATWIPEWEFVNTPPLVKLWYQLEKEPIVGAETFYVAGAANRETKLGKAGYVTDRGRQKVVSLTDTTNQKTA
LQAIHLALQDSGLEVNIVTASQYALGIIQAQPDKSESELVSQIIEQLIKKEKVYLAWVPAHKGIGGNEQVDKLVSRGIRK
VLFLDGIDKAQEEHEKYHSNWRAMASEFNLPPIVAKEIVASCDKCQLKGEAIHGQVDCSPGIWQLACTHLEGKVILVAVH
VASGYIEAEVIPAETGQETAYFLLKLAGRWPVKTIHTANGSNFTSATVKAACWWAGIKQEFGIPYNPQSQGVVASINKEL
KKIIGQVRDQAEHLKTAVQMAVFIHNFKRKGGIGEYSAGERIVDIIASDIQTKELQKQITKIQNFRVYYRDSRDPLWKGP
AKLLWKGEGAVVIQDNSDIKVVPRRKAKIIRDYGKQMAGDDCVASRQDED
mos1.Env polypeptide (SEQ ID NO: 5)
MRVTGIRKNYQHLWRWGTMLLGILMICSAAGKLWVTVYYGVPVWKEATTTLFCASDAKAYDTEVHNVWATHACVPTDPNP
QEVVLENVTENFNMWKNNMVEQMHEDIISLWDQSLKPCVKLTPLCVTLNCTDDVRNVTNNATNTNSSWGEPMEKGEIKNC
SFNITTSIRNKVQKQYALFYKLDVVPIDNDSNNTNYRLISCNTSVITQACPKVSFEPIPIHYCAPAGFAILKCNDKKFNG
TGPCTNVSTVQCTHGIRPVVSTQLLLNGSLAEEEVVIRSENFTNNAKTIMVQLNVSVEINCTRPNNNTRKSIHIGPGRAF
YTAGDIIGDIRQAHCNISRANWNNTLRQIVEKLGKQFGNNKTIVFNHSSGGDPEIVMHSFNCGGEFFYCNSTKLFNSTWT
WNNSTWNNTKRSNDTEEHITLPCRIKQIINMWQEVGKAMYAPPIRGQIRCSSNITGLLLTRDGGNDTSGTEIFRPGGGDM
RDNWRSELYKYKVVKIEPLGVAPTKAKRRVVQSEKSAVGIGAVFLGFLGAAGSTMGAASMTLTVQARLLLSGIVQQQNNL
LRAIEAQQHLLQLTVWGIKQLQARVLAVERYLKDQQLLGIWGCSGKLICTTTVPWNASWSNKSLDKIWNNMTWMEWEREI
NNYTSLIYTLIEESQNQQEKNEQELLELDKWASLWNWFDISNWLW
mos2S.Env polypeptide (SEQ ID NO: 6)
MRVRGMLRNWQQWWIWSSLGFWMLMIYSVMGNLWVTVYYGVPVWKDAKTTLFCASDAKAYEKEVHNVWATHACVPTDPNP
QEIVLGNVTENFNMWKNDMVDQMHEDIISLWDASLEPCVKLTPLCVTLNCRNVRNVSSNGTYNIIHNETYKEMKNCSFNA
TTVVEDRKQKVHALFYRLDIVPLDENNSSEKSSENSSEYYRLINCNTSAITQACPKVSFDPIPIHYCAPAGYAILKCNNK
TFNGTGPCNNVSTVQCTHGIKPVVSTQLLLNGSLAEEEIIIRSENLTNNAKTIIVHLNETVNITCTRPNNNTRKSIRIGP
GQTFYATGDIIGDIRQAHCNLSRDGWNKTLQGVKKKLAEHFPNKTIKFAPHSGGDLEITTHTFNCRGEFFYCNTSNLFNE
SNIERNDSIITLPCRIKQIINMWQEVGRAIYAPPIAGNITCRSNITGLLLTRDGGSNNGVPNDTETFRPGGGDMRNNWRS
ELYKYKVVEVKPLGVAPTEAKRRVVEREKRAVGIGAVFLGILGAAGSTMGAASITLTVQARQLLSGIVQQQSNLLRAIEA
QQHMLQLTVWGIKQLQTRVLAIERYLQDQQLLGLWGCSGKLICTTAVPWNTSWSNKSQTDIWDNMTWMQWDKEIGNYTGE
IYRLLEESQNQQEKNEKDLLALDSWNNLWNWFSISKWLWYIKIFIMIVGGLIGLRIIFAVLSIVNRVRQGY
nucleotide sequence encoding mos1.GagPol (SEQ ID NO: 7)
ATGGGAGCCAGAGCCAGCGTGCTGTCCGGAGGGGAGCTGGACCGCTGGGAGAAGATCAGGCTGAGGCCTGGAGGGAAGAA
GAAGTACAGGCTGAAGCACATCGTGTGGGCCAGCAGAGAGCTGGAACGGTTTGCCGTGAACCCTGGCCTGCTGGAAACCA
GCGAGGGCTGTAGGCAGATTCTGGGACAGCTGCAGCCCAGCCTGCAGACAGGCAGCGAGGAACTGCGGAGCCTGTACAAC
ACCGTGGCCACCCTGTACTGCGTGCACCAGCGGATCGAGATCAAGGACACCAAAGAAGCCCTGGAAAAGATCGAGGAAGA
GCAGAACAAGAGCAAGAAGAAAGCCCAGCAGGCTGCCGCTGACACAGGCAACAGCAGCCAGGTGTCCCAGAACTACCCCA
TCGTGCAGAACATCCAGGGACAGATGGTGCACCAGGCCATCAGCCCTCGGACCCTGAACGCCTGGGTGAAGGTGGTGGAG
GAAAAGGCCTTCAGCCCTGAGGTGATCCCCATGTTCTCTGCCCTGAGCGAGGGAGCCACACCCCAGGACCTGAACACCAT
GCTGAACACCGTGGGAGGGCACCAGGCTGCCATGCAGATGCTGAAAGAGACAATCAACGAGGAAGCTGCCGAGTGGGACA
GGGTCCACCCAGTGCACGCTGGACCTATCGCTCCTGGCCAGATGAGAGAGCCCAGAGGCAGCGATATTGCTGGCACCACC
TCCACACTGCAGGAACAGATCGGCTGGATGACCAACAACCCTCCCATCCCTGTGGGAGAGATCTACAAGCGGTGGATCAT
TCTGGGACTGAACAAGATCGTGCGGATGTACAGCCCTGTGAGCATCCTGGACATCAGGCAGGGACCCAAAGAGCCCTTCA
GGGACTACGTGGACCGGTTCTACAAGACCCTGAGAGCCGAGCAGGCCAGCCAGGACGTGAAGAACTGGATGACCGAGACA
CTGCTGGTGCAGAACGCCAACCCTGACTGCAAGACCATCCTGAAAGCCCTGGGACCTGCTGCCACCCTGGAAGAGATGAT
GACAGCCTGCCAGGGAGTGGGAGGACCTGGCCACAAGGCCAGGGTGCTGGCCGAGGCCATGAGCCAGGTGACCAACTCTG
CCACCATCATGATGCAGAGAGGCAACTTCCGGAACCAGAGAAAGACCGTGAAGTGCTTCAACTGTGGCAAAGAGGGACAC
ATTGCCAAGAACTGCAGGGCTCCCAGGAAGAAAGGCTGCTGGAAGTGCGGAAAAGAAGGCCACCAGATGAAGGACTGCAC
CGAGAGGCAGGCCAACTTCCTGGGCAAGATCTGGCCTAGCAACAAGGGCAGGCCTGGCAACTTCCTGCAGAACAGACCCG
AGCCCACCGCTCCTCCCGAGGAAAGCTTCCGGTTTGGCGAGGAAACCACCACCCCTAGCCAGAAGCAGGAACCCATCGAC
AAAGAGATGTACCCTCTGGCCAGCCTGAAGAGCCTGTTCGGCAACGACCCCAGCAGCCAGATGGCTCCCATCAGCCCAAT
CGAGACAGTGCCTGTGAAGCTGAAGCCTGGCATGGACGGACCCAGGGTGAAGCAGTGGCCTCTGACCGAGGAAAAGATCA
AAGCCCTGACAGCCATCTGCGAGGAAATGGAAAAAGAGGGCAAGATCACCAAGATCGGACCCGAGAACCCCTACAACACC
CCTGTGTTCGCCATCAAGAAGAAAGACAGCACCAAGTGGAGGAAACTGGTGGACTTCAGAGAGCTGAACAAGCGGACCCA
GGACTTCTGGGAGGTGCAGCTGGGCATCCCTCACCCTGCTGGCCTGAAGAAAAAGAAAAGCGTGACCGTGCTGGCTGTGG
GAGATGCCTACTTCAGCGTGCCTCTGGACGAGGGCTTCCGGAAGTACACAGCCTTCACCATCCCCAGCACCAACAACGAG
ACACCTGGCATCAGATACCAGTACAACGTGCTGCCTCAGGGCTGGAAAGGCAGCCCTGCCATCTTCCAGTGCAGCATGAC
CAGAATCCTGGAACCCTTCAGAGCCAAGAACCCTGAGATCGTGATCTACCAGTATATGGCTGCCCTCTACGTGGGCAGCG
ACCTGGAAATCGGACAGCACAGAGCCAAAATCGAAGAACTCCGCGAGCACCTGCTGAAGTGGGGATTCACCACCCCTGAC
AAGAAGCACCAGAAAGAGCCTCCCTTCCTGTGGATGGGCTACGAGCTGCACCCTGACAAGTGGACCGTGCAGCCCATCCA
GCTGCCAGAGAAGGACTCCTGGACCGTGAACGACATCCAGAAACTGGTCGGCAAGCTGAACTGGGCCAGCCAGATCTACC
CTGGCATCAAAGTCAGACAGCTGTGTAAGCTGCTGAGGGGAGCCAAAGCACTGACCGACATCGTGCCTCTGACAGAAGAA
GCCGAGCTGGAACTGGCCGAGAACAGAGAGATCCTGAAAGAACCCGTGCACGGAGTGTACTACGACCCCTCCAAGGACCT
GATTGCCGAGATCCAGAAACAGGGACACGACCAGTGGACCTACCAGATCTATCAGGAACCTTTCAAGAACCTGAAAACAG
GCAAGTACGCCAAGATGCGGACAGCCCACAOCAACGACGTGAAGCAGCTGACCGAAGCCGTGCAGAAAATCGCCATGGAA
AGCATCGTGATCTGGGGAAAGACACCCAAGTTCAGGCTGCCCATCCAGAAAGAGACATGGGAAACCTGGTGGACCGACTA
CTGGCAGGCCACCTGGATTCCCGAGTGGGAGTTCGTGAACACCCCACCCCTGGTGAAGCTGTGGTATCAGCTGGAAAAGG
ACCCTATCGCTGGCGTGGAGACATTCTACGTGGCTGGAGCTGCCAACAGAGAGACAAAGCTGGGCAAGGCTGGCTACGTG
ACCGACAGAGGCAGACAGAAAATCGTGAGCCTGACCGAAACCACCAACCAGAAAACAGCCCTGCAGGCCATCTATCTGGC
ACTGCAGGACAGCGGAAGCGAGGTGAACATCGTGACAGCCAGCCAGTATGCCCTGGGCATCATCCAGGCCCAGCCTGACA
AGAGCGAGAGCGAGCTGGTGAACCAGATCATCGAGCAGCTGATCAAGAAAGAACGGGTGTACCTGAGCTGGGTGCCAGCC
CACAAGGGCATCGGAGGGAACGAGCAGGTGGACAAGCTGGTGTCCAGCGGAATCCGGAAGGTGCTGTTCCTGGACGGCAT
CGATAAAGCCCAGGAAGAGCACGAGAAGTACCACAGCAATTGGAGAGCCATGGCCAGCGACTTCAACCTGCCTCCCGTGG
TGGCCAAAGAAATCGTGGCCAGCTGCGACCAGTGCCAGCTGAAAGGCGAGGCCATGCACGGACAGGTGGACTGCTCCCCT
GGCATCTGGCAGCTGGCATGCACCCACCTGGAAGGCAAGATCATTCTGGTGGCCGTGCACGTGGCCAGCGGATACATCGA
AGCCGAAGTGATCCCTGCCGAGACAGGGCAGGAAACAGCCTACTTCATCCTGAAGCTGGCTGGCAGATGGCCTGTGAAGG
TGATCCACACAGCCAACGGCAGCAACTTCACCTCTGCTGCCGTGAAGGCTGCCTGTTGGTGGGCTGGCATTCAGCAGGAA
TTTGGCATCCCCTACAATCCCCAGTCTCAGGGAGTGGTGGCCAGCATGAACAAAGAGCTGAAGAAGATCATCGGACAGGT
CAGGGATCAGGCCGAGCACCTGAAAACTGCCGTCCAGATGGCCGTGTTCATCCACAACTTCAAGCGGAAGGGAGGGATCG
GAGGGTACTCTGCTGGCGAGCGGATCATCGACATCATTGCCACCGATATCCAGACCAAAGAGCTGCAGAAACAGATCATC
AAGATCCAGAACTTCAGGGTGTACTACAGGGACAGCAGGGACCCCATCTGGAAGGGACCTGCCAAGCTGOTGTGGAAAGG
CGAAGGAGCCGTCGTCATCCAGGACAACAGCGACATCAAGGTGGTGCCCAGACGGAAGGTGAAAATCATCAAGGACTACG
GCAAACAGATGGCTGGAGCCGACTGTGTCGCTGGCAGGCAGGACGAGGAC
nucleotide sequence encoding mos2.GagPol (SEQ ID NO: 8)
ATGGGAGCCAGAGCCAGCATCCTGCGAGGAGGGAAGCTGGACAAGTGGGAGAAGATCAGGCTGAGGCCTGGAGGGAAGAA
ACACTACATGCTGAAGCACCTGGTCTGGGCCAGCAGAGAGCTGGAACGGTTTGCCCTCAATCCTGGCCTGCTGGAAACCA
GCGAGGGCTGCAAGCAGATCATCAAGCAGCTGCAGCCTGCCCTGCAGACAGGCACCGAGGAACTGCGGAGCCTGTTCAAC
ACCGTGGCCACCCTGTACTGCGTGCATGCCGAGATCGAAGTGAGGGACACCAAAGAAGCCCTGGACAAGATCGAGGAAGA
GCAGAACAAGAGCCAGCAGAAAACCCAGCAGGCCAAAGAAGCCGACGGCAAGGTCTCCCAGAACTACCCCATCGTGCAGA
ACCTGCAGGGACAGATGGTGCACCAGCCCATCAGCCCTCGGACACTGAATGCCTGGGTGAAGGTGATCGAGGAAAAGGCC
TTCAGCCCTGAGGTGATCCCCATGTTCACAGCCCTGAGCGAGGGAGCCACACCCCAGGACCTGAACACCATGCTGAACAC
CGTGGGAGGGCACCAGGCTGCCATGCAGATGCTGAAGGACACCATCAACGAGGAAGCTGCCGAGTGGGACAGGCTGCACC
CTGTGCACGCTGGACCTGTGGCTCCTGGCCAGATGAGAGAGCCCAGAGGCAGCGATATTGCTGGCACCACCTCCAATCTG
CAGGAACAGATCGCCTGGATGACCAGCAACCCTCCCATCCCTGTGGGAGACATCTACAAGCGGTGGATCATCCTGGGACT
GAACAAGATCGTGCGGATGTACAGCCCTACCTCCATCCTGGACATCAAGCAGGGACCCAAAGAGCCTTTCAGGGACTACG
TGGACCGGTTCTTCAAGACCCTGAGAGCCGAGCAGGCCACCCAGGACGTGAAGAACTGGATGACCGACACCCTGCTGGTG
CAGAACGCCAACCCTGACTGCAAGACCATCCTGAGAGCCCTGGGACCTGGAGCCACCCTGGAAGAGATGATGACAGCCTG
CCAGGGAGTGGGAGGACCCTCTCACAAGGCTAGGGTGCTGGCCGAGGCCATGAGCCAGACCAACAGCACCATCCTGATGC
AGCGGAGCAACTTCAAGGGCAGCAAGCGGATCGTGAAGTGCTTCAACTGTGGCAAAGAGGGACACATTGCCAGAAACTGT
AGGGCACCCAGGAAGAAAGGCTGCTGGAAGTGCGGAAAAGAAGGCCACCAGATGAAGGACTGCACCGAGAGGCAGGCCAA
CTTCCTGGGCAAGATCTGGCCTAGCCACAAGGGCAGACCTGGCAACTTCCTGCAGAGCAGACCCGAGCCCACCGCTCCTC
CAGCCGAGAGCTTCCGGTTCGAGGAAACCACCCCTGCTCCCAAGCAGGAACCTAAGGACAGAGAGCCTCTGACCAGCCTG
AGAAGCCTGTTCGGCAGCGACCCTCTGAGCCAGATGGCTCCCATCTCCCCTATCGAGACAGTGCCTGTGAAGCTGAAGCC
TGGCATGGACGGACCCAAGGTGAAACAGTGGCCTCTGACCGAGGAAAAGATCAAAGCCCTGGTGGAGATCTGTACCGAGA
TGGAAAAAGAGGGCAAGATCAGCAAGATCGGACCCGAGAACCCCTACAACACCCCTATCTTCGCCATCAAGAAGAAAGAC
AGCACCAAGTGGAGGAAACTGGTGGACTTCAGAGAGCTGAACAAGCGGACCCAGGACTTCTGGGAGGTGCAGCTGGGCAT
CCCTCACCCTGCTGGCCTGAAGAAAAAGAAAAGCGTGACCGTGCTGGCCGTGGGAGATGCCTACTTCAGCGTGCCTCTGG
ACGAGGACTTCAGAAAGTACACAGCCTTCACCATCCCCAGCATCAACAACGAGACACCTGGCATCAGATACCAGTACAAC
GTGCTGCCTCAGGGATGGAAGGGCTCTCCTGCAATCTTCCAGAGCAGCATGACCAAGATCCTGGAACCCTTCCGGAAGCA
GAACCCTGACATCGTGATCTACCAGTACATGGCAGCCCTGTACGTCGGCAGCGACCTGGAAATCGGACAGCACCGGACCA
AGATCGAAGAACTCAGGCAGCACCTGCTGCGGTGGGGATTCACCACCCCTGACAAGAAGCACCAGAAAGAGCCTCCCTTC
CTGTGGATGGGCTACGAGCTGCACCCAGACAAGTGGACCGTGCAGCCCATCGTGCTGCCTGAGAAGGACTCCTGGACCGT
GAACGACATCCAGAAACTGGTCGGCAAGCTGAACTGGGCCAGCCAGATCTACGCTGGCATCAAAGTGAAGCAGCTGTGTA
AGCTCCTGAGAGGCACCAAAGCCCTGACCGAGGTGGTGCCACTGACAGAGGAAGCCGAGCTGGAACTGGCCGAGAACAGA
GAGATCCTGAAAGAACCCGTGCACGGAGTGTACTACGACCCCAGCAAGGACCTGATTGCCGAGATCCAGAAGCAGGGACA
GGGACAGTGGACCTACCAGATCTACCAGGAACCCTTCAAGAACCTGAAAACAGGCAAGTACGCCAGGATGAGGGGAGCCC
ACACCAACGACGTCAAACAGCTGACCGAAGCCGTGCAGAAGATCGCCACCGAGAGCATCGTGATTTGGGGAAAGACACCC
AAGTTCAAGCTGCCCATCCAGAAAGAGACATGGGAGGCCTGGTGGACCGAGTACTGGCAGGCCACCTGGATTCCCGAGTG
GGAGTTCGTGAACACCCCACCCCTGGTGAAGCTGTGGTATCAGCTGGAAAAAGAACCCATCGTGGGAGCCGAGACATTCT
ACGTGGCTGGAGCTGCCAACAGAGAGACAAAGCTGGGCAAGGCTGGCTACGTGACCGACAGAGGCAGGCAGAAAGTGGTG
TCCCTGACCGATACCACCAACCAGAAAACAGCCCTGCAGGCCATCCACCTGGCTCTGCAGGACTCTGGCCTGGAAGTGAA
CATCGTGACAGCCAGCCAGTATGCCCTGGGCATCATTCAGGCACAGCCTGACAAGAGCGAGAGCGAGCTGGTGTCTCAGA
TCATTGAGCAGCTGATCAAGAAAGAAAAGGTGTACCTGGCCTGGGTGCCAGCCCACAAGGGGATCGGAGGGAACGAGCAG
GTGGACAAGCTGGTGTCCAGGGGCATCCGGAAGGTGCTGTTTCTGGACGGCATCGACAAAGCCCAGGAAGAGCACGAGAA
GTACCACAGCAATTGGAGAGCCATGGCCAGCGAGTTCAACCTGCCTCCCATCGTGGCCAAAGAAATCGTGGCCTCTTGCG
ACAAGTGCCAGCTGAAAGGCGAGGCCATTCACGGACAGGTGGACTGCAGCCCAGGCATCTGGCAGCTGGCCTGCACCCAC
CTGGAAGGCAAGGTGATCCTGGTGGCCGTGCACGTGGCCTCTGGATACATCGAAGCCGAAGTGATCCCTGCCGAGACAGG
CCAGGAAACAGCCTACTTCCTGCTGAAGCTGGCTGGCAGGTGGCCTGTGAAAACCATCCACACAGCCAACGGCAGCAACT
TCACCTCTGCCACCGTGAAGGCTGCCTGTTGGTGGGCTGGCATTAAGCAGGAATTTGGCATCCCCTACAACCCTCAGTCT
CAGGGAGTGGTGGCCTCCATCAACAAAGAGCTGAAGAAGATCATCGGACAGGTCAGGGATCAGGCCGAGCATCTGAAAAC
AGCCGTCCAGATGGCCGTGTTCATCCACAACTTCAAGCGGAAGGGAGGGATCGGAGAGTACTCTGCTGGCGAGAGGATCG
TGGACATTATCGCCAGCGATATCCAGACCAAAGAACTGCAGAAGCAGATCACAAAGATCCAGAACTTCAGGGTGTACTAC
AGGGACAGCAGAGATCCCCTGTGGAAGGGACCTGCCAAGCTGCTGTGGAAAGGCGAAGGAGCCGTCGTCATCCAGGACAA
CAGCGACATCAAGGTGGTGCCCAGACGGAAGGCCAAGATCATCAGAGACTACGGCAAACAGATGGCTGGCGACGACTGCG
TCGCCTCTAGGCAGGACGAGGAC
nucleotide sequence encoding mos1.Env (SEQ ID NO: 9)
ATGCGGGTGACCGGCATCCGGAAGAACTACCAGCACCTGTGGCGGTGGGGCACCATGCTGCTGGGCATCCTGATGATTTG
CTCTGCCGCCGGAAAGCTGTGGGTGACCGTGTACTACGGCGTGCCCGTGTGGAAAGAGGCCACCACCACCCTGTTCTGCG
CCAGCGACGCCAAGGCCTACGACACCGAGGTGCACAACGTGTGGGCCACCCACGCCTGCGTGCCCACCGACCCCAACCCC
CAGGAAGTGGTCCTGGAAAACGTGACCGAGAACTTCAACATGTGGAAGAACAACATGGTGGAGCAGATGCACGAGGACAT
CATCAGCCTGTGGGACCAGAGCCTGAAGCCCTGCGTGAAGCTGACCCCCCTGTGCGTGACCCTGAACTGCACCGACGACG
TGCGGAACGTGACCAACAACGCCACCAACACCAACAGCAGCTGGGGCGAGCCTATGGAAAAGGGCGAGATCAAGAACTGC
AGCTTCAACATCACCACCTCCATCCGGAACAAGGTGCAGAAGCAGTACGCCCTGTTCTACAAGCTGGACGTGGTGCCCAT
CGACAACGACAGCAACAACACCAACTACCGGCTGATCAGCTGCAACACCAGCGTGATCACCCAGGCCTGCCCCAAGGTGT
CCTTCGAGCCCATCCCCATCCACTACTGCGCCCCTGCCGGCTTCGCCATCCTGAAGTGCAACGACAAGAAGTTCAACGGC
ACCGGCCCCTGCACCAACGTGAGCACCGTGCAGTGCACCCACGGCATCCGGCCCGTGGTGTCCACCCAGCTGCTGCTGAA
CGGCAGCCTGGCCGAGGAAGAGGTGGTGATCAGAAGCGAGAATTTCACCAACAATGCCAAGACCATCATGGTGCAGCTGA
ACGTGAGCGTGGAGATCAACTGCACCCGGCCCAACAACAACACCCGGAAGAGCATCCACATCGGCCCTGGCAGGGCCTTC
TACACAGCCGGCGACATCATCGGCGACATCCGGCAGGCCCACTGCAACATCAGCCGGGCCAACTGGAACAACACCCTGCG
GCAGATCGTGGAGAAGCTGGGCAAGCAGTTCGGCAACAACAAGACCATCGTGTTCAACCACAGCAGCGGCGGAGACCCCG
AGATCGTGATGCACAGCTTCAACTGTGGCGGCGAGTTCTTCTACTGCAACAGCACCAAGCTGTTCAACAGCACCTGGACC
TGGAACAACTCCACCTGGAATAACACCAAGCGGAGCAACGACACCGAAGAGCACATCACCCTGCCCTGCCGGATCAAGCA
GATTATCAATATGTGGCAGGAGGTCGGCAAGGCCATGTACGCCCCTCCCATCCGGGGCCAGATCCGGTGCAGCAGCAACA
TCACCGGCCTGCTGCTGACCCGGGACGGCGGCAACGATACCAGCGGCACCGAGATCTTCCGGCCTGGCGGCGGAGATATG
CGGGACAACTGGCGGAGCGAGCTGTACAAGTACAAGGTGGTGAAGATCGAGCCCCTGGGCGTGGCTCCCACCAAGGCCAA
GCGGCGGGTGGTGCAGAGCGAGAAGAGCGCCGTGGGCATCGGCGCCGTGTTTCTGGGCTTCCTGGGAGCCGCCGGAAGCA
CCATGGGAGCCGCCAGCATGACCCTGACCGTGCAGGCCCGGCTGCTGCTGTCCGGCATCGTGCAGCAGCAGAACAACCTG
CTCCGGGCCATCGAGGCCCAGCAGCACCTGCTGCAGCTGACCGTGTGGGGCATCAAGCAGCTGCAGGCCAGGGTGCTGGC
CGTGGAGAGATACCTGAAGGATCAGCAGCTCCTGGGGATCTGGGGCTGCAGCGGCAAGCTGATCTGCACCACCACCGTGC
CCTGGAACGCCAGCTGGTCCAACAAGAGCCTGGACAAGATCTGGAACAATATGACCTGGATGGAATGGGAGCGCGAGATC
AACAATTACACCAGCCTGATCTACACCCTGATCGAGGAAAGCCAGAACCAGCAGGAAAAGAACGAGCAGGAACTGCTGGA
ACTGGACAAGTGGGCCAGCCTGTGGAACTGGTTCGACATCAGCAACTGGCTGTGG
nucleotide sequence encoding mos2S.Env (SEQ ID NO: 10)
ATGAGAGTGCGGGGCATGCTGAGAAACTGGCAGCAGTGGTGGATCTGGTCCAGCCTGGGCTTCTGGATGCTGATGATCTACAGCG
TGATGGGCAACCTGTGGGTCACCGTGTACTACGGCGTGCCCGTGTGGAAGGACGCCAAGACCACCCTGTTTTGCGCCTCCGATGC
CAAGGCCTACGAGAAAGAGGTGCACAACGTCTGGGCCACCCACGCCTGTGTGCCCACCGACCCCAATCCCCAGGAAATCGTCCTG
GGCAACGTGACCGAGAACTTCAACATGTGGAAGAACGACATGGTCGATCAGATGCACGAGGACATCATCTCCCTGTGGGACGCCT
CCCTGGAACCCTGCGTGAAGCTGACCCCTCTGTGCGTGACCCTGAACTGCCGGAACGTGCGCAACGTGTCCAGCAACGGCACCTA
CAACATCATCCACAACGAGACATACAAAGAGATGAAGAACTGCAGCTTCAACGCTACCACCGTGGTCGAGGACCGGAAGCAGAAG
GTGCACGCCCTGTTCTACCGGCTGGACATCGTGCCCCTGGACGAGAACAACAGCAGCGAGAAGTCCTCCGAGAACAGCTCCGAGT
ACTACAGACTGATCAACTGCAACACCAGCGCCATCACCCAGGCCTGCCCCAAGGTGTCCTTCGACCCTATCCCCATCCACTACTG
CGCCCCTGCCGGCTACGCCATCCTGAAGTGCAACAACAAGACCTTCAATGGCACCGGCCCCTGCAACAATGTGTCCACCGTGCAG
TGCACCCACGGCATCAAGCCCGTGGTGTCTACCCAGCTGCTGCTGAACGGCAGCCTGGCCGAGGAAGAGATCATTATCAGAAGCG
AGAACCTGACCAACAACGCCAAAACCATCATCGTCCACCTGAACGAAACCGTGAACATCACCTGTACCCGGCCTAACAACAACAC
CCGGAAGTCCATCCGGATCGGCCCTGGCCAGACCTTTTACGCCACCGGCGATATTATCGGCGACATCCGGCAGGCCCACTGCAAT
CTGAGCCGGGACGGCTGGAACAAGACACTGCAGGGCGTCAAGAAGAAGCTGGCCGAACACTTCCCTAACAAGACTATCAAGTTCG
CCCCTCACTCTGGCGGCGACCTGGAAATCACCACCCACACCTTCAACTGTCGGGGCGAGTTCTTCTACTGCAATACCTCCAACCT
GTTCAACGAGAGCAACATCGAGCGGAACGACAGCATCATCACACTGCCTTGCCGGATCAAGCAGATTATCAATATGTGGCAGGAA
GTGGGCAGAGCCATCTACGCCCCTCCAATCGCCGGCAACATCACATGCCGGTCCAATATCACCGGCCTGCTGCTCACCAGAGATG
GCGGCTCCAACAATGGCGTGCCAAACGACACCGAGACATTCAGACCCGGCGGAGGCGACATGCGGAACAATTGGCGGAGCGAGCT
GTACAAGTACAAGGTGGTGGAAGTGAAGCCCCTGGGCGTGGCCCCTACCGAGGCCAAGAGAAGAGTGGTCGAACGCGAGAAGCGG
GCCGTGGGAATCGGAGCCGTGTTTCTGGGAATCCTGGGAGCCGCTGGCTCTACCATGGGCGCTGCCTCTATCACCCTGACAGTGC
AGGCCAGACAGCTGCTCAGCGGCATCGTGCAGCAGCAGAGCAACCTGCTGAGAGCCATTGAGGCCCAGCAGCACATGCTGCAGCT
GACCGTGTGGGGCATTAAGCAGCTCCAGACACGGGTGCTGGCCATCGAGAGATACCTGCAGGATCAGCAGCTCCTGGGCCTGTGG
GGCTGTAGCGGCAAGCTGATCTGTACCACCGCCGTGCCCTGGAATACCTCTTGGAGCAACAAGAGCCAGACCGACATCTGGGACA
ACATGACCTGGATGCAGTGGGACAAAGAAATCGGCAACTATACCGGCGAGATCTATAGACTGCTGGAAGAGTCCCAGAACCAGCA
GGAAAAGAACGAGAAGGACCTGCTGGCCCTGGATTCTTGGAACAATCTGTGGAACTGGTTCAGCATCTCCAAGTGGCTGTGGTAC
ATCAAGATCTTCATCATGATCGTGGGCGGCCTGATCGGCCTGCGGATCATCTTTGCCGTGCTGAGCATCGTGAACCGCGTGCGGC
AGGGCTAC
mos1.GagPol polypeptide (SEQ ID NO: 11)
MGARASVLSGGELDRWEKIRLRPGGKKKYRLKHIVWASRELERFAVNPGLLETSEGCRQILGQLQPSLQTGSEELRSLYNTVATL
YCVHQRIEIKDTKEALEKIEEEQNKSKKKAQQAAADTGNSSQVSQNYPIVQNIQGQMVHQAISPRTLNAWVKVVEEKAFSPEVIP
MFSALSEGATPQDLNTMLNTVGGHQAAMQMLKETINEEAAEWDRVHPVHAGPIAPGQMREPRGSDIAGTTSTLQEQIGWMTNNPP
IPVGEIYKRWIILGLNKIVRMYSPVSILDIRQGPKEPFRDYVDRFYKTLRAEQASQDVKNWMTETLLVQNANPDCKTILKALGPA
ATLEEMMTACQGVGGPGHKARVLAEAMSQVTNSATIMMQRGNFRNQRKTVKCFNCGKEGHIAKNCRAPRKKGCWKCGKEGHQMKD
CTERQANFLGKIWPSNKGRPGNFLQNRPEPTAPPEESFRFGEETTTPSQKQEPIDKEMYPLASLKSLFGNDPSSQMAPISPIETV
PVKLKPGMDGPRVKQWPLTEEKIKALTAICEEMEKEGKITKIGPENPYNTPVFAIKKKDSTKWRKLVDFRELNKRTQDFWEVQLG
IPHPAGLKKKKSVTVLAVGDAYFSVPLDEGFRKYTAFTIPSTNNETPGIRYQYNVLPQGWKGSPAIFQCSMTRILEPFRAKNPEI
VIYQYMAALYVGSDLEIGQHRAKIEELREHLLKWGFTTPDKKHQKEPPFLWMGYELHPDKWTVQPIQLPEKDSWTVNDIQKLVGK
LNWASQIYPGIKVRQLCKLLRGAKALTDIVPLTEEAELELAENREILKEPVHGVYYDPSKDLIAEIQKQGHDQWTYQIYQEPFKN
LKTGKYAKMRTAHTNDVKQLTEAVQKIAMESIVIWGKTPKFRLPIQKETWETWWTDYWQATWIPEWEFVNTPPLVKLWYQLEKDP
IAGVETFYVAGAANRETKLGKAGYVTDRGRQKIVSLTETTNQKTALQAIYLALQDSGSEVNIVTASQYALGIIQAQPDKSESELV
NQIIEQLIKKERVYLSWVPAHKGIGGNEQVDKLVSSGIRKVLFLDGIDKAQEEHEKYHSNWRAMASDFNLPPVVAKEIVASCDQC
QLKGEAMHGQVDCSPGIWQLACTHLEGKIILVAVHVASGYIEAEVIPAETGQETAYFILKLAGRWPVKVIHTANGSNFTSAAVKA
ACWWAGIQQEFGIPYNPQSQGVVASMNKELKKIIGQVRDQAEHLKTAVQMAVFIHNFKRKGGIGGYSAGERIIDIIATDIQTKEL
QKQIIKIQNFRVYYRDSRDPIWKGPAKLLWKGEGAVVIQDNSDIKVVPRRKVKIIKDYGKQMAGADCVAGRQDED
mos2.GagPol polypeptide (SEQ ID NO: 12)
MGARASILRGGKLDKWEKIRLRPGGKKHYMLKHLVWASRELERFALNPGLLETSEGCKQIIKQLQPALQTGTEELRSLFNTVATL
YCVHAEIEVRDTKEALDKIEEEQNKSQQKTQQAKEADGKVSQNYPIVQNLQGQMVHQPISPRTLNAWVKVIEEKAFSPEVIPMFT
ALSEGATPQDLNTMLNTVGGHQAAMQMLKDTINEEAAEWDRLHPVHAGPVAPGQMREPRGSDIAGTTSNLQEQLAWMTSNPPIPV
GDIYKRWIILGLNKIVRMYSPTSILDIKQGPKEPFRDYVDRFEKTLRAEQATQDVKNWMTDTLLVQNANPDCKTILRALGPGATL
EEMMTACQGVGGPSHKARVLAEAMSQTNSTILMQRSNFKGSKRIVKCFNCGKEGHIARNCRAPRKKGCWKCGKEGHQMKDCTERQ
ANFLGKIWPSHKGRPGNFLQSRPEPTAPPAESFRFEETTPAPKQEPKDREPLTSLRSLFGSDPLSQMAPISPIETVPVKLKPGMD
GPKVKQWPLTEEKIKALVEICTEMEKEGKISKIGPENPYNTPIFATKKKDSTKWRKLVDFRELNKRTQDFWEVQLGIPHPAGLKK
KKSVTVLAVGDAYFSVPLDEDFRKYTAFTIPSINNETPGIRYQYNVLPQGWKGSPAIFQSSMTKILEPFRKQNPDIVIYQYMAAL
YVGSDLEIGQHRTKIEELRQHLLRWGFTTPDKKHQKEPPFLWMGYELHPDKWTVQPIVLPEKDSWTVNDIQKLVGKLNWASQIYA
GIKVKQLCKLLRGTKALTEVVPLTEEAELELAENREILKEPVHGVYYDPSKDLIAEIQKQGQGQWTYQIYQEPFKNLKTGKYARM
RGAHTNDVKQLTEAVQKIATESIVIWGKTPKFKLPIQKETWEAWWTEYWQATWIPEWEFVNTPPLVKLWYQLEKEPIVGAETFYV
AGAANRETKLGKAGYVTDRGRQKVVSLTDTTNQKTALQAIHLALQDSGLEVNIVTASQYALGIIQAQPDKSESELVSQIIEQLIK
KEKVYLAWVPAHKGIGGNEQVDKLVSRGIRKVLELDGIDKAQEEHEKYHSNWRAMASEFNLPPIVAKEIVASCDKCQLKGEATHG
QVDCSPGIWQLACTHLEGKVILVAVHVASGYIEAEVIPAETGQETAYFLLKLAGRWPVKTIHTANGSNFTSATVKAACWWAGIKQ
EFGIPYNPQSQGVVASINKELKKIIGQVRDQAEHLKTAVQMAVFIHNFKRKGGIGEYSAGERIVDIIASDIQTKELQKQITKIQN
FRVYYRDSRDPLWKGPAKLLWKGEGAVVIQDNSDIKVVPRRKAKIIRDYGKQMAGDDCVASRQDED

TABLE 2
Env PTE sub pools

| Sub | | | | SEQ ID |
|---|---|---|---|---|
| Pool | Sequence | Start | End | NO: |
| 1 | KEIRKNYQHLWRWGT | 1 | 15 | 13 |
| 1 | MNWPNLWKWGTLILG | 9 | 23 | 14 |
| 1 | YQHLWRWGTMLLGML | 11 | 25 | 15 |
| 1 | PQWWIWGILGFWMLM | 12 | 26 | 16 |
| 1 | WKWGTLILGLVIICS | 15 | 29 | 17 |
| 1 | WGTMLLGMLMICSAA | 17 | 31 | 18 |
| 1 | GILGFWMLMICNVVG | 18 | 32 | 19 |
| 1 | ILGLVIICSASDNLW | 21 | 35 | 20 |
| 1 | LGMLMICSATEKLWV | 22 | 36 | 21 |
| 1 | GMLMICSAAEQLWVT | 23 | 37 | 22 |
| 1 | ICNVVGNLWVTVYYG | 27 | 41 | 23 |
| 1 | ICSAAENLWVTVYYG | 27 | 41 | 24 |
| 1 | ICSASDNLWVTVYYG | 27 | 41 | 25 |
| 1 | CSATEKLWVTVYYGV | 28 | 42 | 26 |
| 1 | CSAAEQLWVTVYYGV | 28 | 42 | 27 |
| 1 | NLWVTVYYGVPVWKE | 33 | 47 | 28 |
| 1 | TVYYGVPVWRDAETT | 37 | 51 | 29 |
| 1 | VYYGVPVWKDAETTL | 38 | 52 | 30 |
| 1 | VYYGVPVWREAKTTL | 38 | 52 | 31 |
| 1 | YYGVPVWKEATTTLF | 39 | 53 | 32 |
| 1 | YGVPVWKEAKTTLLC | 40 | 54 | 33 |
| 2 | PVWRDAETTLFCASE | 43 | 57 | 34 |
| 2 | VWKEAKTTLFCASDA | 44 | 58 | 35 |
| 2 | VWKDAETTLFCASDA | 44 | 58 | 36 |
| 2 | VWREAKTTLFCASNA | 44 | 58 | 37 |
| 2 | WKEATTTLFCASDAR | 45 | 59 | 38 |
| 2 | DADTTLFCASDAKSY | 47 | 61 | 39 |
| 2 | ATTTLFCASDAKAYD | 48 | 62 | 40 |
| 2 | LLCASDAKAYEREVH | 52 | 66 | 41 |
| 2 | FCASDAKAYEKEVHN | 53 | 67 | 42 |
| 2 | CASDAKAYDTEVHNV | 54 | 68 | 43 |
| 2 | ASDARAYDTEVHNVW | 55 | 69 | 44 |
| 2 | AKAYEKEVHNVWATH | 58 | 72 | 45 |
| 2 | AKAYEREVHNVWATH | 58 | 72 | 46 |
| 2 | AYETEKHNVWATHAC | 60 | 74 | 47 |
| 2 | AYDTEAHNVWATHAC | 60 | 74 | 48 |
| 2 | YDTEVHNVWATHACV | 61 | 75 | 49 |
| 2 | TEAHNIWATHACVPT | 63 | 77 | 50 |
| 2 | NVWATHACVPTDPNP | 67 | 81 | 51 |
| 2 | ACVPTDPNPQEIHLE | 73 | 87 | 52 |
| 2 | CIPTDPNPQEIVLEN | 74 | 88 | 53 |
| 2 | VPTDPNPQEVVLGNV | 75 | 89 | 54 |
| 2 | VPTDPNPQEMVLENV | 75 | 89 | 55 |
| 2 | PNPQEIHLENVTEEF | 79 | 93 | 56 |
| 2 | NPQEIVLENVTENFN | 80 | 94 | 57 |
| 3 | PQEINLENVTEEFNM | 81 | 95 | 58 |
| 3 | VVLGNVTENFNMWEN | 84 | 98 | 59 |
| 3 | LENVTENFNMWKNNM | 86 | 100 | 60 |
| 3 | ENVTEEFNMWKNNMV | 87 | 101 | 61 |
| 3 | TENFNMWKNDMVDQM | 90 | 104 | 62 |
| 3 | NFNMWKNNMVEQMHE | 92 | 106 | 63 |
| 3 | NFDMWKNNMVEQMHT | 92 | 106 | 64 |
| 3 | MKNNMVEQMHEDIIS | 96 | 110 | 65 |
| 3 | WKNDMVDQMHEDVIS | 96 | 110 | 66 |
| 3 | KNDMVDQMHEDIISL | 97 | 111 | 67 |
| 3 | NNMVEQMHTDIISLW | 98 | 112 | 68 |
| 3 | NDMVNQMHEDVISLW | 98 | 112 | 69 |
| 3 | EQMHEDIISLWDQSL | 102 | 116 | 70 |
| 3 | HMHEDIISLWDESLK | 103 | 117 | 71 |
| 3 | MHTDIISLWDQSLKP | 104 | 118 | 72 |
| 3 | MHEDVISLWDQSLKP | 104 | 118 | 73 |
| 3 | ISLWDQSLKPCVKLT | 109 | 123 | 74 |
| 3 | ISLWDESLKPCVKLT | 109 | 123 | 75 |
| 3 | SLKPCVKLTPLCVTL | 115 | 129 | 76 |
| 3 | CVQLTPLCVTLNCSN | 119 | 133 | 77 |
| 4 | KLTPLCVTLNCTDVK | 121 | 135 | 78 |
| 4 | KLTPLCVTLDCTNAT | 121 | 135 | 79 |
| 4 | PLCVTLNCTNANITS | 124 | 138 | 80 |
| 4 | LCVTLNCTDLRNTTN | 125 | 139 | 81 |
| 4 | MEKGEIKNCSFNVTT | 149 | 163 | 82 |
| 4 | KGEIKNCSFNITTSI | 151 | 165 | 83 |
| 4 | GEIKNCSFNMTTEVR | 152 | 166 | 84 |
| 4 | EMKNCSFNITTELRD | 153 | 167 | 85 |
| 4 | EIKNCSFNTTTEIRD | 153 | 167 | 86 |
| 4 | IKNCSFNMTTELRDK | 154 | 168 | 87 |
| 4 | CSFNITTSIRDKVQK | 157 | 171 | 88 |
| 4 | NMTTELRDKKQKVYS | 160 | 174 | 89 |
| 4 | TSIRDKVQKEYALFY | 163 | 177 | 90 |
| 5 | KEYALFYKLDVVPID | 171 | 185 | 91 |
| 5 | KEYALFYRLDIVPLN | 171 | 185 | 92 |
| 5 | KVYALFYKLDIVPLN | 171 | 185 | 93 |
| 5 | KVYSLFYRLDVVPID | 171 | 185 | 94 |
| 5 | YSLFYKLDVVPIDND | 173 | 187 | 95 |
| 5 | SEYRLINCNTSTITQ | 189 | 203 | 96 |
| 5 | TSYRLINCNTSVITQ | 189 | 203 | 97 |
| 5 | TSYRLISCNTSVITQ | 189 | 203 | 98 |
| 5 | SQYRLINCNTSAVTQ | 189 | 203 | 99 |
| 5 | YRLINCNTSAITQAC | 191 | 205 | 100 |
| 5 | ILINCNTSVIKQACP | 192 | 206 | 101 |
| 5 | SCNTSVITQACPKVS | 195 | 209 | 102 |
| 5 | NCNTSTITQACPKVS | 195 | 209 | 103 |
| 5 | NTSAITQACPKVTFE | 197 | 211 | 104 |
| 5 | NVSTIKQACPKVSFE | 197 | 211 | 105 |
| 5 | TSVIKQACPKVTFEP | 198 | 212 | 106 |
| 5 | SAVTQACPKVTFDPI | 199 | 213 | 107 |
| 5 | AITQACPKVSFDPIP | 200 | 214 | 108 |
| 5 | ITQACPKVSFEPIPI | 201 | 215 | 109 |
| 5 | TQACPKVSWDPIPIH | 202 | 216 | 110 |
| 6 | ACPKVTFEPIPIHYC | 204 | 218 | 111 |
| 6 | CPKVTFDPIPIHYCT | 205 | 219 | 112 |
| 6 | PKVSFDPIPIHYCAP | 206 | 220 | 113 |
| 6 | KVSFEPIPIHYCTPA | 207 | 221 | 114 |
| 6 | SWDPIPIHYCAPAGY | 209 | 223 | 115 |
| 6 | FEPIPIHYCAPAGFA | 210 | 224 | 116 |
| 6 | DPIPIHYCTPAGFAI | 211 | 225 | 117 |
| 6 | IHYCAPAGYAILKCN | 215 | 229 | 118 |
| 6 | HYCAPAGFAILKCND | 216 | 230 | 119 |
| 6 | YCTPAGFAILKCKDK | 217 | 231 | 120 |
| 6 | CTPAGYAILKCNNKT | 218 | 232 | 121 |
| 6 | PAGFAILKCRDKKFN | 220 | 234 | 122 |
| 6 | AGFAILKCNNKTFSG | 221 | 235 | 123 |
| 6 | AGYAILKCNDKNFNG | 221 | 235 | 124 |
| 6 | GFAILKCNDKKFNGT | 222 | 236 | 125 |
| 6 | AILKCNNKTFNGTGP | 224 | 238 | 126 |
| 6 | CNDKKFNGTGLCKNV | 228 | 242 | 127 |
| 6 | NDKKFNGTGPCKNVS | 229 | 243 | 128 |
| 6 | NNKKFNGTGPCTNVS | 229 | 243 | 129 |
| 6 | NKTFNGTGPCNNVST | 230 | 244 | 130 |
| 6 | GTGPCKNVSTVQCTH | 235 | 249 | 131 |
| 6 | GTGPCTNVSTVQCTH | 235 | 249 | 132 |
| 6 | GTGPCHNVSTVQCTH | 235 | 249 | 133 |
| 6 | TGPCNNVSTVQCTHG | 236 | 250 | 134 |
| 6 | CKNVSSVQCTHGIKP | 239 | 253 | 135 |
| 6 | NVSTVQCTHGIKPVV | 241 | 255 | 136 |
| 6 | NISTVQCTHGIRPVV | 241 | 255 | 137 |
| 7 | CTHGIKPVVSTQLLL | 247 | 261 | 138 |
| 7 | CTHGIRPVVSTQLLL | 247 | 261 | 139 |
| 7 | PVVSTQLLLNGSLAE | 253 | 267 | 140 |
| 7 | LLLNGSLAEEEIIIR | 259 | 273 | 141 |
| 7 | LLLNGSLAEGEIIIR | 259 | 273 | 142 |
| 7 | LLLNGSLAEKEIIIR | 259 | 273 | 143 |
| 7 | LNGSLAEEEVVIRSE | 261 | 275 | 144 |
| 7 | LAEEEIIIRSENITN | 265 | 279 | 145 |
| 7 | LAEGEIIIRSQNISD | 265 | 279 | 146 |
| 7 | AEEEIIIRSENLTNN | 266 | 280 | 147 |
| 7 | AEGEIIIRSENLTDN | 266 | 280 | 148 |
| 7 | AEEEVMIRSENITNN | 266 | 280 | 149 |
| 7 | EIVIRSENLTNNVKT | 269 | 283 | 150 |
| 7 | IRSENITNNAKTIIV | 272 | 286 | 151 |
| 7 | IRSENLTNNAKIIIV | 272 | 286 | 152 |
| 7 | IRSENFTNNAKTIIV | 272 | 286 | 153 |
| 7 | SENLTDNVKTIIVHL | 274 | 288 | 154 |
| 7 | SENITNNAKNIIVQL | 274 | 288 | 155 |
| 7 | ENLTNNAKTIIVQLN | 275 | 289 | 156 |
| 7 | ENLTNNVKTIIVHLN | 275 | 289 | 157 |
| 7 | NFTDNAKTIIVQLKE | 276 | 290 | 158 |
| 7 | TNNAKTIIVHLNESV | 278 | 292 | 159 |
| 7 | AKTIIVQLNESVEIN | 281 | 295 | 160 |
| 7 | VKTIIVHLNESIEIV | 281 | 295 | 161 |
| 8 | IIVHLNESVEIVCTR | 284 | 298 | 162 |
| 8 | HLNQSVEIVCTRPNN | 287 | 301 | 163 |
| 8 | LNESVEINCTRPNNN | 288 | 302 | 164 |
| 8 | EIVCTRPNNNTRKGI | 293 | 307 | 165 |
| 8 | INCTRPNNNTRKSIR | 294 | 308 | 166 |
| 8 | INCTRPGNNTRKSIR | 294 | 308 | 167 |
| 8 | TCIRPNNNTRKSVRI | 295 | 309 | 168 |
| 8 | PNNNTRKSIHIGPGR | 299 | 313 | 169 |
| 8 | NNNTRKSIRIGPGQT | 300 | 314 | 170 |
| 8 | NNTRKSVRIGPGQTF | 301 | 315 | 171 |
| 8 | KSIHIGPGRAFYATG | 307 | 321 | 172 |
| 8 | SIRIGPGQTFYATGD | 308 | 322 | 173 |
| 8 | VRIGPGQTFYATGEI | 309 | 323 | 174 |
| 8 | RIGPGQAFYATGDII | 310 | 324 | 175 |
| 8 | GQTFYATGDIIGNIR | 313 | 327 | 176 |
| 8 | TGDIIGDIRKAHCNV | 319 | 333 | 177 |
| 8 | TGEIIGDIRQAHCNL | 319 | 333 | 178 |
| 8 | GDIIGDIRQAHCNIS | 320 | 334 | 179 |
| 8 | ITGDIRQAHCNVSRS | 322 | 336 | 180 |
| 9 | DIRQAHCNLSRAKWN | 325 | 339 | 181 |
| 9 | EQFGNKTIVFNQSSG | 352 | 366 | 182 |
| 9 | TIVFNQSSGGDPEIV | 358 | 372 | 183 |
| 9 | TIIFTNSSGGDLEIT | 358 | 372 | 184 |
| 9 | TIIFKPSSGGDLEIT | 358 | 372 | 185 |
| 9 | IIFNSSSGGDLEITT | 359 | 373 | 186 |
| 9 | FKPSSGGDPEITTHS | 361 | 375 | 187 |
| 9 | FAPSSGGDLEVTTHS | 361 | 375 | 188 |
| 9 | FQPPSGGDLEITMHH | 361 | 375 | 189 |
| 9 | SSGGDPEIVMHSFNC | 364 | 378 | 190 |
| 10 | SGGDLEITTHSFNCG | 365 | 379 | 191 |
| 10 | GDPEITTHSFNCRGE | 367 | 381 | 192 |
| 10 | GDIEITTHSFNCGGE | 367 | 381 | 193 |
| 10 | DLEIVMHSFNCGGEF | 368 | 382 | 194 |
| 10 | THSFNCGGEFFYCNT | 373 | 387 | 195 |
| 10 | THSFNCRGEFFYCNT | 373 | 387 | 196 |
| 10 | CGGEFFYCNSTQLFN | 378 | 392 | 197 |
| 10 | GGEFFYCNTSGLFNS | 379 | 393 | 198 |
| 10 | RGEFFYCNTSKLFNS | 379 | 393 | 199 |
| 10 | GGEFFYCNTTQLFNS | 379 | 393 | 200 |
| 10 | RGEFFYCNTTKLFNS | 379 | 393 | 201 |
| 10 | YCNSTQLFNSTWNST | 384 | 398 | 202 |
| 10 | CNTSGLFNSTWNDTG | 385 | 399 | 203 |
| 11 | NSTITIPCRIKQIIN | 411 | 425 | 204 |
| 11 | DTITLQCRIKQIINM | 412 | 426 | 205 |
| 11 | TITLPCRIKQIVNMW | 413 | 427 | 206 |
| 11 | TIILPCRIKQIINRW | 413 | 427 | 207 |
| 11 | ITLPCRIKQIINMWQ | 414 | 428 | 208 |
| 11 | CKIKQIINMWQGVGR | 418 | 432 | 209 |
| 11 | RIKQIVNMWQEVGRA | 419 | 433 | 210 |
| 11 | RIRQIINMWQEVGKA | 419 | 433 | 211 |
| 11 | IKQIINMWQEVGRAM | 420 | 434 | 212 |
| 11 | IKQIINMWQRVGQAM | 420 | 434 | 213 |
| 11 | INMWQGVGRAMYAPP | 424 | 438 | 214 |
| 11 | NMWQEVGKAMYAPPI | 425 | 439 | 215 |
| 11 | MWQEVGRAMYAPPIA | 426 | 440 | 216 |
| 11 | MWQRVGQAMYAPPIQ | 426 | 440 | 217 |
| 11 | RAGQAMYAPPIPGVI | 429 | 443 | 218 |
| 11 | GRAMYAPPIEGNITC | 431 | 445 | 219 |
| 11 | GKAMYAPPISGQIRC | 431 | 445 | 220 |
| 11 | GKAMYAPPIRGQIRC | 431 | 445 | 221 |
| 11 | GQAMYAPPIQGVIRC | 431 | 445 | 222 |
| 11 | RAMYAPPIAGNITCK | 432 | 446 | 223 |
| 11 | APPIEGNITCKSNIT | 436 | 450 | 224 |
| 11 | PPISGQIRCSSKITG | 437 | 451 | 225 |
| 11 | PIAGNITCKSNITGI | 438 | 452 | 226 |
| 11 | ISGQIRCSSNITGLL | 439 | 453 | 227 |
| 11 | GNITCKSNITGLLLT | 441 | 455 | 228 |
| 11 | GVIRCESNITGLLLT | 441 | 455 | 229 |
| 11 | GRIICKSNITGLLLV | 441 | 455 | 230 |
| 11 | CSSNITGLLLTRDGG | 445 | 459 | 231 |
| 11 | CTSNITGLLLVRDGG | 445 | 459 | 232 |
| 11 | SSNITGLILTRDGGN | 446 | 460 | 233 |
| 12 | GLLLTRDGGNNNNGS | 451 | 465 | 234 |
| 12 | STNETFRPGGGDMRD | 463 | 477 | 235 |
| 12 | NDTEIFRPGGGDMRN | 463 | 477 | 236 |
| 12 | NETFRPGGGNMKDNW | 465 | 479 | 237 |
| 12 | TEIFRPGGGNMRDNW | 465 | 479 | 238 |
| 12 | TETFRPGGGDMKDNW | 465 | 479 | 239 |
| 12 | RPGGGDMRDNWRSEL | 469 | 483 | 240 |
| 12 | GGGNMKDNWRSELYK | 471 | 485 | 241 |
| 12 | MRDNWRSELYKYKVV | 475 | 489 | 242 |
| 12 | SELYKYKVVKIEPLG | 481 | 495 | 243 |
| 12 | SELYKYKVVEIKPLG | 481 | 495 | 244 |
| 12 | SELYKYKVVRIEPLG | 481 | 495 | 245 |
| 12 | LYKYKVVEIEPLGVA | 483 | 497 | 246 |
| 12 | YKVVQIEPLGVAPTR | 486 | 500 | 247 |
| 13 | KVVKIEPLGVAPTKA | 487 | 501 | 248 |
| 13 | KVVEIKPLGVAPTKA | 487 | 501 | 249 |
| 13 | KVVEIKPLGIAPTKA | 487 | 501 | 250 |
| 13 | EPLGVAPTRAKRRVV | 492 | 506 | 251 |
| 13 | PLGVAPTKAKRRVVQ | 493 | 507 | 252 |
| 13 | IGLAPTKAKRRVVQR | 494 | 508 | 253 |
| 13 | GIAPTKAKRRVVERE | 495 | 509 | 254 |
| 13 | GIAPTRAKRRVVERE | 495 | 509 | 255 |
| 13 | GVAPTEAKRRVVERE | 495 | 509 | 256 |
| 13 | APTKARRRVVEREKR | 497 | 511 | 257 |
| 13 | KAKRRVVQREKRAVG | 500 | 514 | 258 |
| 13 | AKRRVVEREKRAVGI | 501 | 515 | 259 |
| 13 | RVVEREKRAIGLGAM | 504 | 518 | 260 |
| 13 | VQREKRAVGIGALFL | 506 | 520 | 261 |
| 13 | VEREKRAVGLGALFL | 506 | 520 | 262 |
| 13 | EREKRAVGIGAVFLG | 507 | 521 | 263 |
| 13 | EKEKRAIGLGAMFLG | 507 | 521 | 264 |
| 13 | REKRAVGLGAVFLGF | 508 | 522 | 265 |
| 13 | RAVGTIGAMFLGFLG | 510 | 524 | 266 |
| 13 | IGLGAMFLGFLGAAG | 513 | 527 | 267 |
| 13 | VGIGALFLGFLGAAG | 513 | 527 | 268 |
| 13 | VGIGAVFLGFLGVAG | 513 | 527 | 269 |
| 13 | GIGAVFLGFLGAAGS | 514 | 528 | 270 |
| 13 | GLGAVFLGFLGTAGS | 514 | 528 | 271 |
| 13 | IGAMIFGFLGAAGST | 515 | 529 | 272 |
| 13 | LGAVFIGFLGAAGST | 515 | 529 | 273 |
| 13 | LGFLGAAGSTMGAAS | 520 | 534 | 274 |
| 13 | AGSTMGAASITLTVQ | 526 | 540 | 275 |
| 13 | AGSTMGAASMTLTVQ | 526 | 540 | 276 |
| 13 | AGSTMGAASLTLTVQ | 526 | 540 | 277 |
| 14 | MGARSMTLTVQARQL | 530 | 544 | 278 |
| 14 | AASITLTVQARQLLS | 532 | 546 | 279 |
| 14 | AASMTLTVQARLLLS | 532 | 546 | 280 |
| 14 | AASLTLTVQARQLMS | 532 | 546 | 281 |
| 14 | TVQARQLLSGIVQQQ | 538 | 552 | 282 |
| 14 | TVQARLLLSGIVQQQ | 538 | 552 | 283 |
| 14 | LLSGIVQQQSNLLRA | 544 | 558 | 284 |
| 14 | LLSGIVQQQNNLLRA | 544 | 558 | 285 |
| 14 | VVQQQSNLLRAIEAQ | 548 | 562 | 286 |
| 14 | VQQQNNLLRAIEAQH | 549 | 563 | 287 |
| 14 | VQQQSNLLKAIEAQQ | 549 | 563 | 288 |
| 14 | NLLRAIEAQQHLLQL | 554 | 568 | 289 |
| 14 | NLLSGIVQQQSNLLK | 554 | 568 | 290 |
| 14 | LLKAIEAQQHLLKLT | 555 | 569 | 291 |
| 14 | LLMAIEAQQHLLQLT | 555 | 569 | 292 |
| 14 | LRAIEAQQHMLQLTA | 556 | 570 | 293 |
| 14 | EAQQHMLQLTVWGIK | 560 | 574 | 294 |
| 14 | AQQHLLQLTVWGIKQ | 561 | 575 | 295 |
| 14 | AQQHLLKLTVWGIKQ | 561 | 575 | 296 |
| 14 | AQQHLLRLTVWGIKQ | 561 | 575 | 297 |
| 14 | HMLKLTVWGIKQLQA | 564 | 578 | 298 |
| 14 | HMLRLTVWGIKQLQA | 564 | 578 | 299 |
| 14 | VQLTVWGIKQLQTRV | 566 | 580 | 300 |
| 15 | LTVWGIKQLQARVLA | 568 | 582 | 301 |
| 15 | WGIKQLQARILAVER | 571 | 585 | 302 |
| 15 | GIKQLQTRVLAIERY | 572 | 586 | 303 |
| 15 | KQLQARVLAVERYLK | 574 | 588 | 304 |
| 15 | KQLQARVLALERYLK | 574 | 588 | 305 |
| 15 | KQLQARVLAIERYLQ | 574 | 588 | 306 |
| 15 | TRVLAIERYLKDQQL | 578 | 592 | 307 |
| 15 | TRVLAVERYLRDQQL | 578 | 592 | 308 |
| 15 | VLAVERYLKDQQFLG | 580 | 594 | 309 |
| 15 | VERYLKDQQLLGIWG | 583 | 597 | 310 |
| 15 | VERYLRDQQLLGIWG | 583 | 597 | 311 |
| 15 | VEKYLKDQQLLGLWG | 583 | 597 | 312 |
| 15 | ILRNYQQWWIWGILG | 586 | 600 | 313 |
| 15 | DQQLLGIWGCSGKLI | 589 | 603 | 314 |
| 15 | DQQLLGLWGCSGKLI | 589 | 603 | 315 |
| 15 | LGIWGCSGKHICTTN | 593 | 607 | 316 |
| 15 | IWGCSGKLICTTNVP | 595 | 609 | 317 |
| 15 | LWGCSGKLICTTSVP | 595 | 609 | 318 |
| 15 | CSGKLICTTAVPWNS | 598 | 612 | 319 |
| 15 | CSGKLICTTTVPWNS | 598 | 612 | 320 |
| 15 | SGKIICTTAVPWNAS | 599 | 613 | 321 |
| 15 | KLICTTNVPWNSTWS | 601 | 615 | 322 |
| 15 | HICTTNVPWNASWSN | 602 | 616 | 323 |
| 15 | ICTTTVPWNASWSNR | 603 | 617 | 324 |
| 15 | CTTNVPWNSSWSNKS | 604 | 618 | 325 |
| 15 | CTTAVPWNSSWSNRS | 604 | 618 | 326 |
| 15 | CTTTVPWNSSWSNKT | 604 | 618 | 327 |
| 15 | TTAVPWNASWSNKSL | 605 | 619 | 328 |
| 15 | TTAVPWNTSWSNKSL | 605 | 619 | 329 |
| 16 | WNSSWSNKSQEEIWN | 610 | 624 | 330 |
| 16 | WNSSWSNKSLDKIWN | 610 | 624 | 331 |
| 16 | WNSSWSNKTYNDIWD | 610 | 624 | 332 |
| 16 | NASWSNKSLDDIWNN | 611 | 625 | 333 |
| 16 | NKSQSEIWDNMTWMQ | 616 | 630 | 334 |
| 16 | SQEEIWNNMTWMQWD | 618 | 632 | 335 |
| 16 | LNEIWDNMTWLQWDK | 619 | 633 | 336 |
| 16 | YNDIWDNMTWMQWER | 619 | 633 | 337 |
| 16 | EIWNNMTWMEWEKEI | 621 | 635 | 338 |
| 16 | DIWNNMTWMQWEKEI | 621 | 635 | 339 |
| 16 | IWDNMTWMQWDREIS | 622 | 636 | 340 |
| 16 | DNMTWMQWDKEISNY | 624 | 638 | 341 |
| 16 | NNMTWMEWEREINNY | 624 | 638 | 342 |
| 16 | NMTWLQWDKEISNYT | 625 | 639 | 343 |
| 16 | NMTWMEWEREIDNYT | 625 | 639 | 344 |
| 16 | NMTWMQWEREIDNYT | 625 | 639 | 345 |
| 16 | TWMQWDREINNYTNT | 627 | 641 | 346 |
| 16 | WMQWDREISNYTDTI | 628 | 642 | 347 |
| 16 | WEREIDNYTSLIYTL | 631 | 645 | 348 |
| 16 | REISNYTNTIYRLLE | 633 | 647 | 349 |
| 16 | EISNYTDTIYRLLEV | 634 | 648 | 350 |
| 16 | TGLIYTLIEESQNQQ | 639 | 653 | 351 |
| 16 | TNTIYRLLEDSQNQQ | 639 | 653 | 352 |
| 16 | TIYRLLEESQNQQEK | 641 | 655 | 353 |
| 16 | LIEESQNQQEKNEQD | 645 | 659 | 354 |
| 16 | LLEDSQNQQEKNEQE | 645 | 659 | 355 |
| 17 | ESQTQQEKNEQELLA | 648 | 662 | 356 |
| 17 | SQNQQEKNEKDLLAL | 649 | 663 | 357 |
| 17 | NQQEKNEQDLLALDK | 651 | 665 | 358 |
| 17 | NQQEKNEQELLELDK | 651 | 665 | 359 |
| 17 | NQQEQNEKDLLALDK | 651 | 665 | 360 |
| 17 | EKNEQELLALDKWAS | 654 | 668 | 361 |
| 17 | KNEKDLLALDSWNNL | 655 | 669 | 362 |
| 17 | NEKDLLALDSWKNLW | 656 | 670 | 363 |
| 17 | EQDLLALDKWASLWN | 657 | 671 | 364 |
| 17 | EQELLELDKWASLWS | 657 | 671 | 365 |
| 17 | DLLALDKWANLWNWF | 659 | 673 | 366 |
| 17 | LELDKWASLWNWFDI | 661 | 675 | 367 |
| 17 | ALDSWKNLWSWFDIT | 662 | 676 | 368 |
| 17 | ALDSWKNLWNWFDIT | 662 | 676 | 369 |
| 17 | LDTWASLWNWFSITN | 663 | 677 | 370 |
| 17 | TWASLWNWFDITNWL | 665 | 679 | 371 |
| 17 | KWASLWNWFSITKWL | 665 | 679 | 372 |
| 17 | SWKNLWNWFDISNWL | 665 | 679 | 373 |
| 17 | WANLWNWFDITKWLW | 666 | 680 | 374 |
| 17 | LWNWFSITNWLWYIK | 669 | 683 | 375 |

1. A vaccine combination for use in the treatment of HIV in a subject, comprising:
i) a first composition comprising a poxvirus vector comprising a polynucleotide that encodes at least one HIV envelope (Env) antigen;
ii) a second composition comprising a human adenovirus vector comprising a polynucleotide that encodes at least one HIV Env antigen,
wherein the first composition is for administration to the subject and the second composition is for administration to the subject less than six weeks after administration of the first composition.

2. The vaccine combination according to claim 1, wherein the time interval between administration of the first and second composition to the subject is 4 to 25 days.

3. The vaccine combination according to claim 1, wherein the use induces a broad T-cell immune response in the subject, wherein the broad T-cell immune response is characterized by a significant increase of positive peptide pools as compared to the response obtained in the same assay when the first administration to the subject is with the second composition and the second administration to the subject is with the first composition with the same interval between the first and second administration.

4. The vaccine combination according to claim 1, wherein the use induces a T-cell immune response in the subject, wherein the T-cell response is characterized by a significant increase of immune cells responding to the at least one HIV Env antigen in an interferon-gamma ELISpot assay as compared to the response obtained in the same assay when the first administration to the subject is with the second composition and the second administration to the subject is with the first composition with the same interval between the first and second administration.

5. A method for generating a broad T-cell immune response to one or more HIV Env antigens in a subject, the method comprising:
i) administering a first composition comprising a poxvirus vector comprising a polynucleotide that encodes at least one HIV Env antigen;
ii) administering a second composition less than 6 weeks after administration of the first composition, wherein the second composition comprises a human adenovirus vector comprising a polynucleotide that encodes at least one HIV Env antigen,
wherein the broad T-cell immune response is characterized by a significant increase of the number of positive peptide pools from the at least one HIV Env antigen in an interferon-gamma ELISpot assay as compared to the response obtained in the same assay when the first administration to the subject is with the second composition and the second administration to the subject is with the first composition with the same interval between the first and second administration.

6. The method according to claim 5, wherein the T-cell immune response is further characterized by a significant increase of immune cells responding to the at least one HIV Env antigen in an interferon-gamma ELISpot assay as compared to the response obtained in the same assay when the first administration to the subject is with the second composition and the second administration to the subject is with the first composition with the same interval between the first and second administration.

7. The method according to claim 5, wherein the interferon-gamma ELISpot assay is performed with 17 Env peptide pools, wherein a first pool comprises Env peptides having SEQ ID NOs: 13-33, a second pool comprises Env peptides having SEQ ID NOs: 34-57, a third pool comprises Env peptides having SEQ ID NOs: 58-77, a fourth pool comprises Env peptides having SEQ ID NOs: 78-90, a fifth pool comprises Env peptides having SEQ ID NOs: 91-110, a sixth pool comprises Env peptides having SEQ ID NOs: 111-137, a seventh pool comprises Env peptides having SEQ ID NOs: 138-161, an eighth pool comprises Env peptides having SEQ ID NOs: 162-180, a ninth pool comprises Env peptides having SEQ ID NOs: 181-190, a tenth pool comprises Env peptides having SEQ ID NOs: 191-203, an eleventh pool comprises Env peptides having SEQ ID NOs: 204-233, a twelfth pool comprises Env peptides having SEQ ID NOs: 234-247, a thirteenth pool comprises Env peptides having SEQ ID NOs: 248-277, a fourteenth pool comprises Env peptides having SEQ ID NOs: 278-300, a fifteenth pool comprises Env peptides having SEQ ID NOs: 301-329, a sixteenth pool comprises Env peptides having SEQ ID NOs: 330-355, and a seventeenth pool comprises Env peptides having SEQ ID NOs: 356-375

8. The method according to claim 5, wherein the time interval between administration of the first and second composition is 4 to 25 days.

9. The vaccine combination according to claim 1, wherein the at least one HIV Env antigen encoded in the first composition is substantially identical to the at least one HIV Env antigen encoded in the second composition.

10. A kit comprising:
i) a first composition comprising a poxvirus vector comprising a polynucleotide that encodes at least one HIV Env antigen;
ii) a second composition comprising a human adenovirus vector comprising a polynucleotide that encodes at least one HIV Env antigen; and
iii) a manual of instructions detailing that the second composition is to be administered after administration of the first composition to a subject and that the time interval between the administration of the first and the second composition to the subject is less than 6 weeks.

11. The kit according to claim 10, for use in treating HIV infection.

12. The vaccine combination according to claim 1, wherein the poxvirus vector is MVA.

13. The vaccine combination according to claim 1, wherein the human adenovirus vector is Ad26.

14. The vaccine combination according to claim 1, wherein the HIV Env antigen comprises the amino acid sequence of SEQ ID NO: 5 and/or SEQ ID NO: 6.

15. (canceled)

16. (canceled)

17. The method according to claim 5, wherein the time interval between administration of the first and second composition is 10 to 18 days.

18. The method according to claim 5, wherein the at least one HIV Env antigen encoded in the first composition is substantially identical to the at least one HIV Env antigen encoded in the second composition.

19. The method according to claim 5, wherein the at least one HIV Env antigen encoded in the first composition is identical to the at least one HIV Env antigen encoded in the second composition.

20. The method according to claim 5, wherein the poxvirus vector is MVA.

21. The method according to claim 5, wherein the human adenovirus vector is Ad26.

22. The method according to claim 5, wherein the HIV Env antigen comprises the amino acid sequence of SEQ ID NO: 5 and/or SEQ ID NO: 6.


SEQUENCE LISTING (375 sequences)

SEQ ID NO: 1 - protein, 500 aa, Artificial Sequence
  mos1.Gag polypeptide
MGARASVLSGGELDRWEKIRLRPGGKKKYRLKHIVWASRELERFAVNPGLLETSEGCRQILGQLQPSLQTGSEELRSLYNTVATLYCVHQRIEIKDTKEALEKIEEEQNKSKKKAQQAAADTGNSSQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPVSILDIRQGPKEPFRDYVDRFYKTLRAEQASQDVKNWMTETLLVQNANPDCKTILKALGPAATLEEMMTACQGVGGPGHKARVLAEAMSQVTNSATIMMQRGNFRNQRKTVKCFNCGKEGHIAKNCRAPRKKGCWKCGKEGHQMKDCTERQANFLGKIWPSNKGRPGNFLQNRPEPTAPPEESFRFGEETTTPSQKQEPIDKEMYPLASLKSLFGNDPSSQ

SEQ ID NO: 2 - protein, 491 aa, Artificial Sequence
  mos2.Gag polypeptide
MGARASILRGGKLDKWEKIRLRPGGKKHYMLKHLVWASRELERFALNPGLLETSEGCKQIIKQLQPALQTGTEELRSLFNTVATLYCVHAEIEVRDTKEALDKIEEEQNKSQQKTQQAKEADGKVSQNYPIVQNLQGQMVHQPISPRTLNAWVKVIEEKAFSPEVIPMFTALSEGATPQDLNTMLNTVGGHQAAMQMLKDTINEEAAEWDRLHPVHAGPVAPGQMREPRGSDIAGTTSNLQEQIAWMTSNPPIPVGDIYKRWIILGLNKIVRMYSPTSILDIKQGPKEPFRDYVDRFFKTLRAEQATQDVKNWMTDTLLVQNANPDCKTILRALGPGATLEEMMTACQGVGGPSHKARVLAEAMSQTNSTILMQRSNFKGSKRIVKCFNCGKEGHIARNCRAPRKKGCWKCGKEGHQMKDCTERQANFLGKIWPSHKGRPGNFLQSRPEPTAPPAESFRFEETTPAPKQEPKDREPLTSLRSLFGSDPLSQ

SEQ ID NO: 3 - protein, 850 aa, Artificial Sequence
  mos1.Pol polypeptide
MAPISPIETVPVKLKPGMDGPRVKQWPLTEEKIKALTAICEEMEKEGKITKIGPENPYNTPVFAIKKKDSTKWRKLVDFRELNKRTQDFWEVQLGIPHPAGLKKKKSVTVLAVGDAYFSVPLDEGFRKYTAFTIPSTNNETPGIRYQYNVLPQGWKGSPAIFQCSMTRILEPFRAKNPEIVIYQYMAALYVGSDLEIGQHRAKIEELREHLLKWGFTTPDKKHQKEPPFLWMGYELHPDKWTVQPIQLPEKDSWTVNDIQKLVGKLNWASQIYPGIKVRQLCKLLRGAKALTDIVPLTEEAELELAENREILKEPVHGVYYDPSKDLIAEIQKQGHDQWTYQIYQEPFKNLKTGKYAKMRTAHTNDVKQLTEAVQKIAMESIVIWGKTPKFRLPIQKETWETWWTDYWQATWIPEWEFVNTPPLVKLWYQLEKDPIAGVETFYVAGAANRETKLGKAGYVTDRGRQKIVSLTETTNQKTALQAIYLALQDSGSEVNIVTASQYALGIIQAQPDKSESELVNQIIEQLIKKERVYLSWVPAHKGIGGNEQVDKLVSSGIRKVLFLDGIDKAQEEHEKYHSNWRAMASDFNLPPVVAKEIVASCDQCQLKGEAMHGQVDCSPGIWQLACTHLEGKIILVAVHVASGYIEAEVIPAETGQETAYFILKLAGRWPVKVIHTANGSNFTSAAVKAACWWAGIQQEFGIPYNPQSQGVVASMNKELKKIIGQVRDQAEHLKTAVQMAVFIHNFKRKGGIGGYSAGERIIDIIATDIQTKELQKQIIKIQNFRVYYRDSRDPIWKGPAKLLWKGEGAVVIQDNSDIKVVPRRKVKIIKDYGKQMAGADCVAGRQDED

SEQ ID NO: 4 - protein, 850 aa, Artificial Sequence
  mos2.Pol polypeptide
MAPISPIETVPVKLKPGMDGPKVKQWPLTEEKIKALVEICTEMEKEGKISKIGPENPYNTPIFAIKKKDSTKWRKLVDFRELNKRTQDFWEVQLGIPHPAGLKKKKSVTVLAVGDAYFSVPLDEDFRKYTAFTIPSINNETPGIRYQYNVLPQGWKGSPAIFQSSMTKILEPFRKQNPDIVIYQYMAALYVGSDLEIGQHRTKIEELRQHLLRWGFTTPDKKHQKEPPFLWMGYELHPDKWTVQPIVLPEKDSWTVNDIQKLVGKLNWASQIYAGIKVKQLCKLLRGTKALTEVVPLTEEAELELAENREILKEPVHGVYYDPSKDLIAEIQKQGQGQWTYQIYQEPFKNLKTGKYARMRGAHTNDVKQLTEAVQKIATESIVIWGKTPKFKLPIQKETWEAWWTEYWQATWIPEWEFVNTPPLVKLWYQLEKEPIVGAETFYVAGAANRETKLGKAGYVTDRGRQKVVSLTDTTNQKTALQAIHLALQDSGLEVNIVTASQYALGIIQAQPDKSESELVSQIIEQLIKKEKVYLAWVPAHKGIGGNEQVDKLVSRGIRKVLFLDGIDKAQEEHEKYHSNWRAMASEFNLPPIVAKEIVASCDKCQLKGEAIHGQVDCSPGIWQLACTHLEGKVILVAVHVASGYIEAEVIPAETGQETAYFLLKLAGRWPVKTIHTANGSNFTSATVKAACWWAGIKQEFGIPYNPQSQGVVASINKELKKIIGQVRDQAEHLKTAVQMAVFIHNFKRKGGIGEYSAGERIVDIIASDIQTKELQKQITKIQNFRVYYRDSRDPLWKGPAKLLWKGEGAVVIQDNSDIKVVPRRKAKIIRDYGKQMAGDDCVASRQDED

SEQ ID NO: 5 - protein, 685 aa, Artificial Sequence
  mos1.Env polypeptide
MRVTGIRKNYQHLWRWGTMLLGILMICSAAGKLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDDVRNVTNNATNTNSSWGEPMEKGEIKNCSFNITTSIRNKVQKQYALFYKLDVVPIDNDSNNTNYRLISCNTSVITQACPKVSFEPIPIHYCAPAGFAILKCNDKKFNGTGPCTNVSTVQCTHGIRPVVSTQLLLNGSLAEEEVVIRSENFTNNAKTIMVQLNVSVEINCTRPNNNTRKSIHIGPGRAFYTAGDIIGDIRQAHCNISRANWNNTLRQIVEKLGKQFGNNKTIVFNHSSGGDPEIVMHSFNCGGEFFYCNSTKLFNSTWTWNNSTWNNTKRSNDTEEHITLPCRIKQIINMWQEVGKAMYAPPIRGQIRCSSNITGLLLTRDGGNDTSGTEIFRPGGGDMRDNWRSELYKYKVVKIEPLGVAPTKAKRRVVQSEKSAVGIGAVFLGFLGAAGSTMGAASMTLTVQARLLLSGIVQQQNNLLRAIEAQQHLLQLTVWGIKQLQARVLAVERYLKDQQLLGIWGCSGKLICTTTVPWNASWSNKSLDKIWNNMTWMEWEREINNYTSLIYTLIEESQNQQEKNEQELLELDKWASLWNWFDISNWLW

SEQ ID NO: 6 - protein, 711 aa, Artificial Sequence
  mos2S.Env polypeptide
MRVRGMLRNWQQWWIWSSLGFWMLMIYSVMGNLWVTVYYGVPVWKDAKTTLFCASDAKAYEKEVHNVWATHACVPTDPNPQEIVLGNVTENFNMWKNDMVDQMHEDIISLWDASLEPCVKLTPLCVTLNCRNVRNVSSNGTYNIIHNETYKEMKNCSFNATTVVEDRKQKVHALFYRLDIVPLDENNSSEKSSENSSEYYRLINCNTSAITQACPKVSFDPIPIHYCAPAGYAILKCNNKTFNGTGPCNNVSTVQCTHGIKPVVSTQLLLNGSLAEEEIIIRSENLTNNAKTIIVHLNETVNITCTRPNNNTRKSIRIGPGQTFYATGDIIGDIRQAHCNLSRDGWNKTLQGVKKKLAEHFPNKTIKFAPHSGGDLEITTHTFNCRGEFFYCNTSNLFNESNIERNDSIITLPCRIKQIINMWQEVGRAIYAPPIAGNITCRSNITGLLLTRDGGSNNGVPNDTETFRPGGGDMRNNWRSELYKYKVVEVKPLGVAPTEAKRRVVEREKRAVGIGAVFLGILGAAGSTMGAASITLTVQARQLLSGIVQQQSNLLRAIEAQQHMLQLTVWGIKQLQTRVLAIERYLQDQQLLGLWGCSGKLICTTAVPWNTSWSNKSQTDIWDNMTWMQWDKEIGNYTGEIYRLLEESQNQQEKNEKDLLALDSWNNLWNWFSISKWLWYIKIFIMIVGGLIGLRIIFAVLSIVNRVRQGY

SEQ ID NO: 7 - DNA, 4050 bp, Artificial Sequence
  mos1.GagPol  nt sequence
atgggagccagagccagcgtgctgtccggaggggagctggaccgctgggagaagatcaggctgaggcctggagggaagaagaagtacaggctgaagcacatcgtgtgggccagcagagagctggaacggtttgccgtgaaccctggcctgctggaaaccagcgagggctgtaggcagattctgggacagctgcagcccagcctgcagacaggcagcgaggaactgcggagcctgtacaacaccgtggccaccctgtactgcgtgcaccagcggatcgagatcaaggacaccaaagaagccctggaaaagatcgaggaagagcagaacaagagcaagaagaaagcccagcaggctgccgctgacacaggcaacagcagccaggtgtcccagaactaccccatcgtgcagaacatccagggacagatggtgcaccaggccatcagccctcggaccctgaacgcctgggtgaaggtggtggaggaaaaggccttcagccctgaggtgatccccatgttctctgccctgagcgagggagccacaccccaggacctgaacaccatgctgaacaccgtgggagggcaccaggctgccatgcagatgctgaaagagacaatcaacgaggaagctgccgagtgggacagggtccacccagtgcacgctggacctatcgctcctggccagatgagagagcccagaggcagcgatattgctggcaccacctccacactgcaggaacagatcggctggatgaccaacaaccctcccatccctgtgggagagatctacaagcggtggatcattctgggactgaacaagatcgtgcggatgtacagccctgtgagcatcctggacatcaggcagggacccaaagagcccttcagggactacgtggaccggttctacaagaccctgagagccgagcaggccagccaggacgtgaagaactggatgaccgagacactgctggtgcagaacgccaaccctgactgcaagaccatcctgaaagccctgggacctgctgccaccctggaagagatgatgacagcctgccagggagtgggaggacctggccacaaggccagggtgctggccgaggccatgagccaggtgaccaactctgccaccatcatgatgcagagaggcaacttccggaaccagagaaagaccgtgaagtgcttcaactgtggcaaagagggacacattgccaagaactgcagggctcccaggaagaaaggctgctggaagtgcggaaaagaaggccaccagatgaaggactgcaccgagaggcaggccaacttcctgggcaagatctggcctagcaacaagggcaggcctggcaacttcctgcagaacagacccgagcccaccgctcctcccgaggaaagcttccggtttggcgaggaaaccaccacccctagccagaagcaggaacccatcgacaaagagatgtaccctctggccagcctgaagagcctgttcggcaacgaccccagcagccagatggctcccatcagcccaatcgagacagtgcctgtgaagctgaagcctggcatggacggacccagggtgaagcagtggcctctgaccgaggaaaagatcaaagccctgacagccatctgcgaggaaatggaaaaagagggcaagatcaccaagatcggacccgagaacccctacaacacccctgtgttcgccatcaagaagaaagacagcaccaagtggaggaaactggtggacttcagagagctgaacaagcggacccaggacttctgggaggtgcagctgggcatccctcaccctgctggcctgaagaaaaagaaaagcgtgaccgtgctggctgtgggagatgcctacttcagcgtgcctctggacgagggcttccggaagtacacagccttcaccatccccagcaccaacaacgagacacctggcatcagataccagtacaacgtgctgcctcagggctggaaaggcagccctgccatcttccagtgcagcatgaccagaatcctggaacccttcagagccaagaaccctgagatcgtgatctaccagtatatggctgccctctacgtgggcagcgacctggaaatcggacagcacagagccaaaatcgaagaactccgcgagcacctgctgaagtggggattcaccacccctgacaagaagcaccagaaagagcctcccttcctgtggatgggctacgagctgcaccctgacaagtggaccgtgcagcccatccagctgccagagaaggactcctggaccgtgaacgacatccagaaactggtcggcaagctgaactgggccagccagatctaccctggcatcaaagtcagacagctgtgtaagctgctgaggggagccaaagcactgaccgacatcgtgcctctgacagaagaagccgagctggaactggccgagaacagagagatcctgaaagaacccgtgcacggagtgtactacgacccctccaaggacctgattgccgagatccagaaacagggacacgaccagtggacctaccagatctatcaggaacctttcaagaacctgaaaacaggcaagtacgccaagatgcggacagcccacaccaacgacgtgaagcagctgaccgaagccgtgcagaaaatcgccatggaaagcatcgtgatctggggaaagacacccaagttcaggctgcccatccagaaagagacatgggaaacctggtggaccgactactggcaggccacctggattcccgagtgggagttcgtgaacaccccacccctggtgaagctgtggtatcagctggaaaaggaccctatcgctggcgtggagacattctacgtggctggagctgccaacagagagacaaagctgggcaaggctggctacgtgaccgacagaggcagacagaaaatcgtgagcctgaccgaaaccaccaaccagaaaacagccctgcaggccatctatctggcactgcaggacagcggaagcgaggtgaacatcgtgacagccagccagtatgccctgggcatcatccaggcccagcctgacaagagcgagagcgagctggtgaaccagatcatcgagcagctgatcaagaaagaacgggtgtacctgagctgggtgccagcccacaagggcatcggagggaacgagcaggtggacaagctggtgtccagcggaatccggaaggtgctgttcctggacggcatcgataaagcccaggaagagcacgagaagtaccacagcaattggagagccatggccagcgacttcaacctgcctcccgtggtggccaaagaaatcgtggccagctgcgaccagtgccagctgaaaggcgaggccatgcacggacaggtggactgctcccctggcatctggcagctggcatgcacccacctggaaggcaagatcattctggtggccgtgcacgtggccagcggatacatcgaagccgaagtgatccctgccgagacagggcaggaaacagcctacttcatcctgaagctggctggcagatggcctgtgaaggtgatccacacagccaacggcagcaacttcacctctgctgccgtgaaggctgcctgttggtgggctggcattcagcaggaatttggcatcccctacaatccccagtctcagggagtggtggccagcatgaacaaagagctgaagaagatcatcggacaggtcagggatcaggccgagcacctgaaaactgccgtccagatggccgtgttcatccacaacttcaagcggaagggagggatcggagggtactctgctggcgagcggatcatcgacatcattgccaccgatatccagaccaaagagctgcagaaacagatcatcaagatccagaacttcagggtgtactacagggacagcagggaccccatctggaagggacctgccaagctgctgtggaaaggcgaaggagccgtcgtcatccaggacaacagcgacatcaaggtggtgcccagacggaaggtgaaaatcatcaaggactacggcaaacagatggctggagccgactgtgtcgctggcaggcaggacgaggac

SEQ ID NO: 8 - DNA, 4023 bp, Artificial Sequence
  mos2.GagPol nt sequence
atgggagccagagccagcatcctgcgaggagggaagctggacaagtgggagaagatcaggctgaggcctggagggaagaaacactacatgctgaagcacctggtctgggccagcagagagctggaacggtttgccctcaatcctggcctgctggaaaccagcgagggctgcaagcagatcatcaagcagctgcagcctgccctgcagacaggcaccgaggaactgcggagcctgttcaacaccgtggccaccctgtactgcgtgcatgccgagatcgaagtgagggacaccaaagaagccctggacaagatcgaggaagagcagaacaagagccagcagaaaacccagcaggccaaagaagccgacggcaaggtctcccagaactaccccatcgtgcagaacctgcagggacagatggtgcaccagcccatcagccctcggacactgaatgcctgggtgaaggtgatcgaggaaaaggccttcagccctgaggtgatccccatgttcacagccctgagcgagggagccacaccccaggacctgaacaccatgctgaacaccgtgggagggcaccaggctgccatgcagatgctgaaggacaccatcaacgaggaagctgccgagtgggacaggctgcaccctgtgcacgctggacctgtggctcctggccagatgagagagcccagaggcagcgatattgctggcaccacctccaatctgcaggaacagatcgcctggatgaccagcaaccctcccatccctgtgggagacatctacaagcggtggatcatcctgggactgaacaagatcgtgcggatgtacagccctacctccatcctggacatcaagcagggacccaaagagcctttcagggactacgtggaccggttcttcaagaccctgagagccgagcaggccacccaggacgtgaagaactggatgaccgacaccctgctggtgcagaacgccaaccctgactgcaagaccatcctgagagccctgggacctggagccaccctggaagagatgatgacagcctgccagggagtgggaggaccctctcacaaggctagggtgctggccgaggccatgagccagaccaacagcaccatcctgatgcagcggagcaacttcaagggcagcaagcggatcgtgaagtgcttcaactgtggcaaagagggacacattgccagaaactgtagggcacccaggaagaaaggctgctggaagtgcggaaaagaaggccaccagatgaaggactgcaccgagaggcaggccaacttcctgggcaagatctggcctagccacaagggcagacctggcaacttcctgcagagcagacccgagcccaccgctcctccagccgagagcttccggttcgaggaaaccacccctgctcccaagcaggaacctaaggacagagagcctctgaccagcctgagaagcctgttcggcagcgaccctctgagccagatggctcccatctcccctatcgagacagtgcctgtgaagctgaagcctggcatggacggacccaaggtgaaacagtggcctctgaccgaggaaaagatcaaagccctggtggagatctgtaccgagatggaaaaagagggcaagatcagcaagatcggacccgagaacccctacaacacccctatcttcgccatcaagaagaaagacagcaccaagtggaggaaactggtggacttcagagagctgaacaagcggacccaggacttctgggaggtgcagctgggcatccctcaccctgctggcctgaagaaaaagaaaagcgtgaccgtgctggccgtgggagatgcctacttcagcgtgcctctggacgaggacttcagaaagtacacagccttcaccatccccagcatcaacaacgagacacctggcatcagataccagtacaacgtgctgcctcagggatggaagggctctcctgcaatcttccagagcagcatgaccaagatcctggaacccttccggaagcagaaccctgacatcgtgatctaccagtacatggcagccctgtacgtcggcagcgacctggaaatcggacagcaccggaccaagatcgaagaactcaggcagcacctgctgcggtggggattcaccacccctgacaagaagcaccagaaagagcctcccttcctgtggatgggctacgagctgcacccagacaagtggaccgtgcagcccatcgtgctgcctgagaaggactcctggaccgtgaacgacatccagaaactggtcggcaagctgaactgggccagccagatctacgctggcatcaaagtgaagcagctgtgtaagctcctgagaggcaccaaagccctgaccgaggtggtgccactgacagaggaagccgagctggaactggccgagaacagagagatcctgaaagaacccgtgcacggagtgtactacgaccccagcaaggacctgattgccgagatccagaagcagggacagggacagtggacctaccagatctaccaggaacccttcaagaacctgaaaacaggcaagtacgccaggatgaggggagcccacaccaacgacgtcaaacagctgaccgaagccgtgcagaagatcgccaccgagagcatcgtgatttggggaaagacacccaagttcaagctgcccatccagaaagagacatgggaggcctggtggaccgagtactggcaggccacctggattcccgagtgggagttcgtgaacaccccacccctggtgaagctgtggtatcagctggaaaaagaacccatcgtgggagccgagacattctacgtggctggagctgccaacagagagacaaagctgggcaaggctggctacgtgaccgacagaggcaggcagaaagtggtgtccctgaccgataccaccaaccagaaaacagccctgcaggccatccacctggctctgcaggactctggcctggaagtgaacatcgtgacagccagccagtatgccctgggcatcattcaggcacagcctgacaagagcgagagcgagctggtgtctcagatcattgagcagctgatcaagaaagaaaaggtgtacctggcctgggtgccagcccacaaggggatcggagggaacgagcaggtggacaagctggtgtccaggggcatccggaaggtgctgtttctggacggcatcgacaaagcccaggaagagcacgagaagtaccacagcaattggagagccatggccagcgagttcaacctgcctcccatcgtggccaaagaaatcgtggcctcttgcgacaagtgccagctgaaaggcgaggccattcacggacaggtggactgcagcccaggcatctggcagctggcctgcacccacctggaaggcaaggtgatcctggtggccgtgcacgtggcctctggatacatcgaagccgaagtgatccctgccgagacaggccaggaaacagcctacttcctgctgaagctggctggcaggtggcctgtgaaaaccatccacacagccaacggcagcaacttcacctctgccaccgtgaaggctgcctgttggtgggctggcattaagcaggaatttggcatcccctacaaccctcagtctcagggagtggtggcctccatcaacaaagagctgaagaagatcatcggacaggtcagggatcaggccgagcatctgaaaacagccgtccagatggccgtgttcatccacaacttcaagcggaagggagggatcggagagtactctgctggcgagaggatcgtggacattatcgccagcgatatccagaccaaagaactgcagaagcagatcacaaagatccagaacttcagggtgtactacagggacagcagagatcccctgtggaagggacctgccaagctgctgtggaaaggcgaaggagccgtcgtcatccaggacaacagcgacatcaaggtggtgcccagacggaaggccaagatcatcagagactacggcaaacagatggctggcgacgactgcgtcgcctctaggcaggacgaggac

SEQ ID NO: 9 - DNA, 2055 bp, Artificial Sequence
  mos1.Env nt sequence
atgcgggtgaccggcatccggaagaactaccagcacctgtggcggtggggcaccatgctgctgggcatcctgatgatttgctctgccgccggaaagctgtgggtgaccgtgtactacggcgtgcccgtgtggaaagaggccaccaccaccctgttctgcgccagcgacgccaaggcctacgacaccgaggtgcacaacgtgtgggccacccacgcctgcgtgcccaccgaccccaacccccaggaagtggtcctggaaaacgtgaccgagaacttcaacatgtggaagaacaacatggtggagcagatgcacgaggacatcatcagcctgtgggaccagagcctgaagccctgcgtgaagctgacccccctgtgcgtgaccctgaactgcaccgacgacgtgcggaacgtgaccaacaacgccaccaacaccaacagcagctggggcgagcctatggaaaagggcgagatcaagaactgcagcttcaacatcaccacctccatccggaacaaggtgcagaagcagtacgccctgttctacaagctggacgtggtgcccatcgacaacgacagcaacaacaccaactaccggctgatcagctgcaacaccagcgtgatcacccaggcctgccccaaggtgtccttcgagcccatccccatccactactgcgcccctgccggcttcgccatcctgaagtgcaacgacaagaagttcaacggcaccggcccctgcaccaacgtgagcaccgtgcagtgcacccacggcatccggcccgtggtgtccacccagctgctgctgaacggcagcctggccgaggaagaggtggtgatcagaagcgagaatttcaccaacaatgccaagaccatcatggtgcagctgaacgtgagcgtggagatcaactgcacccggcccaacaacaacacccggaagagcatccacatcggccctggcagggccttctacacagccggcgacatcatcggcgacatccggcaggcccactgcaacatcagccgggccaactggaacaacaccctgcggcagatcgtggagaagctgggcaagcagttcggcaacaacaagaccatcgtgttcaaccacagcagcggcggagaccccgagatcgtgatgcacagcttcaactgtggcggcgagttcttctactgcaacagcaccaagctgttcaacagcacctggacctggaacaactccacctggaataacaccaagcggagcaacgacaccgaagagcacatcaccctgccctgccggatcaagcagattatcaatatgtggcaggaggtcggcaaggccatgtacgcccctcccatccggggccagatccggtgcagcagcaacatcaccggcctgctgctgacccgggacggcggcaacgataccagcggcaccgagatcttccggcctggcggcggagatatgcgggacaactggcggagcgagctgtacaagtacaaggtggtgaagatcgagcccctgggcgtggctcccaccaaggccaagcggcgggtggtgcagagcgagaagagcgccgtgggcatcggcgccgtgtttctgggcttcctgggagccgccggaagcaccatgggagccgccagcatgaccctgaccgtgcaggcccggctgctgctgtccggcatcgtgcagcagcagaacaacctgctccgggccatcgaggcccagcagcacctgctgcagctgaccgtgtggggcatcaagcagctgcaggccagggtgctggccgtggagagatacctgaaggatcagcagctcctggggatctggggctgcagcggcaagctgatctgcaccaccaccgtgccctggaacgccagctggtccaacaagagcctggacaagatctggaacaatatgacctggatggaatgggagcgcgagatcaacaattacaccagcctgatctacaccctgatcgaggaaagccagaaccagcaggaaaagaacgagcaggaactgctggaactggacaagtgggccagcctgtggaactggttcgacatcagcaactggctgtgg

SEQ ID NO: 10 - DNA, 2133 bp, Artificial Sequence
  mos2S.Env nt sequence
atgagagtgcggggcatgctgagaaactggcagcagtggtggatctggtccagcctgggcttctggatgctgatgatctacagcgtgatgggcaacctgtgggtcaccgtgtactacggcgtgcccgtgtggaaggacgccaagaccaccctgttttgcgcctccgatgccaaggcctacgagaaagaggtgcacaacgtctgggccacccacgcctgtgtgcccaccgaccccaatccccaggaaatcgtcctgggcaacgtgaccgagaacttcaacatgtggaagaacgacatggtcgatcagatgcacgaggacatcatctccctgtgggacgcctccctggaaccctgcgtgaagctgacccctctgtgcgtgaccctgaactgccggaacgtgcgcaacgtgtccagcaacggcacctacaacatcatccacaacgagacatacaaagagatgaagaactgcagcttcaacgctaccaccgtggtcgaggaccggaagcagaaggtgcacgccctgttctaccggctggacatcgtgcccctggacgagaacaacagcagcgagaagtcctccgagaacagctccgagtactacagactgatcaactgcaacaccagcgccatcacccaggcctgccccaaggtgtccttcgaccctatccccatccactactgcgcccctgccggctacgccatcctgaagtgcaacaacaagaccttcaatggcaccggcccctgcaacaatgtgtccaccgtgcagtgcacccacggcatcaagcccgtggtgtctacccagctgctgctgaacggcagcctggccgaggaagagatcattatcagaagcgagaacctgaccaacaacgccaaaaccatcatcgtccacctgaacgaaaccgtgaacatcacctgtacccggcctaacaacaacacccggaagtccatccggatcggccctggccagaccttttacgccaccggcgatattatcggcgacatccggcaggcccactgcaatctgagccgggacggctggaacaagacactgcagggcgtcaagaagaagctggccgaacacttccctaacaagactatcaagttcgcccctcactctggcggcgacctggaaatcaccacccacaccttcaactgtcggggcgagttcttctactgcaatacctccaacctgttcaacgagagcaacatcgagcggaacgacagcatcatcacactgccttgccggatcaagcagattatcaatatgtggcaggaagtgggcagagccatctacgcccctccaatcgccggcaacatcacatgccggtccaatatcaccggcctgctgctcaccagagatggcggctccaacaatggcgtgccaaacgacaccgagacattcagacccggcggaggcgacatgcggaacaattggcggagcgagctgtacaagtacaaggtggtggaagtgaagcccctgggcgtggcccctaccgaggccaagagaagagtggtcgaacgcgagaagcgggccgtgggaatcggagccgtgtttctgggaatcctgggagccgctggctctaccatgggcgctgcctctatcaccctgacagtgcaggccagacagctgctcagcggcatcgtgcagcagcagagcaacctgctgagagccattgaggcccagcagcacatgctgcagctgaccgtgtggggcattaagcagctccagacacgggtgctggccatcgagagatacctgcaggatcagcagctcctgggcctgtggggctgtagcggcaagctgatctgtaccaccgccgtgccctggaatacctcttggagcaacaagagccagaccgacatctgggacaacatgacctggatgcagtgggacaaagaaatcggcaactataccggcgagatctatagactgctggaagagtcccagaaccagcaggaaaagaacgagaaggacctgctggccctggattcttggaacaatctgtggaactggttcagcatctccaagtggctgtggtacatcaagatcttcatcatgatcgtgggcggcctgatcggcctgcggatcatctttgccgtgctgagcatcgtgaaccgcgtgcggcagggctac

SEQ ID NO: 11 - protein, 1350 aa, Artificial Sequence
  mos1.GagPol
MGARASVLSGGELDRWEKIRLRPGGKKKYRLKHIVWASRELERFAVNPGLLETSEGCRQILGQLQPSLQTGSEELRSLYNTVATLYCVHQRIEIKDTKEALEKIEEEQNKSKKKAQQAAADTGNSSQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPVSILDIRQGPKEPFRDYVDRFYKTLRAEQASQDVKNWMTETLLVQNANPDCKTILKALGPAATLEEMMTACQGVGGPGHKARVLAEAMSQVTNSATIMMQRGNFRNQRKTVKCFNCGKEGHIAKNCRAPRKKGCWKCGKEGHQMKDCTERQANFLGKIWPSNKGRPGNFLQNRPEPTAPPEESFRFGEETTTPSQKQEPIDKEMYPLASLKSLFGNDPSSQMAPISPIETVPVKLKPGMDGPRVKQWPLTEEKIKALTAICEEMEKEGKITKIGPENPYNTPVFAIKKKDSTKWRKLVDFRELNKRTQDFWEVQLGIPHPAGLKKKKSVTVLAVGDAYFSVPLDEGFRKYTAFTIPSTNNETPGIRYQYNVLPQGWKGSPAIFQCSMTRILEPFRAKNPEIVIYQYMAALYVGSDLEIGQHRAKIEELREHLLKWGFTTPDKKHQKEPPFLWMGYELHPDKWTVQPIQLPEKDSWTVNDIQKLVGKLNWASQIYPGIKVRQLCKLLRGAKALTDIVPLTEEAELELAENREILKEPVHGVYYDPSKDLIAEIQKQGHDQWTYQIYQEPFKNLKTGKYAKMRTAHTNDVKQLTEAVQKIAMESIVIWGKTPKFRLPIQKETWETWWTDYWQATWIPEWEFVNTPPLVKLWYQLEKDPIAGVETFYVAGAANRETKLGKAGYVTDRGRQKIVSLTETTNQKTALQAIYLALQDSGSEVNIVTASQYALGIIQAQPDKSESELVNQIIEQLIKKERVYLSWVPAHKGIGGNEQVDKLVSSGIRKVLFLDGIDKAQEEHEKYHSNWRAMASDFNLPPVVAKEIVASCDQCQLKGEAMHGQVDCSPGIWQLACTHLEGKIILVAVHVASGYIEAEVIPAETGQETAYFILKLAGRWPVKVIHTANGSNFTSAAVKAACWWAGIQQEFGIPYNPQSQGVVASMNKELKKIIGQVRDQAEHLKTAVQMAVFIHNFKRKGGIGGYSAGERIIDIIATDIQTKELQKQIIKIQNFRVYYRDSRDPIWKGPAKLLWKGEGAVVIQDNSDIKVVPRRKVKIIKDYGKQMAGADCVAGRQDED

SEQ ID NO: 12 - protein, 1341 aa, Artificial Sequence
  mos2.GagPol
MGARASILRGGKLDKWEKIRLRPGGKKHYMLKHLVWASRELERFALNPGLLETSEGCKQIIKQLQPALQTGTEELRSLFNTVATLYCVHAEIEVRDTKEALDKIEEEQNKSQQKTQQAKEADGKVSQNYPIVQNLQGQMVHQPISPRTLNAWVKVIEEKAFSPEVIPMFTALSEGATPQDLNTMLNTVGGHQAAMQMLKDTINEEAAEWDRLHPVHAGPVAPGQMREPRGSDIAGTTSNLQEQIAWMTSNPPIPVGDIYKRWIILGLNKIVRMYSPTSILDIKQGPKEPFRDYVDRFFKTLRAEQATQDVKNWMTDTLLVQNANPDCKTILRALGPGATLEEMMTACQGVGGPSHKARVLAEAMSQTNSTILMQRSNFKGSKRIVKCFNCGKEGHIARNCRAPRKKGCWKCGKEGHQMKDCTERQANFLGKIWPSHKGRPGNFLQSRPEPTAPPAESFRFEETTPAPKQEPKDREPLTSLRSLFGSDPLSQMAPISPIETVPVKLKPGMDGPKVKQWPLTEEKIKALVEICTEMEKEGKISKIGPENPYNTPIFAIKKKDSTKWRKLVDFRELNKRTQDFWEVQLGIPHPAGLKKKKSVTVLAVGDAYFSVPLDEDFRKYTAFTIPSINNETPGIRYQYNVLPQGWKGSPAIFQSSMTKILEPFRKQNPDIVIYQYMAALYVGSDLEIGQHRTKIEELRQHLLRWGFTTPDKKHQKEPPFLWMGYELHPDKWTVQPIVLPEKDSWTVNDIQKLVGKLNWASQIYAGIKVKQLCKLLRGTKALTEVVPLTEEAELELAENREILKEPVHGVYYDPSKDLIAEIQKQGQGQWTYQIYQEPFKNLKTGKYARMRGAHTNDVKQLTEAVQKIATESIVIWGKTPKFKLPIQKETWEAWWTEYWQATWIPEWEFVNTPPLVKLWYQLEKEPIVGAETFYVAGAANRETKLGKAGYVTDRGRQKVVSLTDTTNQKTALQAIHLALQDSGLEVNIVTASQYALGIIQAQPDKSESELVSQIIEQLIKKEKVYLAWVPAHKGIGGNEQVDKLVSRGIRKVLFLDGIDKAQEEHEKYHSNWRAMASEFNLPPIVAKEIVASCDKCQLKGEAIHGQVDCSPGIWQLACTHLEGKVILVAVHVASGYIEAEVIPAETGQETAYFLLKLAGRWPVKTIHTANGSNFTSATVKAACWWAGIKQEFGIPYNPQSQGVVASINKELKKIIGQVRDQAEHLKTAVQMAVFIHNFKRKGGIGEYSAGERIVDIIASDIQTKELQKQITKIQNFRVYYRDSRDPLWKGPAKLLWKGEGAVVIQDNSDIKVVPRRKAKIIRDYGKQMAGDDCVASRQDED

SEQ ID NO: 13 - protein, 15 aa, Artificial Sequence
  Env peptide
KEIRKNYQHLWRWGT

SEQ ID NO: 14 - protein, 15 aa, Artificial Sequence
  env peptide
MNWPNLWKWGTLILG

SEQ ID NO: 15 - protein, 15 aa, Artificial Sequence
  env peptide
YQHLWRWGTMLLGML

SEQ ID NO: 16 - protein, 15 aa, Artificial Sequence
  env peptide
PQWWIWGILGFWMLM

SEQ ID NO: 17 - protein, 15 aa, Artificial Sequence
  env peptide
WKWGTLILGLVIICS

SEQ ID NO: 18 - protein, 15 aa, Artificial Sequence
  env peptide
WGTMLLGMLMICSAA

SEQ ID NO: 19 - protein, 15 aa, Artificial Sequence
  env peptide
GILGFWMLMICNVVG

SEQ ID NO: 20 - protein, 15 aa, Artificial Sequence
  env peptide
ILGLVIICSASDNLW

SEQ ID NO: 21 - protein, 15 aa, Artificial Sequence
  env peptide
LGMLMICSATEKLWV

SEQ ID NO: 22 - protein, 15 aa, Artificial Sequence
  env peptide
GMLMICSAAEQLWVT

SEQ ID NO: 23 - protein, 15 aa, Artificial Sequence
  env peptide
ICNVVGNLWVTVYYG

SEQ ID NO: 24 - protein, 15 aa, Artificial Sequence
  env peptide
ICSAAENLWVTVYYG

SEQ ID NO: 25 - protein, 15 aa, Artificial Sequence
  env peptide
ICSASDNLWVTVYYG

SEQ ID NO: 26 - protein, 15 aa, Artificial Sequence
  env peptide
CSATEKLWVTVYYGV

SEQ ID NO: 27 - protein, 15 aa, Artificial Sequence
  env peptide
CSAAEQLWVTVYYGV

SEQ ID NO: 28 - protein, 15 aa, Artificial Sequence
  env peptide
NLWVTVYYGVPVWKE

SEQ ID NO: 29 - protein, 15 aa, Artificial Sequence
  env peptide
TVYYGVPVWRDAETT

SEQ ID NO: 30 - protein, 15 aa, Artificial Sequence
  env peptide
VYYGVPVWKDAETTL

SEQ ID NO: 31 - protein, 15 aa, Artificial Sequence
  env peptide
VYYGVPVWREAKTTL

SEQ ID NO: 32 - protein, 15 aa, Artificial Sequence
  env peptide
YYGVPVWKEATTTLF

SEQ ID NO: 33 - protein, 15 aa, Artificial Sequence
  env peptide
YGVPVWKEAKTTLLC

SEQ ID NO: 34 - protein, 15 aa, Artificial Sequence
  env peptide
PVWRDAETTLFCASE

SEQ ID NO: 35 - protein, 15 aa, Artificial Sequence
  env peptide
VWKEAKTTLFCASDA

SEQ ID NO: 36 - protein, 15 aa, Artificial Sequence
  env peptide
VWKDAETTLFCASDA

SEQ ID NO: 37 - protein, 15 aa, Artificial Sequence
  env peptide
VWREAKTTLFCASNA

SEQ ID NO: 38 - protein, 15 aa, Artificial Sequence
  env peptide
WKEATTTLFCASDAR

SEQ ID NO: 39 - protein, 15 aa, Artificial Sequence
  env peptide
DADTTLFCASDAKSY

SEQ ID NO: 40 - protein, 15 aa, Artificial Sequence
  env peptide
ATTTLFCASDAKAYD

SEQ ID NO: 41 - protein, 15 aa, Artificial Sequence
  env peptide
LLCASDAKAYEREVH

SEQ ID NO: 42 - protein, 15 aa, Artificial Sequence
  env peptide
FCASDAKAYEKEVHN

SEQ ID NO: 43 - protein, 15 aa, Artificial Sequence
  env peptide
CASDAKAYDTEVHNV

SEQ ID NO: 44 - protein, 15 aa, Artificial Sequence
  env peptide
ASDARAYDTEVHNVW

SEQ ID NO: 45 - protein, 15 aa, Artificial Sequence
  env peptide
AKAYEKEVHNVWATH

SEQ ID NO: 46 - protein, 15 aa, Artificial Sequence
  env peptide
AKAYEREVHNVWATH

SEQ ID NO: 47 - protein, 15 aa, Artificial Sequence
  env peptide
AYETEKHNVWATHAC

SEQ ID NO: 48 - protein, 15 aa, Artificial Sequence
  env peptide
AYDTEAHNVWATHAC

SEQ ID NO: 49 - protein, 15 aa, Artificial Sequence
  env peptide
YDTEVHNVWATHACV

SEQ ID NO: 50 - protein, 15 aa, Artificial Sequence
  env peptide
TEAHNIWATHACVPT

SEQ ID NO: 51 - protein, 15 aa, Artificial Sequence
  env peptide
NVWATHACVPTDPNP

SEQ ID NO: 52 - protein, 15 aa, Artificial Sequence
  env peptide
ACVPTDPNPQEIHLE

SEQ ID NO: 53 - protein, 15 aa, Artificial Sequence
  env peptide
CIPTDPNPQEIVLEN

SEQ ID NO: 54 - protein, 15 aa, Artificial Sequence
  env peptide
VPTDPNPQEVVLGNV

SEQ ID NO: 55 - protein, 15 aa, Artificial Sequence
  env peptide
VPTDPNPQEMVLENV

SEQ ID NO: 56 - protein, 15 aa, Artificial Sequence
  env peptide
PNPQEIHLENVTEEF

SEQ ID NO: 57 - protein, 15 aa, Artificial Sequence
  env peptide
NPQEIVLENVTENFN

SEQ ID NO: 58 - protein, 15 aa, Artificial Sequence
  env peptide
PQEINLENVTEEFNM

SEQ ID NO: 59 - protein, 15 aa, Artificial Sequence
  env peptide
VVLGNVTENFNMWEN

SEQ ID NO: 60 - protein, 15 aa, Artificial Sequence
  env peptide
LENVTENFNMWKNNM

SEQ ID NO: 61 - protein, 15 aa, Artificial Sequence
  env peptide
ENVTEEFNMWKNNMV

SEQ ID NO: 62 - protein, 15 aa, Artificial Sequence
  env peptide
TENFNMWKNDMVDQM

SEQ ID NO: 63 - protein, 15 aa, Artificial Sequence
  env peptide
NFNMWKNNMVEQMHE

SEQ ID NO: 64 - protein, 15 aa, Artificial Sequence
  env peptide
NFDMWKNNMVEQMHT

SEQ ID NO: 65 - protein, 15 aa, Artificial Sequence
  env peptide
MKNNMVEQMHEDIIS

SEQ ID NO: 66 - protein, 15 aa, Artificial Sequence
  env peptide
WKNDMVDQMHEDVIS

SEQ ID NO: 67 - protein, 15 aa, Artificial Sequence
  env peptide
KNDMVDQMHEDIISL

SEQ ID NO: 68 - protein, 15 aa, Artificial Sequence
  env peptide
NNMVEQMHTDIISLW

SEQ ID NO: 69 - protein, 15 aa, Artificial Sequence
  env peptide
NDMVNQMHEDVISLW

SEQ ID NO: 70 - protein, 15 aa, Artificial Sequence
  env peptide
EQMHEDIISLWDQSL

SEQ ID NO: 71 - protein, 15 aa, Artificial Sequence
  env peptide
HMHEDIISLWDESLK

SEQ ID NO: 72 - protein, 15 aa, Artificial Sequence
  env peptide
MHTDIISLWDQSLKP

SEQ ID NO: 73 - protein, 15 aa, Artificial Sequence
  env peptide
MHEDVISLWDQSLKP

SEQ ID NO: 74 - protein, 15 aa, Artificial Sequence
  env peptide
ISLWDQSLKPCVKLT

SEQ ID NO: 75 - protein, 15 aa, Artificial Sequence
  env peptide
ISLWDESLKPCVKLT

SEQ ID NO: 76 - protein, 15 aa, Artificial Sequence
  env peptide
SLKPCVKLTPLCVTL

SEQ ID NO: 77 - protein, 15 aa, Artificial Sequence
  env peptide
CVQLTPLCVTLNCSN

SEQ ID NO: 78 - protein, 15 aa, Artificial Sequence
  env peptide
KLTPLCVTLNCTDVK

SEQ ID NO: 79 - protein, 15 aa, Artificial Sequence
  env peptide
KLTPLCVTLDCTNAT

SEQ ID NO: 80 - protein, 15 aa, Artificial Sequence
  env peptide
PLCVTLNCTNANITS

SEQ ID NO: 81 - protein, 15 aa, Artificial Sequence
  env peptide
LCVTLNCTDLRNTTN

SEQ ID NO: 82 - protein, 15 aa, Artificial Sequence
  env peptide
MEKGEIKNCSFNVTT

SEQ ID NO: 83 - protein, 15 aa, Artificial Sequence
  env peptide
KGEIKNCSFNITTSI

SEQ ID NO: 84 - protein, 15 aa, Artificial Sequence
  env peptide
GEIKNCSFNMTTEVR

SEQ ID NO: 85 - protein, 15 aa, Artificial Sequence
  env peptide
EMKNCSFNITTELRD

SEQ ID NO: 86 - protein, 15 aa, Artificial Sequence
  env peptide
EIKNCSFNTTTEIRD

SEQ ID NO: 87 - protein, 15 aa, Artificial Sequence
  env peptide
IKNCSFNMTTELRDK

SEQ ID NO: 88 - protein, 15 aa, Artificial Sequence
  env peptide
CSFNITTSIRDKVQK

SEQ ID NO: 89 - protein, 15 aa, Artificial Sequence
  env peptide
NMTTELRDKKQKVYS

SEQ ID NO: 90 - protein, 15 aa, Artificial Sequence
  env peptide
TSIRDKVQKEYALFY

SEQ ID NO: 91 - protein, 15 aa, Artificial Sequence
  env peptide
KEYALFYKLDVVPID

SEQ ID NO: 92 - protein, 15 aa, Artificial Sequence
  env peptide
KEYALFYRLDIVPLN

SEQ ID NO: 93 - protein, 15 aa, Artificial Sequence
  env peptide
KVYALFYKLDIVPLN

SEQ ID NO: 94 - protein, 15 aa, Artificial Sequence
  env peptide
KVYSLFYRLDVVPID

SEQ ID NO: 95 - protein, 15 aa, Artificial Sequence
  env peptide
YSLFYKLDVVPIDND

SEQ ID NO: 96 - protein, 15 aa, Artificial Sequence
  env peptide
SEYRLINCNTSTITQ

SEQ ID NO: 97 - protein, 15 aa, Artificial Sequence
  env peptide
TSYRLINCNTSVITQ

SEQ ID NO: 98 - protein, 15 aa, Artificial Sequence
  env peptide
TSYRLISCNTSVITQ

SEQ ID NO: 99 - protein, 15 aa, Artificial Sequence
  env peptide
SQYRLINCNTSAVTQ

SEQ ID NO: 100 - protein, 15 aa, Artificial Sequence
  env peptide
YRLINCNTSAITQAC

SEQ ID NO: 101 - protein, 15 aa, Artificial Sequence
  env peptide
ILINCNTSVIKQACP

SEQ ID NO: 102 - protein, 15 aa, Artificial Sequence
  env peptide
SCNTSVITQACPKVS

SEQ ID NO: 103 - protein, 15 aa, Artificial Sequence
  env peptide
NCNTSTITQACPKVS

SEQ ID NO: 104 - protein, 15 aa, Artificial Sequence
  env peptide
NTSAITQACPKVTFE

SEQ ID NO: 105 - protein, 15 aa, Artificial Sequence
  env peptides
NVSTIKQACPKVSFE

SEQ ID NO: 106 - protein, 15 aa, Artificial Sequence
  env peptide
TSVIKQACPKVTFEP

SEQ ID NO: 107 - protein, 15 aa, Artificial Sequence
  env peptide
SAVTQACPKVTFDPI

SEQ ID NO: 108 - protein, 15 aa, Artificial Sequence
  env peptide
AITQACPKVSFDPIP

SEQ ID NO: 109 - protein, 15 aa, Artificial Sequence
  env peptide
ITQACPKVSFEPIPI

SEQ ID NO: 110 - protein, 15 aa, Artificial Sequence
  env peptide
TQACPKVSWDPIPIH

SEQ ID NO: 111 - protein, 15 aa, Artificial Sequence
  env peptide
ACPKVTFEPIPIHYC

SEQ ID NO: 112 - protein, 15 aa, Artificial Sequence
  env peptide
CPKVTFDPIPIHYCT

SEQ ID NO: 113 - protein, 15 aa, Artificial Sequence
  env peptide
PKVSFDPIPIHYCAP

SEQ ID NO: 114 - protein, 15 aa, Artificial Sequence
  env peptide
KVSFEPIPIHYCTPA

SEQ ID NO: 115 - protein, 15 aa, Artificial Sequence
  env peptide
SWDPIPIHYCAPAGY

SEQ ID NO: 116 - protein, 15 aa, Artificial Sequence
  env peptide
FEPIPIHYCAPAGFA

SEQ ID NO: 117 - protein, 15 aa, Artificial Sequence
  env peptide
DPIPIHYCTPAGFAI

SEQ ID NO: 118 - protein, 15 aa, Artificial Sequence
  env peptide
IHYCAPAGYAILKCN

SEQ ID NO: 119 - protein, 15 aa, Artificial Sequence
  env peptide
HYCAPAGFAILKCND

SEQ ID NO: 120 - protein, 15 aa, Artificial Sequence
  env peptide
YCTPAGFAILKCKDK

SEQ ID NO: 121 - protein, 15 aa, Artificial Sequence
  env peptide
CTPAGYAILKCNNKT

SEQ ID NO: 122 - protein, 15 aa, Artificial Sequence
  env peptide
PAGFAILKCRDKKFN

SEQ ID NO: 123 - protein, 15 aa, Artificial Sequence
  env peptide
AGFAILKCNNKTFSG

SEQ ID NO: 124 - protein, 15 aa, Artificial Sequence
  env peptide
AGYAILKCNDKNFNG

SEQ ID NO: 125 - protein, 15 aa, Artificial Sequence
  env peptide
GFAILKCNDKKFNGT

SEQ ID NO: 126 - protein, 15 aa, Artificial Sequence
  env peptide
AILKCNNKTFNGTGP

SEQ ID NO: 127 - protein, 15 aa, Artificial Sequence
  env peptide
CNDKKFNGTGLCKNV

SEQ ID NO: 128 - protein, 15 aa, Artificial Sequence
  env peptide
NDKKFNGTGPCKNVS

SEQ ID NO: 129 - protein, 15 aa, Artificial Sequence
  env peptide
NNKKFNGTGPCTNVS

SEQ ID NO: 130 - protein, 15 aa, Artificial Sequence
  env peptide
NKTFNGTGPCNNVST

SEQ ID NO: 131 - protein, 15 aa, Artificial Sequence
  env peptide
GTGPCKNVSTVQCTH

SEQ ID NO: 132 - protein, 15 aa, Artificial Sequence
  env peptide
GTGPCTNVSTVQCTH

SEQ ID NO: 133 - protein, 15 aa, Artificial Sequence
  env peptide
GTGPCHNVSTVQCTH

SEQ ID NO: 134 - protein, 15 aa, Artificial Sequence
  env peptide
TGPCNNVSTVQCTHG

SEQ ID NO: 135 - protein, 15 aa, Artificial Sequence
  env peptide
CKNVSSVQCTHGIKP

SEQ ID NO: 136 - protein, 15 aa, Artificial Sequence
  env peptide
NVSTVQCTHGIKPVV

SEQ ID NO: 137 - protein, 15 aa, Artificial Sequence
  env peptide
NISTVQCTHGIRPVV

SEQ ID NO: 138 - protein, 15 aa, Artificial Sequence
  env peptide
CTHGIKPVVSTQLLL

SEQ ID NO: 139 - protein, 15 aa, Artificial Sequence
  env peptide
CTHGIRPVVSTQLLL

SEQ ID NO: 140 - protein, 15 aa, Artificial Sequence
  env peptide
PVVSTQLLLNGSLAE

SEQ ID NO: 141 - protein, 15 aa, Artificial Sequence
  env peptide
LLLNGSLAEEEIIIR

SEQ ID NO: 142 - protein, 15 aa, Artificial Sequence
  env peptide
LLLNGSLAEGEIIIR

SEQ ID NO: 143 - protein, 15 aa, Artificial Sequence
  env peptide
LLLNGSLAEKEIIIR

SEQ ID NO: 144 - protein, 15 aa, Artificial Sequence
  env peptide
LNGSLAEEEVVIRSE

SEQ ID NO: 145 - protein, 15 aa, Artificial Sequence
  env peptide
LAEEEIIIRSENITN

SEQ ID NO: 146 - protein, 15 aa, Artificial Sequence
  env peptide
LAEGEIIIRSQNISD

SEQ ID NO: 147 - protein, 15 aa, Artificial Sequence
  env peptide
AEEEIIIRSENLTNN

SEQ ID NO: 148 - protein, 15 aa, Artificial Sequence
  env peptide
AEGEIIIRSENLTDN

SEQ ID NO: 149 - protein, 15 aa, Artificial Sequence
  env peptide
AEEEVMIRSENITNN

SEQ ID NO: 150 - protein, 15 aa, Artificial Sequence
  env peptide
EIVIRSENLTNNVKT

SEQ ID NO: 151 - protein, 15 aa, Artificial Sequence
  env peptide
IRSENITNNAKTIIV

SEQ ID NO: 152 - protein, 15 aa, Artificial Sequence
  env peptide
IRSENLTNNAKIIIV

SEQ ID NO: 153 - protein, 15 aa, Artificial Sequence
  env peptide
IRSENFTNNAKTIIV

SEQ ID NO: 154 - protein, 15 aa, Artificial Sequence
  env peptide
SENLTDNVKTIIVHL

SEQ ID NO: 155 - protein, 15 aa, Artificial Sequence
  env peptide
SENITNNAKNIIVQL

SEQ ID NO: 156 - protein, 15 aa, Artificial Sequence
  env peptide
ENLTNNAKTIIVQLN

SEQ ID NO: 157 - protein, 15 aa, Artificial Sequence
  env peptide
ENLTNNVKTIIVHLN

SEQ ID NO: 158 - protein, 15 aa, Artificial Sequence
  env peptide
NFTDNAKTIIVQLKE

SEQ ID NO: 159 - protein, 15 aa, Artificial Sequence
  env peptide
TNNAKTIIVHLNESV

SEQ ID NO: 160 - protein, 15 aa, Artificial Sequence
  env peptide
AKTIIVQLNESVEIN

SEQ ID NO: 161 - protein, 15 aa, Artificial Sequence
  env peptide
VKTIIVHLNESIEIV

SEQ ID NO: 162 - protein, 15 aa, Artificial Sequence
  env peptide
IIVHLNESVEIVCTR

SEQ ID NO: 163 - protein, 15 aa, Artificial Sequence
  env peptide
HLNQSVEIVCTRPNN

SEQ ID NO: 164 - protein, 15 aa, Artificial Sequence
  env peptide
LNESVEINCTRPNNN

SEQ ID NO: 165 - protein, 15 aa, Artificial Sequence
  env peptide
EIVCTRPNNNTRKGI

SEQ ID NO: 166 - protein, 15 aa, Artificial Sequence
  env peptide
INCTRPNNNTRKSIR

SEQ ID NO: 167 - protein, 15 aa, Artificial Sequence
  env peptide
INCTRPGNNTRKSIR

SEQ ID NO: 168 - protein, 15 aa, Artificial Sequence
  env peptide
TCIRPNNNTRKSVRI

SEQ ID NO: 169 - protein, 15 aa, Artificial Sequence
  env peptide
PNNNTRKSIHIGPGR

SEQ ID NO: 170 - protein, 15 aa, Artificial Sequence
  env peptide
NNNTRKSIRIGPGQT

SEQ ID NO: 171 - protein, 15 aa, Artificial Sequence
  env peptide
NNTRKSVRIGPGQTF

SEQ ID NO: 172 - protein, 15 aa, Artificial Sequence
  env peptide
KSIHIGPGRAFYATG

SEQ ID NO: 173 - protein, 15 aa, Artificial Sequence
  env peptide
SIRIGPGQTFYATGD

SEQ ID NO: 174 - protein, 15 aa, Artificial Sequence
  env peptide
VRIGPGQTFYATGEI

SEQ ID NO: 175 - protein, 15 aa, Artificial Sequence
  env peptide
RIGPGQAFYATGDII

SEQ ID NO: 176 - protein, 15 aa, Artificial Sequence
  env peptide
GQTFYATGDIIGNIR

SEQ ID NO: 177 - protein, 15 aa, Artificial Sequence
  env peptide
TGDIIGDIRKAHCNV

SEQ ID NO: 178 - protein, 15 aa, Artificial Sequence
  env peptide
TGEIIGDIRQAHCNL

SEQ ID NO: 179 - protein, 15 aa, Artificial Sequence
  env peptide
GDIIGDIRQAHCNIS

SEQ ID NO: 180 - protein, 15 aa, Artificial Sequence
  env peptide
ITGDIRQAHCNVSRS

SEQ ID NO: 181 - protein, 15 aa, Artificial Sequence
  env peptide
DIRQAHCNLSRAKWN

SEQ ID NO: 182 - protein, 15 aa, Artificial Sequence
  env peptide
EQFGNKTIVFNQSSG

SEQ ID NO: 183 - protein, 15 aa, Artificial Sequence
  env peptide
TIVFNQSSGGDPEIV

SEQ ID NO: 184 - protein, 15 aa, Artificial Sequence
  env peptide
TIIFTNSSGGDLEIT

SEQ ID NO: 185 - protein, 15 aa, Artificial Sequence
  env peptide
TIIFKPSSGGDLEIT

SEQ ID NO: 186 - protein, 15 aa, Artificial Sequence
  env peptide
IIFNSSSGGDLEITT

SEQ ID NO: 187 - protein, 15 aa, Artificial Sequence
  env peptide
FKPSSGGDPEITTHS

SEQ ID NO: 188 - protein, 15 aa, Artificial Sequence
  env peptide
FAPSSGGDLEVTTHS

SEQ ID NO: 189 - protein, 15 aa, Artificial Sequence
  env peptide
FQPPSGGDLEITMHH

SEQ ID NO: 190 - protein, 15 aa, Artificial Sequence
  env peptide
SSGGDPEIVMHSFNC

SEQ ID NO: 191 - protein, 15 aa, Artificial Sequence
  env peptide
SGGDLEITTHSFNCG

SEQ ID NO: 192 - protein, 15 aa, Artificial Sequence
  env sequence
GDPEITTHSFNCRGE

SEQ ID NO: 193 - protein, 15 aa, Artificial Sequence
  env peptide
GDIEITTHSFNCGGE

SEQ ID NO: 194 - protein, 15 aa, Artificial Sequence
  env peptide
DLEIVMHSFNCGGEF

SEQ ID NO: 195 - protein, 15 aa, Artificial Sequence
  env peptide
THSFNCGGEFFYCNT

SEQ ID NO: 196 - protein, 15 aa, Artificial Sequence
  env peptide
THSFNCRGEFFYCNT

SEQ ID NO: 197 - protein, 15 aa, Artificial Sequence
  env peptide
CGGEFFYCNSTQLFN

SEQ ID NO: 198 - protein, 15 aa, Artificial Sequence
  env peptide
GGEFFYCNTSGLFNS

SEQ ID NO: 199 - protein, 15 aa, Artificial Sequence
  env peptide
RGEFFYCNTSKLFNS

SEQ ID NO: 200 - protein, 15 aa, Artificial Sequence
  env peptide
GGEFFYCNTTQLFNS

SEQ ID NO: 201 - protein, 15 aa, Artificial Sequence
  env peptide
RGEFFYCNTTKLFNS

SEQ ID NO: 202 - protein, 15 aa, Artificial Sequence
  env peptide
YCNSTQLFNSTWNST

SEQ ID NO: 203 - protein, 15 aa, Artificial Sequence
  env peptide
CNTSGLFNSTWNDTG

SEQ ID NO: 204 - protein, 15 aa, Artificial Sequence
  env peptide
NSTITIPCRIKQIIN

SEQ ID NO: 205 - protein, 15 aa, Artificial Sequence
  env peptide
DTITLQCRIKQIINM

SEQ ID NO: 206 - protein, 15 aa, Artificial Sequence
  env peptide
TITLPCRIKQIVNMW

SEQ ID NO: 207 - protein, 15 aa, Artificial Sequence
  env peptide
TIILPCRIKQIINRW

SEQ ID NO: 208 - protein, 15 aa, Artificial Sequence
  env peptide
ITLPCRIKQIINMWQ

SEQ ID NO: 209 - protein, 15 aa, Artificial Sequence
  env peptide
CKIKQIINMWQGVGR

SEQ ID NO: 210 - protein, 15 aa, Artificial Sequence
  env peptide
RIKQIVNMWQEVGRA

SEQ ID NO: 211 - protein, 15 aa, Artificial Sequence
  env peptide
RIRQIINMWQEVGKA

SEQ ID NO: 212 - protein, 15 aa, Artificial Sequence
  env peptide
IKQIINMWQEVGRAM

SEQ ID NO: 213 - protein, 15 aa, Artificial Sequence
  env peptide
IKQIINMWQRVGQAM

SEQ ID NO: 214 - protein, 15 aa, Artificial Sequence
  env peptide
INMWQGVGRAMYAPP

SEQ ID NO: 215 - protein, 15 aa, Artificial Sequence
  env peptide
NMWQEVGKAMYAPPI

SEQ ID NO: 216 - protein, 15 aa, Artificial Sequence
  env peptide
MWQEVGRAMYAPPIA

SEQ ID NO: 217 - protein, 15 aa, Artificial Sequence
  env peptide
MWQRVGQAMYAPPIQ

SEQ ID NO: 218 - protein, 15 aa, Artificial Sequence
  env peptide
RAGQAMYAPPIPGVI

SEQ ID NO: 219 - protein, 15 aa, Artificial Sequence
  env peptide
GRAMYAPPIEGNITC

SEQ ID NO: 220 - protein, 15 aa, Artificial Sequence
  env peptide
GKAMYAPPISGQIRC

SEQ ID NO: 221 - protein, 15 aa, Artificial Sequence
  env peptide
GKAMYAPPIRGQIRC

SEQ ID NO: 222 - protein, 15 aa, Artificial Sequence
  env peptide
GQAMYAPPIQGVIRC

SEQ ID NO: 223 - protein, 15 aa, Artificial Sequence
  env peptide
RAMYAPPIAGNITCK

SEQ ID NO: 224 - protein, 15 aa, Artificial Sequence
  env peptide
APPIEGNITCKSNIT

SEQ ID NO: 225 - protein, 15 aa, Artificial Sequence
  env peptide
PPISGQIRCSSKITG

SEQ ID NO: 226 - protein, 15 aa, Artificial Sequence
  env peptide
PIAGNITCKSNITGI

SEQ ID NO: 227 - protein, 15 aa, Artificial Sequence
  env peptide
ISGQIRCSSNITGLL

SEQ ID NO: 228 - protein, 15 aa, Artificial Sequence
  env peptide
GNITCKSNITGLLLT

SEQ ID NO: 229 - protein, 15 aa, Artificial Sequence
  env peptide
GVIRCESNITGLLLT

SEQ ID NO: 230 - protein, 15 aa, Artificial Sequence
  env peptide
GRIICKSNITGLLLV

SEQ ID NO: 231 - protein, 15 aa, Artificial Sequence
  env peptide
CSSNITGLLLTRDGG

SEQ ID NO: 232 - protein, 15 aa, Artificial Sequence
  env peptide
CTSNITGLLLVRDGG

SEQ ID NO: 233 - protein, 15 aa, Artificial Sequence
  env peptide
SSNITGLILTRDGGN

SEQ ID NO: 234 - protein, 15 aa, Artificial Sequence
  env peptide
GLLLTRDGGNNNNGS

SEQ ID NO: 235 - protein, 15 aa, Artificial Sequence
  env peptide
STNETFRPGGGDMRD

SEQ ID NO: 236 - protein, 15 aa, Artificial Sequence
  env peptide
NDTEIFRPGGGDMRN

SEQ ID NO: 237 - protein, 15 aa, Artificial Sequence
  env peptide
NETFRPGGGNMKDNW

SEQ ID NO: 238 - protein, 15 aa, Artificial Sequence
  env peptide
TEIFRPGGGNMRDNW

SEQ ID NO: 239 - protein, 15 aa, Artificial Sequence
  env peptide
TETFRPGGGDMKDNW

SEQ ID NO: 240 - protein, 15 aa, Artificial Sequence
  env peptide
RPGGGDMRDNWRSEL

SEQ ID NO: 241 - protein, 15 aa, Artificial Sequence
  env peptide
GGGNMKDNWRSELYK

SEQ ID NO: 242 - protein, 15 aa, Artificial Sequence
  env peptide
MRDNWRSELYKYKVV

SEQ ID NO: 243 - protein, 15 aa, Artificial Sequence
  env peptide
SELYKYKVVKIEPLG

SEQ ID NO: 244 - protein, 15 aa, Artificial Sequence
  env peptide
SELYKYKVVEIKPLG

SEQ ID NO: 245 - protein, 15 aa, Artificial Sequence
  env peptide
SELYKYKVVRIEPLG

SEQ ID NO: 246 - protein, 15 aa, Artificial Sequence
  env peptide
LYKYKVVEIEPLGVA

SEQ ID NO: 247 - protein, 15 aa, Artificial Sequence
  env peptide
YKVVQIEPLGVAPTR

SEQ ID NO: 248 - protein, 15 aa, Artificial Sequence
  env peptide
KVVKIEPLGVAPTKA

SEQ ID NO: 249 - protein, 15 aa, Artificial Sequence
  env peptide
KVVEIKPLGVAPTKA

SEQ ID NO: 250 - protein, 15 aa, Artificial Sequence
  env peptide
KVVEIKPLGIAPTKA

SEQ ID NO: 251 - protein, 15 aa, Artificial Sequence
  env peptide
EPLGVAPTRAKRRVV

SEQ ID NO: 252 - protein, 15 aa, Artificial Sequence
  env peptide
PLGVAPTKAKRRVVQ

SEQ ID NO: 253 - protein, 15 aa, Artificial Sequence
  env peptide
IGLAPTKAKRRVVQR

SEQ ID NO: 254 - protein, 15 aa, Artificial Sequence
  env peptide
GIAPTKAKRRVVERE

SEQ ID NO: 255 - protein, 15 aa, Artificial Sequence
  env peptide
GIAPTRAKRRVVERE

SEQ ID NO: 256 - protein, 15 aa, Artificial Sequence
  env peptide
GVAPTEAKRRVVERE

SEQ ID NO: 257 - protein, 15 aa, Artificial Sequence
  env peptide
APTKARRRVVEREKR

SEQ ID NO: 258 - protein, 15 aa, Artificial Sequence
  env peptide
KAKRRVVQREKRAVG

SEQ ID NO: 259 - protein, 15 aa, Artificial Sequence
  env peptide
AKRRVVEREKRAVGI

SEQ ID NO: 260 - protein, 15 aa, Artificial Sequence
  env peptide
RVVEREKRAIGLGAM

SEQ ID NO: 261 - protein, 15 aa, Artificial Sequence
  env peptide
VQREKRAVGIGALFL

SEQ ID NO: 262 - protein, 15 aa, Artificial Sequence
  env peptide
VEREKRAVGLGALFL

SEQ ID NO: 263 - protein, 15 aa, Artificial Sequence
  env peptide
EREKRAVGIGAVFLG

SEQ ID NO: 264 - protein, 15 aa, Artificial Sequence
  env peptide
EKEKRAIGLGAMFLG

SEQ ID NO: 265 - protein, 15 aa, Artificial Sequence
  env peptide
REKRAVGLGAVFLGF

SEQ ID NO: 266 - protein, 15 aa, Artificial Sequence
  env peptide
RAVGTIGAMFLGFLG

SEQ ID NO: 267 - protein, 15 aa, Artificial Sequence
  env peptide
IGLGAMFLGFLGAAG

SEQ ID NO: 268 - protein, 15 aa, Artificial Sequence
  env peptide
VGIGALFLGFLGAAG

SEQ ID NO: 269 - protein, 15 aa, Artificial Sequence
  env peptide
VGIGAVFLGFLGVAG

SEQ ID NO: 270 - protein, 15 aa, Artificial Sequence
  env peptide
GIGAVFLGFLGAAGS

SEQ ID NO: 271 - protein, 15 aa, Artificial Sequence
  env peptide
GLGAVFLGFLGTAGS

SEQ ID NO: 272 - protein, 15 aa, Artificial Sequence
  env peptide
IGAMIFGFLGAAGST

SEQ ID NO: 273 - protein, 15 aa, Artificial Sequence
  env peptide
LGAVFIGFLGAAGST

SEQ ID NO: 274 - protein, 15 aa, Artificial Sequence
  env peptide
LGFLGAAGSTMGAAS

SEQ ID NO: 275 - protein, 15 aa, Artificial Sequence
  env peptide
AGSTMGAASITLTVQ

SEQ ID NO: 276 - protein, 15 aa, Artificial Sequence
  env peptide
AGSTMGAASMTLTVQ

SEQ ID NO: 277 - protein, 15 aa, Artificial Sequence
  env peptide
AGSTMGAASLTLTVQ

SEQ ID NO: 278 - protein, 15 aa, Artificial Sequence
  env peptide
MGARSMTLTVQARQL

SEQ ID NO: 279 - protein, 15 aa, Artificial Sequence
  env peptide
AASITLTVQARQLLS

SEQ ID NO: 280 - protein, 15 aa, Artificial Sequence
  env peptide
AASMTLTVQARLLLS

SEQ ID NO: 281 - protein, 15 aa, Artificial Sequence
  env peptide
AASLTLTVQARQLMS

SEQ ID NO: 282 - protein, 15 aa, Artificial Sequence
  env peptide
TVQARQLLSGIVQQQ

SEQ ID NO: 283 - protein, 15 aa, Artificial Sequence
  env peptide
TVQARLLLSGIVQQQ

SEQ ID NO: 284 - protein, 15 aa, Artificial Sequence
  env peptide
LLSGIVQQQSNLLRA

SEQ ID NO: 285 - protein, 15 aa, Artificial Sequence
  env peptide
LLSGIVQQQNNLLRA

SEQ ID NO: 286 - protein, 15 aa, Artificial Sequence
  env peptide
VVQQQSNLLRAIEAQ

SEQ ID NO: 287 - protein, 15 aa, Artificial Sequence
  env peptide
VQQQNNLLRAIEAQH

SEQ ID NO: 288 - protein, 15 aa, Artificial Sequence
  env peptide
VQQQSNLLKAIEAQQ

SEQ ID NO: 289 - protein, 15 aa, Artificial Sequence
  env peptide
NLLRAIEAQQHLLQL

SEQ ID NO: 290 - protein, 15 aa, Artificial Sequence
  env peptide
NLLSGIVQQQSNLLK

SEQ ID NO: 291 - protein, 15 aa, Artificial Sequence
  env peptide
LLKAIEAQQHLLKLT

SEQ ID NO: 292 - protein, 15 aa, Artificial Sequence
  env peptide
LLMAIEAQQHLLQLT

SEQ ID NO: 293 - protein, 15 aa, Artificial Sequence
  env peptide
LRAIEAQQHMLQLTA

SEQ ID NO: 294 - protein, 15 aa, Artificial Sequence
  env peptide
EAQQHMLQLTVWGIK

SEQ ID NO: 295 - protein, 15 aa, Artificial Sequence
  env peptide
AQQHLLQLTVWGIKQ

SEQ ID NO: 296 - protein, 15 aa, Artificial Sequence
  env peptide
AQQHLLKLTVWGIKQ

SEQ ID NO: 297 - protein, 15 aa, Artificial Sequence
  env peptide
AQQHLLRLTVWGIKQ

SEQ ID NO: 298 - protein, 15 aa, Artificial Sequence
  env peptide
HMLKLTVWGIKQLQA

SEQ ID NO: 299 - protein, 15 aa, Artificial Sequence
  env peptide
HMLRLTVWGIKQLQA

SEQ ID NO: 300 - protein, 15 aa, Artificial Sequence
  env peptide
VQLTVWGIKQLQTRV

SEQ ID NO: 301 - protein, 15 aa, Artificial Sequence
  env peptide
LTVWGIKQLQARVLA

SEQ ID NO: 302 - protein, 15 aa, Artificial Sequence
  env peptide
WGIKQLQARILAVER

SEQ ID NO: 303 - protein, 15 aa, Artificial Sequence
  env peptide
GIKQLQTRVLAIERY

SEQ ID NO: 304 - protein, 15 aa, Artificial Sequence
  env peptide
KQLQARVLAVERYLK

SEQ ID NO: 305 - protein, 15 aa, Artificial Sequence
  env peptide
KQLQARVLALERYLK

SEQ ID NO: 306 - protein, 15 aa, Artificial Sequence
  env peptide
KQLQARVLAIERYLQ

SEQ ID NO: 307 - protein, 15 aa, Artificial Sequence
  env peptide
TRVLAIERYLKDQQL

SEQ ID NO: 308 - protein, 15 aa, Artificial Sequence
  env peptide
TRVLAVERYLRDQQL

SEQ ID NO: 309 - protein, 15 aa, Artificial Sequence
  env peptide
VLAVERYLKDQQFLG

SEQ ID NO: 310 - protein, 15 aa, Artificial Sequence
  env peptide
VERYLKDQQLLGIWG

SEQ ID NO: 311 - protein, 15 aa, Artificial Sequence
  env peptide
VERYLRDQQLLGIWG

SEQ ID NO: 312 - protein, 15 aa, Artificial Sequence
  env peptide
VEKYLKDQQLLGLWG

SEQ ID NO: 313 - protein, 15 aa, Artificial Sequence
  env peptide
ILRNYQQWWIWGILG

SEQ ID NO: 314 - protein, 15 aa, Artificial Sequence
  env peptide
DQQLLGIWGCSGKLI

SEQ ID NO: 315 - protein, 15 aa, Artificial Sequence
  env peptide
DQQLLGLWGCSGKLI

SEQ ID NO: 316 - protein, 15 aa, Artificial Sequence
  env peptide
LGIWGCSGKHICTTN

SEQ ID NO: 317 - protein, 15 aa, Artificial Sequence
  env peptide
IWGCSGKLICTTNVP

SEQ ID NO: 318 - protein, 15 aa, Artificial Sequence
  env peptide
LWGCSGKLICTTSVP

SEQ ID NO: 319 - protein, 15 aa, Artificial Sequence
  env peptide
CSGKLICTTAVPWNS

SEQ ID NO: 320 - protein, 15 aa, Artificial Sequence
  env peptide
CSGKLICTTTVPWNS

SEQ ID NO: 321 - protein, 15 aa, Artificial Sequence
  env peptide
SGKIICTTAVPWNAS

SEQ ID NO: 322 - protein, 15 aa, Artificial Sequence
  env peptide
KLICTTNVPWNSTWS

SEQ ID NO: 323 - protein, 15 aa, Artificial Sequence
  env peptide
HICTTNVPWNASWSN

SEQ ID NO: 324 - protein, 15 aa, Artificial Sequence
  env peptide
ICTTTVPWNASWSNR

SEQ ID NO: 325 - protein, 15 aa, Artificial Sequence
  env peptide
CTTNVPWNSSWSNKS

SEQ ID NO: 326 - protein, 15 aa, Artificial Sequence
  env peptide
CTTAVPWNSSWSNRS

SEQ ID NO: 327 - protein, 15 aa, Artificial Sequence
  env peptide
CTTTVPWNSSWSNKT

SEQ ID NO: 328 - protein, 15 aa, Artificial Sequence
  env peptide
TTAVPWNASWSNKSL

SEQ ID NO: 329 - protein, 15 aa, Artificial Sequence
  env peptide
TTAVPWNTSWSNKSL

SEQ ID NO: 330 - protein, 15 aa, Artificial Sequence
  env peptide
WNSSWSNKSQEEIWN

SEQ ID NO: 331 - protein, 15 aa, Artificial Sequence
  env peptide
WNSSWSNKSLDKIWN

SEQ ID NO: 332 - protein, 15 aa, Artificial Sequence
  env peptide
WNSSWSNKTYNDIWD

SEQ ID NO: 333 - protein, 15 aa, Artificial Sequence
  env peptide
NASWSNKSLDDIWNN

SEQ ID NO: 334 - protein, 15 aa, Artificial Sequence
  env peptide
NKSQSEIWDNMTWMQ

SEQ ID NO: 335 - protein, 15 aa, Artificial Sequence
  env peptide
SQEEIWNNMTWMQWD

SEQ ID NO: 336 - protein, 15 aa, Artificial Sequence
  env peptide
LNEIWDNMTWLQWDK

SEQ ID NO: 337 - protein, 15 aa, Artificial Sequence
  env peptide
YNDIWDNMTWMQWER

SEQ ID NO: 338 - protein, 15 aa, Artificial Sequence
  env peptide
EIWNNMTWMEWEKEI

SEQ ID NO: 339 - protein, 15 aa, Artificial Sequence
  env peptide
DIWNNMTWMQWEKEI

SEQ ID NO: 340 - protein, 15 aa, Artificial Sequence
  env peptide
IWDNMTWMQWDREIS

SEQ ID NO: 341 - protein, 15 aa, Artificial Sequence
  env peptide
DNMTWMQWDKEISNY

SEQ ID NO: 342 - protein, 15 aa, Artificial Sequence
  env peptide
NNMTWMEWEREINNY

SEQ ID NO: 343 - protein, 15 aa, Artificial Sequence
  env peptide
NMTWLQWDKEISNYT

SEQ ID NO: 344 - protein, 15 aa, Artificial Sequence
  env peptide
NMTWMEWEREIDNYT

SEQ ID NO: 345 - protein, 15 aa, Artificial Sequence
  env peptide
NMTWMQWEREIDNYT

SEQ ID NO: 346 - protein, 15 aa, Artificial Sequence
  env peptide
TWMQWDREINNYTNT

SEQ ID NO: 347 - protein, 15 aa, Artificial Sequence
  env peptide
WMQWDREISNYTDTI

SEQ ID NO: 348 - protein, 15 aa, Artificial Sequence
  env peptide
WEREIDNYTSLIYTL

SEQ ID NO: 349 - protein, 15 aa, Artificial Sequence
  env peptide
REISNYTNTIYRLLE

SEQ ID NO: 350 - protein, 15 aa, Artificial Sequence
  env peptide
EISNYTDTIYRLLEV

SEQ ID NO: 351 - protein, 15 aa, Artificial Sequence
  env peptide
TGLIYTLIEESQNQQ

SEQ ID NO: 352 - protein, 15 aa, Artificial Sequence
  env peptide
TNTIYRLLEDSQNQQ

SEQ ID NO: 353 - protein, 15 aa, Artificial Sequence
  env peptide
TIYRLLEESQNQQEK

SEQ ID NO: 354 - protein, 15 aa, Artificial Sequence
  env peptide
LIEESQNQQEKNEQD

SEQ ID NO: 355 - protein, 15 aa, Artificial Sequence
  env peptide
LLEDSQNQQEKNEQE

SEQ ID NO: 356 - protein, 15 aa, Artificial Sequence
  env peptide
ESQTQQEKNEQELLA

SEQ ID NO: 357 - protein, 15 aa, Artificial Sequence
  env peptide
SQNQQEKNEKDLLAL

SEQ ID NO: 358 - protein, 15 aa, Artificial Sequence
  env peptide
NQQEKNEQDLLALDK

SEQ ID NO: 359 - protein, 15 aa, Artificial Sequence
  env peptide
NQQEKNEQELLELDK

SEQ ID NO: 360 - protein, 15 aa, Artificial Sequence
  env peptide
NQQEQNEKDLLALDK

SEQ ID NO: 361 - protein, 15 aa, Artificial Sequence
  env peptide
EKNEQELLALDKWAS

SEQ ID NO: 362 - protein, 15 aa, Artificial Sequence
  env peptide
KNEKDLLALDSWNNL

SEQ ID NO: 363 - protein, 15 aa, Artificial Sequence
  env peptide
NEKDLLALDSWKNLW

SEQ ID NO: 364 - protein, 15 aa, Artificial Sequence
  env peptide
EQDLLALDKWASLWN

SEQ ID NO: 365 - protein, 15 aa, Artificial Sequence
  env peptide
EQELLELDKWASLWS

SEQ ID NO: 366 - protein, 15 aa, Artificial Sequence
  env peptide
DLLALDKWANLWNWF

SEQ ID NO: 367 - protein, 15 aa, Artificial Sequence
  env peptide
LELDKWASLWNWFDI

SEQ ID NO: 368 - protein, 15 aa, Artificial Sequence
  env peptide
ALDSWKNLWSWFDIT

SEQ ID NO: 369 - protein, 15 aa, Artificial Sequence
  env peptide
ALDSWKNLWNWFDIT

SEQ ID NO: 370 - protein, 15 aa, Artificial Sequence
  env peptide
LDTWASLWNWFSITN

SEQ ID NO: 371 - protein, 15 aa, Artificial Sequence
  env peptide
TWASLWNWFDITNWL

SEQ ID NO: 372 - protein, 15 aa, Artificial Sequence
  env peptide
KWASLWNWFSITKWL

SEQ ID NO: 373 - protein, 15 aa, Artificial Sequence
  env peptide
SWKNLWNWFDISNWL

SEQ ID NO: 374 - protein, 15 aa, Artificial Sequence
  env peptide
WANLWNWFDITKWLW

SEQ ID NO: 375 - protein, 15 aa, Artificial Sequence
  env peptide
LWNWFSITNWLWYIK